(19) United States
(12) Patent Application Publication
(10) Pub. No.: US 2006/0216709 A1
(43) Pub. Date: Sep. 28, 2006

(54) Midkine-like protein

(76) Inventors: Carmit Levita, London (GB); Richard Joseph Fagan, London (GB); David Michalovich, London (GB); Melanie Yorke, Confignon (CH)

Correspondence Address: SALIWANCHIK LLOYD & SALIWANCHIK;A PROFESSIONAL ASSOCIATION, PO BOX 142950, GAINESVILLE, FL, 32614-2950, US

(21) Appl. No.: 10/538,195

(22) PCT Filed: Dec. 10, 2003
(86) PCT No.: PCT/GB03/05374

§ 371 Date: Feb. 27, 2006

(51) Int. Cl. C12Q 1/68; C07H 21/04; C12P 21/02; C07K 14/52; C07K 16/24

(52) U.S. Cl. 435/6; 435/69.5; 435/320.1; 435/325; 530/351; 530/388.23; 536/23.5

(57) ABSTRACT

The invention is based on the discovery that the INSP106 protein is a novel splice variant of a known midkine family member (swall|P21741|MK_HUMAN).

This invention relates to a novel protein, termed INSP106, herein identified as a novel splice variant of a known midkine family member (swall|P21741|MK_HUMAN) and to the use of this protein and nucleic acid sequences from the encoding genes in the diagnosis, prevention and treatment of disease. A gene model illustrating the variation within INSP106 in comparison to the known midkine shows that the 3^{rd} coding exon has been extended in the 3′ direction instead of having a separate 4^{th} coding exon present in P21741 (FIG. 1).

All publications, patents and patent applications cited herein are incorporated in full by reference.

### BACKGROUND

The process of drug discovery is presently undergoing a fundamental revolution as the era of functional genomics comes of age. The term “functional genomics” applies to an approach utilising bioinformatics tools to ascribe function to protein sequences of interest. Such tools are becoming increasingly necessary as the speed of generation of sequence data is rapidly outpacing the ability of research laboratories to assign functions to these protein sequences.

As bioinformatics tools increase in potency and in accuracy, these tools are rapidly replacing the conventional techniques of biochemical characterisation. Indeed, the advanced bioinformatics tools used in identifying the present invention are now capable of outputting results in which a high degree of confidence can be placed.

Various institutions and commercial organisations are examining sequence data as they become available and significant discoveries are being made on an on-going basis. However, there remains a continuing need to identify and characterise further genes and the polypeptides that they encode, as targets for research and for drug discovery.

Recently, a remarkable tool for the evaluation of sequences of unknown function has been developed by the Applicant for the present invention. This tool is a database system, termed the Biopendium search database, that is the subject of WO 01/69507. This database system consists of an integrated data resource created using proprietary technology and containing information generated from an all-by-all comparison of all available protein or nucleic acid sequences.

The aim behind the integration of these sequence data from separate data resources is to combine as much data as possible, relating both to the sequences themselves and to information relevant to each sequence, into one integrated resource. AR the available data relating to each sequence, including data on the three-dimensional structure of the encoded protein, if this is available, are integrated together to make best use of the information that is known about each sequence and thus to allow the most educated predictions to be made from comparisons of these sequences. The annotation that is generated in the database and which accompanies each sequence entry imparts a biologically relevant context to the sequence information.

This data resource has made possible the accurate prediction of protein function from sequence alone. Using conventional technology, this is only possible for proteins that exhibit a high degree of sequence identity (above about 20%-30% identity) to other proteins in the same functional family. Accurate predictions are not possible for proteins that exhibit a very low degree of sequence homology to other related proteins of known function.

Alternative pre-mRNA splicing is a major cellular process by which functionally diverse proteins can be generated from the primary transcript of a single gene, often in tissue specific patterns.

Experimentally, splice variants are identified by the fortuitous isolation and subsequent sequencing of variant mRNAs. However, this experimental approach has not been exhaustively completed for the human transcriptome (since this would require systematic isolation and sequencing of all mRNAs from all human tissues under all possible environmental conditions) and due to this experimental limitation there remains a large number of splice variants which have yet to be identified.

We have used proprietary bioinformatic approaches to perform a purposeful, directed search for the existence of splice variants of the human midkine gene. By this method the limited data set of experimentally known splice variants can be extended to a much larger set of predicted splice variants.

Introduction to Secreted Proteins

The ability of cells to make and secrete extracellular proteins is central to many biological processes. Enzymes, growth factors, extracellular matrix proteins and signalling molecules are all secreted by cells. This is through fusion of a secretory vesicle with the plasma membrane. In most cases, but not all, proteins are directed to the endoplasmic reticulum and into secretory vesicles by a signal peptide. Signal peptides are cis-acting sequences that affect the transport of polypeptide chains from the cytoplasm to a membrane bound compartment such as a secretory vesicle. Polypeptides that are targeted to the secretory vesicles are either secreted into the extracellular matrix or are retained in the plasma membrane. The polypeptides that are retained in the plasma membrane will have one or more transmembrane domains. Examples of secreted proteins that play a central role in the functioning of a cell are cytokines, hormones, extracellular matrix proteins (adhesion molecules), proteases and other growth and differentiation factors.

Growth Factors

Growth factors represent a relatively large group of polypeptides which share the common property of inducing cell multiplication both in vivo and in vitro. Growth factors differ from classical endocrine hormones such as insulin or growth hormone in two important ways. Firstly, endocrine hormones are typically synthesised in specialised glands (such as the pancreas, in the case of insulin) whereas growth factors are often synthesised in multiple types of cells and tissues. Secondly, classical endocrine hormones are released into body fluids at the site of synthesis and are carried to their target tissue in the bloodstream. A hallmark of growth factors is that, in most instances, they act locally within the tissues in which they are synthesised (reviewed in Heath, J K. (1993) Growth Factors, Oxford University Press, Oxford, UK, pp. 15-33).

Although the level of sequence similarity is not high, growth factors can be classified into families. There is the platelet-derived growth factor family (PDGF-A, PDGF-B and VEGF), the epidermal growth factor family (EGF, TGF-alpha, amphiregulin), the fibroblast growth factor family (FGF, FGF4, FGF5, and FGF7), the insulin like growth factors (IGF-I, IGH-II, and proinsulin), the neurotrophic growth factor family (NGF) and the transforming growth factor family TGF-beta 1-3, bone morphogenetic proteins 2-7, and mullerian inhibitory substance).

Growth factors are extracellular and in order to exert a biological effect they interact with specific, high affinity receptors located on the plasma membranes of target cells. The molecular characterisation of a variety of different growth factor receptors revealed that they fall into defined families; the tyrosine kinase receptors, G-protein associated seven helical receptors, and the serine/threonine kinase receptors. The tyrosine kinase receptors are characterised by an extracellular domain, a transmembrane domain, and an intracellular domain which possess tyrosine kinase activity. The serine/threonine kinase growth factor receptors are similar to the tyrosine kinase receptors with an extracellular domain, a tramembrane domain, and an intracellular domain. The intracellular domain has intrinsic serine/threonine kinase activity.

Dys-regulation of growth factor function results in many different disease phenotypes, including, but not exclusive to oncology (Bartucci M et al, (2001) Cancer Res. September 15;61(18):6747-54, Dias S et al., (2001) Proc. Natl. Acad. Sci. U. S. A. September 11;98(19):10857-62, Djavan B et al., (2001) World J Urol. August;19(4):225-33), inflammation (Fiocchi C. (2001) J Clin Invest. August;108(4):523-6, Hodge S et al.,(2001) Respirology. September;6(3):205-211, Fenwick S A et al., (2001) J Anat. September;199(Pt 3):231-40), neurological disorders (Cooper J D et al., (2001) Proc Natl Acad Sci U S A. August 28;98(18):10439-44, Fahnestock M et al, (2001) Mol Cell Neurosci. August;18(2):210-20), metabolic disorders (Vickers M H et al., (2001) Endocrinology. September;142(9):3964-73), atherosclerosis (Oemar, B. S., et al. (1997) Human connective tissue growth factor is expressed in advanced atherosclerotic lesions. Circulation, 95(4), 831-839) and fibrotic disorders such as scleroderma (Isarahi, A. et al. (1995) Significant correlation between connective tissue growth factor gene expression and skin sclerosis in tissue sections from patients with systemic sclerosis. J. Invest. Dermatol. 105, 280-284) and diabetic nephropathy (Abdel Wahab, N. et al. (1996) Expression of extracellular matrix molecules in human mesangial cells in response to prolonged hyperglycemia. Biochem. J. 316, 985-992).

Midkine (MK) is a 13 kDa heparin-binding polypeptide which enhances neurite outgrowth, neuronal cell survival and plasminogen activator activity. MK is structurally divided into two domains, and most of the biological activities are located on the C-terminal domain. The MK dimer has been shown to be the active form, giving signals to endothelial cells and probably to neuronal cells. A head-to-head dimer model of MK has been postulated (Iwasaki et al, 1997). The dimer has a fused heparin-binding site at the dimer interface of the C-terminal domain, and the heparin-binding sites on MK fit the sulfate group clusters on heparin. These features are consistent with the proposed stronger heparin-binding activity and biological activity of the dimer.

Reported midkine functions include maintaining and differentiating embryonic nerve cells and enhancing neurite extension; promoting division of specific cell lines (Muramatsu, H. et al., Biochem. Biophys. Res. Commun. 177: 652-658, 1991; and Michikawa, M. et al., J. Neurosci. Res. 35: 530-539, 1993; Muramatsu, H. et al., Dev. Biol. 159: 392402, 1993); regulating embryonic development (Kadomatsu, K. et al., J. Cell. Biol. 110: 607-616, 1990; Mitsiadis, T. A. et al., Development 121: 37-51, 1995); etc. Furthermore, anti-midkine antibody reportedly inhibits dentition in vitro (Mitsiadis, T. A. et al, J. Cell. Biol. 129: 267-281, 1995). MK is also known to promote plasminogen activator activity in bovine aortic endothelial cells, leading to increased fibrinolysis (Kojima et al., 1995).

It has been revealed that midkine plays crucial roles in restoration of damaged tissues and some diseases. The expression patterns of midkine were investigated in various human carcinomas. The studies revealed that midkine expression is elevated in various cancers including stomach cancer, colon cancer, pancreatic cancer, lung cancer, thoracic cancer, and liver cancer (Tsutsui, J. et al., Cancer Res. 53: 1281-1285, 1993; Aridome, K. et al., Jap. J. Cancer Res. 86: 655-661, 1995; and Garver, R. I. et al., Cancer 74: 1584-1590, 1994). The high-level expression of midkine correlates with unfavorable prognoses in patients affected with neuroblastoma (Nakagawara, A. et al., Cancer Res. 55: 1792-1797, 1995). Midkine accumulates in senile plaques of most patients with Alzheimer's disease (Yasuhara, O. et al. Biochem. Biophys. Res. Commun. 192: 246-251, 1993). Midkine is expressed in regions with edema at early stages of cerebral infarction (Yoshida, Y. et al., Dev. Brain Res. 85: 25-30, 1995). These findings indicate that midkine may be associated with restoration of damaged tissues and tissue abnormalities that are signs of some diseases.

Midkine activity has also been linked to neutrophilic functional disorders (e.g. lazy-leukocyte (chemotaxis-deficient leukocyte) syndrome) and inflammatory diseases (EP 998,941).

Identification of secreted proteins and, in particular, growth factors, is therefore of extreme importance in increasing the understanding of the underlying pathways that lead to the disease states and associated disease states, mentioned above, and in developing more effective gene and/or drug therapies to treat these disorders.

### THE INVENTION

The invention is based on the discovery that the INSP106 protein is a novel splice variant of a known midkine family member (swall|P21741|MK_HUMAN).

A gene model illustrating the variation within INSP106 in comparison to the known midkine shows that the 3^{rd} coding exon has been extended in the 3′ direction instead of having a separate 4^{th} coding exon present in P21741 (FIG. 1).

The multiple alignment demonstrates how a hydrophobic proline ‘tract’ extends the INSP106 prediction in the C terminal tail (FIG. 2).

It has been indicated that the removal of the C terminal tail reduces the neurite promoting activity as well as the plasminogen activator enhancing activity of the midkines. The tails have also been found to ‘facilitate the steric arrangement of the N and C domain so that the function of the C-domain in binding heparin is not disturbed’ (Akhter et al, 1998). Recently published data has shown that the removal of the lysine rich C-terminal tail of a pleiotrophin that shares 50% homology with midkine prevents binding to the receptor (Pierrot et al, 2001).

The binding of midkine to heparin requires the formation of a dimer in a head to head fashion involving both the N and C domains (Iwasaki et al, 1997). The midkine dimer binds to a protein tyrosine phosphatase E (PTPE) receptor to initiate the MAP kinase pathway (Muramatsu et al, 2002). Without wishing to be bound to any particular theory, we believe that INSP106 may act as a dominant negative antagonist; the proline ‘tract’ may interfere with receptor binding (FIG. 3).

In one embodiment of the first aspect of the invention, there is provided a polypeptide which does not comprise the amino acid sequence as recited in SEQ ID NO:10 (exon 4 of the known midkine family member (swall|P21741|MK_HUMAN)) but which:
(i) comprises or consists of the amino acid sequence as recited in SEQ ID NO:2 (exon 3 of INSP016);
(ii) comprises or consists of a fragment of SEQ ID NO:2 which fragment comprises at least a fragment of SEQ ID NO:8 (preferably, at least 1, 2, 3, 4, 6, 8, 10, 12 or 14 amino acids) and wherein the polypeptide has the activity of SEQ ID NO:4 or SEQ ID NO:6 (the full length INSP0106 sequence with or without the signal peptide sequence respectively) or has an antigenic determinant which is specific to a polypeptide comprising SEQ ID NO:2; or
(iii) comprises or consists of a functional equivalent of (i) or (ii) which has the activity of SEQ ID NO:4 or SEQ ID NO:6 or has an antigenic determinant which is specific to a polypeptide comprising SEQ ID NO:2.

The polypeptide having the sequence recited in SEQ ID NO:2 is referred to hereafter as “the INSP106 exon 3nov polypeptide”. The polypeptide having the sequence recited in SEQ ID NO:4 is referred to hereafter as “the INSP106 full length polypeptide including signal peptide”. The polypeptide having the sequence recited in SEQ ID NO:6 is referred to hereafter as “the INSP106 full length polypeptide excluding signal peptide”. The polypeptide having the sequence recited in SEQ ID NO:8 is referred to hereafter as “the INSP106 extended portion of exon 3 polypeptide”. This sequence thus comprises the amino acids that form the extended portion of exon 3 of the known midkine P21741.

The polypeptide having the sequence recited in SEQ ID NO:10 is exon 4 of swall|P21741|MK_HUMAN and does not form part of the present invention.

The term “INSP106 polypeptides” as used herein includes polypeptides comprising or consisting of the INSP106 exon 3nov polypeptide, the INSP106 full length polypeptide including signal peptide, the INSP 106 full length polypeptide excluding signal peptide, and the INSP106 extended portion of exon 3 polypeptide.

By a polypeptide which has “the activity of SEQ ID NO:4 or SEQ ID NO:6” we refer to a polypeptide present in a monomeric or polymeric (e.g. dimeric) form).

Preferably, a polypeptide which has “the activity of SEQ ID NO:4 or SEQ ID NO:6” has reduced neurite promoting activity and/or reduced plasminogen activator enhancing activity of the midkines as compared with swall|P21741|MK_HUMAN.

Preferably, a polypeptide which has “the activity of SEQ ID NO:4 or SEQ ID NO:6” modulates (and preferably antagonises) the activity of swall|P21741|MK_HUMAN (SEQ ID NO.12). Preferably, a polypeptide which has “the activity of SEQ ID NO:4 or SEQ ID NO:6” inhibits the neurite promoting activity of swall|P21741|MK_HUMAN (SEQ ID NO.12) or inhibits the plasminogen activator enhancing activity of swall|P21741|MK_HUMAN (SEQ ID NO.12).

Whilst not wishing to be bound by any particular theory, it is believed that the protein of the present invention may modulate (e.g. antagonise) swall|P21741|MK_HUMAN and that this ability may be due to the extended C-terminal tail of INSP0106.

By a polypeptide which has “an antigenic determinant which is specific to a polypeptide comprising SEQ ID NO:2” we refer to antigenic determinants which are possessed by the polypeptide of SEQ ID NO:4 or SEQ ID NO:6 but which are not possessed by the known midkine family member swall|P21741|MK_HUMAN. Such antigenic determinants may consist of an amino acid sequence located within SEQ ID NO:8 or may consist of an amino acid sequence which spans the junction of SEQ ID NO:8 with the portion of SEQ ID NO:2 which is located at the N-terminal side of SEQ ID NO:8 (i.e. the amino acid sequence which corresponds to the sequence encoded by exon 3 of P21741). As discussed below, such antigenic determinants can be used to generate ligands, such as polyclonal or monoclonal antibodies, that are immunospecific for the polypeptides of the invention.

In a preferred aspect of (i) of the first aspect of the invention there is provided a polypeptide which comprises (and preferably consists) of the amino acid sequence as recited in SEQ ID NO:4 or in SEQ ID NO:6, or a functional equivalent thereof.

Preferably, the polypeptides of the first aspect of the invention comprise the amino acid sequence as recited in SEQ ID NO:2 or in SEQ ID NO:8 or at least 70%, 80%, 90% or 95% of the amino acid sequence as recited in SEQ ID NO:2 or in SEQ ID NO:8. In another embodiment of the first aspect of the invention, it is preferred that functional equivalents of the polypeptides of the invention comprise an amino acid sequence having at least 70%, 80%, 90% or 95% sequence identity to the amino acid sequence as recited in SEQ ID NO:2 or in SEQ ID NO:8 or to a sequence representing at least 70%, 80%, 90% or 95% of the amino acid sequence as recited in SEQ ID NO:2 or in SEQ ID NO:8.

Preferably, the polypeptides of the first aspect of the invention do not comprise: a fragment of the amino acid sequence as recited in SEQ ID NO:10; an amino acid sequence having at least 40%, 50%, 60%, 70%, 80%, 90% or 95% sequence identity with the amino acid sequence as recited in SEQ ID NO:10; or an amino acid sequence having at least 60%, 70%, 80%, 90% or 95% sequence identity with a fragment representing at least 70%, 80%, 90% or 95% of the amino acid sequence as recited in SEQ ID NO:10.

In a second aspect, the invention provides a purified nucleic acid molecule which encodes a polypeptide of the first aspect of the invention.

In a first embodiment of this aspect of the invention, the purified nucleic acid molecule comprises or consists of the nucleic acid sequence as recited in SEQ ID NO:1 (encoding the INSP106 exon 3nov-polypeptide), SEQ ID NO:3 (encoding the INSP106 full length polypeptide including signal peptide), SEQ ID NO:5 (encoding the INSP106 full length polypeptide excluding signal peptide), SEQ ID NO:7 (encoding the INSP106 extended portion of exon 3 polypeptide), or is a redundant equivalent or fragment of any one of these sequences.

The coding sequence (and polypeptide encoded thereby) for the known midkine family member (swall|P21741|MK_HUMAN) is specifically excluded from the scope of the present invention. Similarly, the ESTs set forth in Table 1 are specifically excluded from the scope of the present invention.

TABLE 1
ESTs identified covering gene model of splice variant INSP106

| EST accession | | | |
|---|---|---|---|
| numbers | Image ID | Tissue distribution | Read direction |
| BI820606.1 | 5176115 | Brain/Lung/Testis | 5′ |
| BI914771.1 | 5248117 | Brain | 5′ |
| AL553751.1 | — | Placenta | 5′ |
| AL516918 | — | Brain | 5′ |

In one preferred embodiment of the second aspect of the invention, there is provided a nucleic acid comprising or consisting of the nucleotide sequence set forth in FIG. 6. The nucleic acid molecule may be single-stranded (comprising or consisting of the nucleotide sequence set forth in FIG. 6, or the complement thereof) or double-stranded.

In a third aspect, the invention provides a purified nucleic acid molecule which hybridizes under high stringency conditions with SEQ ID NO.3 or SEQ ID NO.5 but which does not hybridise under high stringency conditions to P21741 (SEQ ID NO.11). Preferably, there is provided a nucleic acid molecule according to the third aspect of the invention which hybridizes under high stringency conditions with SEQ ID NO.2. In an alternative embodiment of the third aspect of the invention there is provided a nucleic acid molecule which hybridises under high stringency conditions with a nucleic acid sequence which spans the junction between the portion of the third exon which is common to P21741 and the INSP0106 (i.e. the sequence equivalent to exon 3 of P21741) and the extended third exon sequence set forth in SEQ ID NO.7 (encoding the INSP106 extended portion of exon 3 polypeptide).

In a fourth aspect, the invention provides a vector, such as an expression vector, that contains a nucleic acid molecule of the second or third aspect of the invention.

In a fifth aspect, the invention provides a host cell transformed with a vector of the fourth aspect of the invention.

In a sixth aspect, the invention provides a ligand which binds specifically to a polypeptide of the first aspect of the invention.

Ligands to a polypeptide according to the invention may come in various forms, including natural or modified substrates, enzymes, receptors, small organic molecules such as small natural or synthetic organic molecules of up to 2000Da, preferably 800Da or less, peptidomimetics, inorganic molecules, peptides, polypeptides, antibodies, structural or functional mimetics of the aforementioned.

Such compounds may be identified using the assays and screening methods disclosed herein.

In a seventh aspect, the invention provides a compound that is effective to alter the expression of a natural gene which encodes a polypeptide of the first aspect of the invention or to regulate the activity of a polypeptide of the first aspect of the invention.

A compound of the seventh aspect of the invention may either increase (agonise) or decrease (antagonise) the level of expression of the gene or the activity of the polypeptide.

Importantly, the identification of the function of the INSP106 polypeptide allows for the design of screening methods capable of identifying compounds that are effective in the treatment and/or diagnosis of disease. Ligands and compounds according to the sixth and seventh aspects of the invention may be identified using such methods. These methods are included as aspects of the present invention.

In an eighth aspect, the invention provides a polypeptide of the first aspect of the invention, or a nucleic acid molecule of the second or third aspect of the invention, or a vector of the fourth aspect of the invention, or a ligand of the sixth aspect of the invention, or a compound of the seventh aspect of the invention, for use in therapy or diagnosis of a disease in which midkines are implicated. Such diseases and disorders may include reproductive disorders, cell proliferative disorders, including neoplasm, melanoma, lung, colorectal, breast, pancreas, head and neck and other solid tumours; stomach cancer, colon cancer, pancreatic cancer, lung cancer, thoracic cancer, and liver cancer; myeloproliferative disorders, such as leukemia, non-Hodgkin lymphoma, leukopenia, thrombocytopenia, angiogenesis disorder, Kaposis' sarcoma; autoimmune/inflammatory disorders, including allergy, inflammatory bowel disease, pancreatitis, arthritis, psoriasis, psoriasis vulgaris, respiratory tract inflammation, asthma, and organ transplant rejection; cardiovascular disorders, including hypertension, oedema, angina, atherosclerosis, thrombosis, sepsis, shock, reperfusion injury, and ischemia, particularly ischemic heart disease; neurological disorders including central nervous system disease, Alzheimer's disease, brain injury, Parkinson's disease, amyotrophic lateral sclerosis, and pain; developmental disorders; metabolic disorders including diabetes mellitus, osteoporosis, and obesity, AIDS, renal disease, particularly idiopathic nephrotic syndrome; disorders related to fibrinolysis; neutrophilic functional disorders (e.g. lazy-leukocyte (chemotaxis-deficient leukocyte) syndrome); inflammatory diseases; wound healing disorders; lung injury; infections including viral infection, bacterial infection, fungal infection and parasitic infection and other pathological conditions.

Preferably, the moieties of the invention (a polypeptide of the first aspect of the invention, or a nucleic acid molecule of the second or third aspect of the invention, or a vector of the fourth aspect of the invention, or a ligand of the sixth aspect of the invention, or a compound of the seventh aspect of the invention) may be used in the treatment of disorders in which aberrant neurite growth promoting activity, neurite function, plasminogen activating activity, heparin binding activity, survival/differentiation of ES cell lines, regulation of embryonic development and dentition are implicated.

These moieties may also be used in the manufacture of a medicament for the treatment of the above-mentioned diseases and disorders.

In a ninth aspect, the invention provides a method of diagnosing a disease in a patient, comprising assessing the level of expression of a natural gene encoding a polypeptide of the first aspect of the invention or the activity of a polypeptide of the first aspect of the invention in tissue from said patient and comparing said level of expression or activity to a control level wherein a level that is different to said control level is indicative of disease. Such a method will preferably be carried out in vitro. Similar methods may be used for monitoring the therapeutic treatment of disease in a patient, wherein altering the level of expression or activity of a polypeptide or nucleic acid molecule over the period of time towards a control level is indicative of regression of disease.

A preferred method for detecting polypeptides of the first aspect of the invention comprises the steps of. (a) contacting a ligand, such as an antibody, of the sixth aspect of the invention with a biological sample under conditions suitable for the formation of a ligand-polypeptide complex; and (b) detecting said complex.

A number of different methods according to the ninth aspect of the invention exist, as the skilled reader will be aware, such as methods of nucleic acid hybridisation with short probes, point mutation analysis, polymerase chain reaction (PCR) amplification and methods using antibodies to detect aberrant protein levels. Similar methods may be used on a short or long term basis to allow therapeutic treatment of a disease to be monitored in a patient. The invention also provides kits that are useful in these methods for diagnosing disease.

Preferably, the disease diagnosed by a method of the ninth aspect of the invention is a disease in which midkines are implicated.

In a tenth aspect, the invention provides for the use of the polypeptides of the first aspect of the invention as a growth factor or as a modulator of growth factor activity. Preferably, the invention provides for the use of the polypeptides of the first aspect of the invention as a modulator of P21741-like activity; preferably as an antagonist of P21741-like activity. By “P21741-like activity” we refer to activity possessed by P21741 (but which may also be possessed by similar proteins), e.g. heparin-binding activity, neurite outgrowth enhancing activity, promotion of neuronal survival and plasminogen activator activity. Suitable uses of the polypeptides of the invention include use as a regulator of cellular growth, metabolism or differentiation, use as part of a receptor/ligand pair and use as a diagnostic marker for a physiological or pathological condition, such as one of those listed above.

The polypeptides of the invention may also be used for the modulation of (e.g. the antagonisation of) neurite growth, neuronal cell survival, plasminogen activation, heparin-binding, the maintenance and differentiation of embryonic nerve cells, the maintenance and differentiation of ES cell lines, to regulate embryonic development, dentition and tissue repair.

In an eleventh aspect, the invention provides a pharmaceutical composition comprising a polypeptide of the first aspect of the invention, or a nucleic acid molecule of the second or third aspect of the invention, or a vector of the fourth aspect of the invention, or a ligand of the sixth aspect of the invention, or a compound of the seventh aspect of the invention, in conjunction with a pharmaceutically-acceptable carrier.

In a twelfth aspect, the present invention provides a polypeptide of the first aspect of the invention, or a nucleic acid molecule of the second or third aspect of the invention, or a vector of the fourth aspect of the invention, or a ligand of the sixth aspect of the invention, or a compound of the seventh aspect of the invention, for use in therapy or diagnosis. These molecules may also be used in the manufacture of a medicament for the treatment of a disease.

In a thirteenth aspect, the invention provides a method of treating a disease in a patient comprising administering to the patient a polypeptide of the first aspect of the invention, or a nucleic acid molecule of the second or third aspect of the invention, or a vector of the fourth aspect of the invention, or a ligand of the sixth aspect of the invention, or a compound of the seventh aspect of the invention.

For diseases in which the expression of a natural gene encoding a polypeptide of the first aspect of the invention, or in which the activity of a polypeptide of the first aspect of the invention, is lower in a diseased patient when compared to the level of expression or activity in a healthy patient, the polypeptide, nucleic acid molecule, ligand or compound administered to the patient should be an agonist. Conversely, for diseases in which the expression of the natural gene or activity of the polypeptide is higher in a diseased patient when compared to the level of expression or activity in a healthy patient, the polypeptide, nucleic acid molecule, ligand or compound administered to the patient should be an antagonist. Examples of such antagonists include antisense nucleic acid molecules, ribozymes and ligands, such as antibodies.

Preferably, the disease is a disease in which midkines are implicated.

In a fourteenth aspect, the invention provides transgenic or knockout non-human animals that have been transformed to express higher, lower or absent levels of a polypeptide of the first aspect of the invention. Such transgenic animals are very useful models for the study of disease and may also be used in screening regimes for the identification of compounds that are effective in the treatment or diagnosis of such a disease.

A summary of standard techniques and procedures which may be employed in order to utilise the invention is given below. It will be understood that this invention is not limited to the particular methodology, protocols, cell lines, vectors and reagents described. It is also to be understood that the terminology used herein is for the purpose of describing particular embodiments only and it is not intended that this terminology should limit the scope of the present invention. The extent of the invention is limited only by the terms of the appended claims.

Standard abbreviations for nucleotides and amino acids are used in this specification.

The practice of the present invention will employ, unless otherwise indicated, conventional techniques of molecular biology, microbiology, recombinant DNA technology and immunology, which are within the skill of those working in the art.

Such techniques are explained fully in the literature. Examples of particularly suitable texts for consultation include the following: Sambrook Molecular Cloning; A Laboratory Manual, Second Edition (1989); DNA Cloning, Volumes I and II (D. N Glover ed. 1985); Oligonucleotide Synthesis (M. J. Gait ed. 1984); Nucleic Acid Hybridization (B. D. Hames & S. J. Higgins eds. 1984); Transcription and Translation (B. D. Hames & S. J. Higgins eds. 1984); Animal Cell Culture (R. I. Freshney ed. 1986); Immobilized Cells and Enzymes (IRL Press, 1986); B. Perbal, A Practical Guide to Molecular Cloning (1984); the Methods in Enzymology series (Academic Press, Inc.), especially volumes 154 & 155; Gene Transfer Vectors for Mammalian Cells (J. H. Miller and M. P. Calos eds. 1987, Cold Spring Harbor Laboratory); Immunochemical Methods in Cell and Molecular Biology (Mayer and Walker, eds. 1987, Academic Press, London); Scopes, (1987) Protein Purification: Principles and Practice, Second Edition (Springer Verlag, N.Y.); and Handbook of Experimental Immunology, Volumes I-IV (D. M. Weir and C. C. Blackwell eds. 1986).

As used herein, the term “polypeptide” includes any peptide or protein comprising two or more amino acids joined to each other by peptide bonds or modified peptide bonds, i.e. peptide isosteres. This term refers both to short chains (peptides and oligopeptides) and to longer chains (proteins).

The polypeptide of the present invention may be in the form of a mature protein or may be a pre-, pro- or prepro-protein that can be activated by cleavage of the pre-, pro- or prepro-portion to produce an active mature polypeptide. In such polypeptides, the pre-, pro- or prepro-sequence may be a leader or secretory sequence or may be a sequence that is employed for purification of the mature polypeptide sequence.

The polypeptide of the first aspect of the invention may form part of a fusion protein. For example, it is often advantageous to include one or more additional amino acid sequences which may contain secretory or leader sequences, pro-sequences, sequences which aid in purification, or sequences that confer higher protein stability, for example during recombinant production. Alternatively or additionally, the mature polypeptide may be fused with another compound, such as a compound to increase the half-life of the polypeptide (for example, polyethylene glycol).

Polypeptides may contain amino acids other than the 20 gene-encoded amino acids, modified either by natural processes, such as by post-translational processing or by chemical modification techniques which are well known in the art. Among the known modifications which may commonly be present in polypeptides of the present invention are glycosylation, lipid attachment, sulphation, gamma-carboxylation, for instance of glutamic acid residues, hydroxylation and ADP-ribosylation. Other potential modifications include acetylation, acylation, amidation, covalent attachment of flavin, covalent attachment of a haeme moiety, covalent attachment of a nucleotide or nucleotide derivative, covalent attachment of a lipid derivative, covalent attachment of phosphatidylinositol, cross-linking, cyclization, disulphide bond formation, demethylation, formation of covalent cross-links, formation of cysteine, formation of pyroglutamate, formylation, GPI anchor formation, iodination, methylation, myristoylation, oxidation, proteolytic processing, phosphorylation, prenylation, racemization, selenoylation, transfer-RNA mediated addition of amino acids to proteins such as arginylation, and ubiquitination.

Modifications can occur anywhere in a polypeptide, including the peptide backbone, the amino acid side-chains and the amino or carboxyl termini. In fact, blockage of the amino or carboxyl terminus in a polypeptide, or both, by a covalent modification is common in naturally-occurring and synthetic polypeptides and such modifications may be present in polypeptides of the present invention.

The modifications that occur in a polypeptide often will be a function of how the polypeptide is made. For polypeptides that are made recombinantly, the nature and extent of the modifications in large part will be determined by the post-translational modification capacity of the particular host cell and the modification signals that are present in the amino acid sequence of the polypeptide in question. For instance, glycosylation patterns vary between different types of host cell.

The polypeptides of the present invention can be prepared in any suitable manner. Such polypeptides include isolated naturally-occurring polypeptides (for example purified from cell culture), recombinantly-produced polypeptides (including fusion proteins), synthetically-produced polypeptides or polypeptides that are produced by a combination of these methods.

The functionally-equivalent polypeptides of the first aspect of the invention may be polypeptides that are homologous to the INSP106 polypeptides. Two polypeptides are said to be “homologous”, as the term is used herein, if the sequence of one of the polypeptides has a high enough degree of identity or similarity to the sequence of the other polypeptide. “Identity” indicates that at any particular position in the aligned sequences, the amino acid residue is identical between the sequences. “Similarity” indicates that, at any particular position in the aligned sequences, the amino acid residue is of a similar type between the sequences. Degrees of identity and similarity can be readily calculated (Computational Molecular Biology, Lesk, A. M., ed., Oxford University Press, New York, 1988; Biocomputing. Informatics and Genome Projects, Smith, D. W., ed., Academic Press, New York, 1993; Computer Analysis of Sequence Data, Part 1, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991).

Homologous polypeptides therefore include natural biological variants (for example, allelic variants or geographical variations within the species from which the polypeptides are derived) and mutants (such as mutants containing amino acid substitutions, insertions or deletions) of the INSP106 polypeptides. Such mutants may include polypeptides in which one or more of the amino acid residues are substituted with a conserved or non-conserved amino acid residue (preferably a conserved amino acid residue) and such substituted amino acid residue may or may not be one encoded by the genetic code. Typical such substitutions are among Ala, Val, Leu and Ile; among Ser and Thr; among the acidic residues Asp and Glu; among Asn and Gln; among the basic residues Lys and Arg; or among the aromatic residues Phe and Tyr. Particularly preferred are variants in which several, i.e. between 5 and 10, 1 and 5, 1 and 3, 1 and 2 or just 1 amino acids are substituted, deleted or added in any combination. Especially preferred are silent substitutions, additions and deletions, which do not alter the properties and activities of the protein. Also especially preferred in this regard are conservative substitutions. Such mutants also include polypeptides in which one or more of the amino acid residues includes a substituent group.

Preferably, functionally equivalent polypeptides of the first aspect of the invention have a degree of sequence identity with the INSP106 polypeptides, or with active fragments thereof, of greater than 70%, 80% or 90% over the fulll length of the INSP106 sequence. More preferred polypeptides have degrees of identity of greater than 92%, 95%, 98% or 99% over the full length of the INSP106 sequence, respectively.

Preferably, the functionally equivalent polypeptides of the first aspect of the invention comprise a sequence having a degree of sequence identity with the amino acid sequence recited in SEQ ID NO. 8 of greater than 60%, 70%, 80% 90%, 92%, 95%, 98% or 99%.

In one embodiment there is provided a functionally equivalent polypeptide of the first aspect of the invention which comprises at least a fragment of SEQ ID NO: 8.

Preferably, the functionally equivalent polypeptides of the first aspect of the invention comprise SEQ ID NO: 2 or SEQ ID NO: 8.

The functionally-equivalent polypeptides of the first aspect of the invention may also be polypeptides which have been identified using one or more techniques of structural alignment. For example, the Inpharmatica Genome Threader™ technology that forms one aspect of the search tools used to generate the Biopendium search database may be used (see WO 01/69507) to identify polypeptides of presently-unknown function which, while having low sequence identity as compared to the INSP106 polypeptide, are predicted to have the activity of SEQ ID NO:4 or SEQ ID NO:6, said method utilising a polypeptide of the first aspect of the invention, by virtue of sharing significant structural homology with the INSP106 polypeptide sequences. By “significant structural homology” is meant that the Inpharmatica Genome Threader™ predicts two proteins to share structural homology with a certainty of at least 10% and above.

The polypeptides of the first aspect of the invention also include fragments of the INSP106 polypeptides and fragments of the functional equivalents of the INSP106 polypeptides, provided that those fragments retain the activity of SEQ ID NO:4 or SEQ ID NO:6 or have an antigenic determinant which is specific to a polypeptide comprising SEQ ID NO:2.

As used herein, the term “fragment” refers to a polypeptide having an amino acid sequence that is the same as part, but not all, of the amino acid sequence of the INSP106 polypeptides or one of its functional equivalents. The fragments should comprise at least n consecutive amino acids from the sequence and, depending on the particular sequence, n preferably is 7 or more (for example, 8, 10, 12, 14, 16, 18, 20, 25 or more). Small fragments may form an antigenic determinant.

Such fragments may be “free-standing”, i.e. not part of or fused to other amino acids or polypeptides, or they may be comprised within a larger polypeptide of which they form a part or region. When comprised within a larger polypeptide, the fragment of the invention most preferably forms a single continuous region. For instance, certain preferred embodiments relate to a fragment having a pre- and/or pro-polypeptide region fused to the amino terminus of the fragment and/or an additional region fused to the carboxyl terminus of the fragment. However, several fragments may be comprised within a single larger polypeptide.

The polypeptides of the present invention or their immunogenic fragments (comprising at least one antigenic determinant) can be used to generate ligands, such as polyclonal or monoclonal antibodies, that are immunospecific for the polypeptides. Such antibodies may be employed to isolate or to identify clones expressing the polypeptides of the invention or to purify the polypeptides by affinity chromatography. The antibodies may also be employed as diagnostic or therapeutic aids, amongst other applications, as will be apparent to the skilled reader.

The term “immunospecific” means that the antibodies have substantially greater affinity for the polypeptide of SEQ ID NO:4 or SEQ ID NO:6 than for the known midkine family member swall|P21741|MK_HUMAN. As used herein, the term “antibody” refers to intact molecules as well as to fragments thereof, such as Fab, F(ab′)2 and Fv, which are capable of binding to the antigenic determinant in question. Such antibodies thus bind to the polypeptides of the first aspect of the invention.

By “substantially greater affinity” we mean that there is a measurable increase in the affinity for the polypeptide of SEQ ID NO:4 or SEQ ID NO:6 as compared with the affinity for the known midkine family member swall|P21741|MK_HUMAN.

Preferably, the affinity is at least 1.5-fold, 2-fold, 5-fold 10-fold, 100-fold, 10³-fold, 10⁴-fold, 10⁵-fold or 10⁶-fold greater for the polypeptide of SEQ ID NO:4 or SEQ ID NO:6 than for the known midkine family member swall|P21741|MK_HUMAN.

If polyclonal antibodies are desired, a selected mammal, such as a mouse, rabbit, goat or horse, may be immunised with a polypeptide of the first aspect of the invention. The polypeptide used to immunise the animal can be derived by recombinant DNA technology or can be synthesized chemically. If desired, the polypeptide can be conjugated to a carrier protein. Commonly used carriers to which the polypeptides may be chemically coupled include bovine serum albumin, thyroglobulin and keyhole limpet haemocyanin. The coupled polypeptide is then used to immunise the animal. Serum from the immunised animal is collected and treated according to known procedures, for example by immunoaffinity chromatography.

Monoclonal antibodies to the polypeptides of the first aspect of the invention can also be readily produced by one skilled in the art. The general methodology for making monoclonal antibodies using hybridoma technology is well known (see, for example, Kohler, G. and Milstein, C., Nature 256: 495-497 (1975); Kozbor et al., Immunology Today 4: 72 (1983); Cole et al., 77-96 in Monoclonal Antibodies and Cancer Therapy, Alan R. Liss, Inc. (1985).

Panels of monoclonal antibodies produced against the polypeptides of the first aspect of the invention can be screened for various properties, i.e., for isotype, epitope, affinity, etc. Monoclonal antibodies are particularly useful in purification of the individual polypeptides against which they are directed. Alternatively, genes encoding the monoclonal antibodies of interest may be isolated from hybridomas, for instance by PCR techniques known in the art, and cloned and expressed in appropriate vectors.

Chimeric antibodies, in which non-human variable regions are joined or fused to human constant regions (see, for example, Liu et al., Proc. Natl. Acad. Sci. USA, 84, 3439 (1987)), may also be of use.

The antibody may be modified to make it less immunogenic in an individual, for example by humanisation (see Jones et al., Nature, 321, 522 (1986); Verhoeyen et al., Science, 239, 1534 (1988); Kabat et al., J. Immunol., 147, 1709 (1991); Queen et al., Proc. Natl Acad. Sci. USA, 86, 10029 (1989); Gorman et al., Proc. Natl Acad. Sci. USA, 88, 34181 (1991); and Hodgson et al., Bio/Technology, 9, 421 (1991)). The term “humanised antibody”, as used herein, refers to antibody molecules in which the CDR amino acids and selected other amino acids in the variable domains of the heavy and/or light chains of a non-human donor antibody have been substituted in place of the equivalent amino acids in a human antibody. The humanised antibody thus closely resembles a human antibody but has the binding ability of the donor antibody.

In a further alternative, the antibody may be a “bispecific” antibody, that is an antibody having two different antigen binding domains, each domain being directed against a different epitope.

Phage display technology may be utilised to select genes which encode antibodies with binding activities towards the polypeptides of the invention either from repertoires of PCR amplified V-genes of lymphocytes from humans screened for possessing the relevant antibodies, or from naive libraries (McCafferty, J. et al., (1990), Nature 348, 552-554; Marks, J. et al., (1992) Biotechnology 10, 779-783). The affinity of these antibodies can also be improved by chain shuffling (Clackson, T. et al., (1991) Nature 352, 624-628).

Antibodies generated by the above techniques, whether polyclonal or monoclonal, have additional utility in that they may be employed as reagents in immunoassays,, radioimmunoassays (RIA) or enzyme-linked immunosorbent assays (ELISA). In these applications, the antibodies can be labelled with an analytically-detectable reagent such as a radioisotope, a fluorescent molecule or an enzyme.

Preferred nucleic acid molecules of the second and third aspects of the invention are those which encode a polypeptide sequence which comprises or consists of an amino acid sequence as recited in SEQ ID NO:2, SEQ ID NO:4, SEQ ID NO: 6, or SEQ ID NO:8, and fragments thereof and functionally equivalent polypeptides. These nucleic acid molecules may be used in the methods and applications described herein. The nucleic acid molecules of the invention preferably comprise at least n consecutive nucleotides from the sequences disclosed herein where, depending on the particular sequence, n is 10 or more (for example, 12, 14, 15, 18, 20, 25, 30, 35, 40 or more).

The nucleic acid molecules of the invention also include sequences that are complementary to nucleic acid molecules described above (for example, for antisense or probing purposes).

Nucleic acid molecules of the present invention may be in the form of RNA, such as mRNA, or in the form of DNA, including, for instance cDNA, synthetic DNA or genomic DNA. Such nucleic acid molecules may be obtained by cloning, by chemical synthetic techniques or by a combination thereof. The nucleic acid molecules can be prepared, for example, by chemical synthesis using techniques such as solid phase phosphoramidite chemical synthesis, from genomic or cDNA libraries or by separation from an organism. RNA molecules may generally be generated by the in vitro or in vivo transcription of DNA sequences.

The nucleic acid molecules may be double-stranded or single-stranded. Single-stranded DNA may be the coding strand, also known as the sense strand, or it may be the non-coding strand, also referred to as the anti-sense strand.

The term “nucleic acid molecule” also includes analogues of DNA and RNA, such as those containing modified backbones, and peptide nucleic acids (PNA). The term “PNA”, as used herein, refers to an antisense molecule or an anti-gene agent which comprises an oligonucleotide of at least five nucleotides in length linked to a peptide backbone of amino acid residues, which preferably ends in lysine. The terminal lysine confers solubility to the composition. PNAs may be pegylated to extend their lifespan in a cell, where they preferentially bind complementary single stranded DNA and RNA and stop transcript elongation (Nielsen, P. E. et al. (1993) Anticancer Drug Des. 8:53-63).

A nucleic acid molecule which encodes a polypeptide of this invention may be identical to the coding sequence of one or more of the nucleic acid molecules disclosed herein. These molecules also may have a different sequence which, as a result of the degeneracy of the genetic code, encode a polypeptide of SEQ ID NO:2, SEQ ID NO:4, SEQ ID NO: 6, or SEQ ID NO:8.

Such nucleic acid molecules may include, but are not limited to, the coding sequence for the mature polypeptide by itself; the coding sequence for the mature polypeptide and additional coding sequences, such as those encoding a leader or secretory sequence, such as a pro-, pre- or prepro-polypeptide sequence; the coding sequence of the mature polypeptide, with or without the aforementioned additional coding sequences, together with further additional, non-coding sequences, including non-coding 5′ and 3′ sequences, such as the transcribed, non-translated sequences that play a role in transcription (including termination signals), ribosome binding and mRNA stability. The nucleic acid molecules may also include additional sequences which encode additional amino acids, such as those which provide additional functionalities.

The nucleic acid molecules of the second and third aspects of the invention may also encode the fragments or the functional equivalents of the polypeptides and fragments of the first aspect of the invention. Such a nucleic acid molecule may be a naturally-occurring variant such as a naturally-occurring allelic variant, or the molecule may be a variant that is not known to occur naturally. Such non-naturally occurring variants of the nucleic acid molecule may be made by mutagenesis techniques, including those applied to nucleic acid molecules, cells or organisms.

Among variants in this regard are variants that differ from the aforementioned nucleic acid molecules by nucleotide substitutions, deletions or insertions. The substitutions, deletions or insertions may involve one or more nucleotides. The variants may be altered in coding or non-coding regions or both. Alterations in the coding regions may produce conservative or non-conservative amino acid substitutions, deletions or insertions.

The nucleic acid molecules of the invention can also be engineered, using methods generally known in the art, for a variety of reasons, including modifying the cloning, processing, and/or expression of the gene product (the polypeptide). DNA shuffling by random fragmentation and PCR reassembly of gene fragments and synthetic oligonucleotides are included as techniques which may be used to engineer the nucleotide sequences. Site-directed mutagenesis may be used to insert new restriction sites, alter glycosylation patterns, change codon preference, produce splice variants, introduce mutations and so forth.

Nucleic acid molecules which encode a polypeptide of the first aspect of the invention may be ligated to a heterologous sequence so that the combined nucleic acid molecule encodes a fusion protein. Such combined nucleic acid molecules are included within the second or third aspects of the invention. For example, to screen peptide libraries for inhibitors of the activity of the polypeptide, it may be useful to express, using such a combined nucleic acid molecule, a fusion protein that can be recognised by a commercially-available antibody. A fusion protein may also be engineered to contain a cleavage site located between the sequence of the polypeptide of the invention and the sequence of a heterologous protein so that the polypeptide may be cleaved and purified away from the heterologous protein.

The nucleic acid molecules of the invention also include antisense molecules that are partially complementary to nucleic acid molecules encoding polypeptides of the present invention and that therefore hybridise to the encoding nucleic acid molecules (hybridisation). Such antisense molecules, such as oligonucleotides, can be designed to recognise, specifically bind to and prevent transcription of a target nucleic acid encoding a polypeptide of the invention, as will be known by those of ordinary skill in the art (see, for example, Cohen, J. S., Trends in Pharm. Sci., 10, 435 (1989), Okano, J. Neurochem. 56, 560 (1991); O'Connor, J. Neurochem 56, 560 (1991); Lee et al., Nucleic Acids Res 6, 3073 (1979); Cooney et al., Science 241, 456 (1988); Dervan et al., Science 251, 1360 (1991).

The term “hybridisation” as used herein refers to the association of two nucleic acid molecules with one another by hydrogen bonding. Typically, one molecule will be fixed to a solid support and the other will be free in solution. Then, the two molecules may be placed in contact with one another under conditions that favour hydrogen bonding. Factors that affect this bonding include: the type and volume of solvent; reaction temperature; time of hybridisation; agitation; agents to block the non-specific attachment of the liquid phase molecule to the solid support (Denhardt's reagent or BLOTTO); the concentration of the molecules; use of compounds to increase the rate of association of molecules (dextran sulphate or polyethylene glycol); and the stringency of the washing conditions following hybridisation (see Sambrook et al. [supra]).

The inhibition of hybridisation of a completely complementary molecule to a target molecule may be examined using a hybridisation assay, as known in the art (see, for example, Sambrook et al [supra]). A substantially homologous molecule will then compete for and inhibit the binding of a completely homologous molecule to the target molecule under various conditions of stringency, as taught in Wahl, G. M. and S. L. Berger (1987; Methods Enzymol. 152:399-407) and Kimmel, A. R. (1987; Methods Enzymol. 152:507-511).

“Stringency” refers to conditions in a hybridisation reaction that favour the association of very similar molecules over association of molecules that differ. High stringency hybridisation conditions are defined as overnight incubation at 42° C. in a solution comprising 50% formamide, 5×SSC (150 mM NaCl, 15 mM trisodium citrate), 50 mM sodium phosphate (pH7.6), 5× Denhardts solution, 10% dextran sulphate, and 20 microgram/ml denatured, sheared salmon sperm DNA, followed b y washing the filters in 0.1×SSC at approximately 65° C. Low stringency conditions involve the hybridisation reaction being carried out at 35° C. (see Sambrook et al. [supra]). Preferably, the conditions used for hybridisation are those of high stringency.

Preferred embodiments of this aspect of the invention are nucleic acid molecules that are at least 80%, 85% or 90% identical over their entire length to a nucleic acid molecule encoding the INSP106 polypeptides and nucleic acid molecules that are substantially complementary to such nucleic acid molecules.

Preferably, a nucleic acid molecule according to this aspect of the invention comprises a region that is at least 92% identical over its entire length to such coding sequences, or is a nucleic acid molecule that is complementary thereto. In this regard, nucleic acid molecules at least 95%, preferably at least 98% or 99% identical over their entire length to the same are particularly preferred. Preferred embodiments in this respect are nucleic acid molecules that encode polypeptides which retain substantially the same biological function or activity as SEQ ID NO.4 or 6.

The invention also provides a process for detecting a nucleic acid molecule of the invention, comprising the steps of: (a) contacting a nucleic probe according to the invention with a biological sample under hybridising conditions to form duplexes; and (b) detecting any such duplexes that are formed.

As discussed additionally below in connection with assays that may be utilised according to the invention, a nucleic acid molecule as described above may be used as a hybridisation probe for RNA, cDNA or genomic DNA, in order to isolate full-length cDNAs and genomic clones encoding the INSP106 polypeptides and to isolate cDNA and genomic clones of homologous or orthologous genes that have a high sequence similarity to the gene encoding this polypeptide.

In this regard, the following techniques, among others known in the art, may be utilised and are discussed below for purposes of illustration. Methods for DNA sequencing and analysis are well known and are generally available in the art and may, indeed, be used to practice many of the embodiments of the invention discussed herein. Such methods may employ such enzymes as the Klenow fragment of DNA polymerase I, Sequenase (US Biochemical Corp, Cleveland, Ohio), Taq polymerase (Perkin Elmer), thermostable T7 polymerase (Amersham, Chicago, Ill.), or combinations of polymerases and proof-reading exonucleases such as those found in the ELONGASE Amplification System marketed by Gibco/BRL (Gaithersburg, Md.). Preferably, the sequencing process may be automated using machines such as the Hamilton Micro Lab 2200 (Hamilton, Reno, Nev.), the Peltier Thermal Cycler (PTC200; MJ Research, Watertown, Mass.) and the ABI Catalyst and 373 and 377 DNA Sequencers (Perkin Elmer).

One method for isolating a nucleic acid molecule encoding a polypeptide with an equivalent function to that of the INSP106 polypeptides is to probe a genomic or cDNA library with a natural or artificially-designed probe using standard procedures that are recognised in the art (see, for example, “Current Protocols in Molecular Biology”, Ausubel et al. (eds). Greene Publishing Association and John Wiley Interscience, New York, 1989,1992). Probes comprising at least 15, preferably at least 30, and more preferably at least 50, contiguous bases that correspond to, or are complementary to, nucleic acid sequences from SEQ ID NO:7 or SEQ ID NO:4 or 6 which nucleic acid sequences comprise at least a portion of SEQ ID NO:7) are particularly useful probes. Such probes may be labelled with an analytically-detectable reagent to facilitate their identification. Useful reagents include, but are not limited to, radioisotopes, fluorescent dyes and enzymes that are capable of catalysing the formation of a detectable product. Using these probes, the ordinarily skilled artisan will be capable of isolating complementary copies of genomic DNA, cDNA or RNA polynucleotides encoding proteins of interest from human, mammalian or other animal sources and screening such sources for related sequences, for example, for additional members of the family, type and/or subtype.

In many cases, isolated cDNA sequences will be incomplete, in that the region encoding the polypeptide will be cut short, normally at the 5′ end. Several methods are available to obtain full length cDNAs, or to extend short cDNAs. Such sequences may be extended utilising a partial nucleotide sequence and employing various methods known in the art to detect upstream sequences such as promoters and regulatory elements. For example, one method which may be employed is based on the method of Rapid Amplification of cDNA Ends (RACE; see, for example, Frohman et al., PNAS USA 85, 8998-9002, 1988). Recent modifications of this technique, exemplified by the Marathon™ technology (Clontech Laboratories Inc.), for example, have significantly simplified the search for longer cDNAs. A slightly different technique, termed “restriction-site” PCR, uses universal primers to retrieve unknown nucleic acid sequence adjacent a known locus (Sarkar, G. (1993) PCR Methods Applic. 2:318-322). Inverse PCR may also be used to amplify or to extend sequences using divergent primers based on a known region (Triglia, T. et al. (1988) Nucleic Acids Res. 16:8186). Another method which may be used is capture PCR which involves PCR amplification of DNA fragments adjacent a known sequence in human and yeast artificial chromosome DNA (Lagerstrom, M. et al. (1991) PCR Methods Applic., 1, 111-119). Another method which may be used to retrieve unknown sequences is that of Parker, J. D. et al. (1991); Nucleic Acids Res. 19:3055-3060). Additionally, one may use PCR, nested primers, and PromoterFinder™ libraries to walk genomic DNA (Clontech, Palo Alto, Calif.). This process avoids the need to screen libraries and is useful in finding intron/exon junctions.

When screening for full-length cDNAs, it is preferable to use libraries that have been size-selected to include larger cDNAs. Also, random-primed libraries are preferable, in that they will contain more sequences that contain the 5′ regions of genes. Use of a randomly primed library may be especially preferable for situations in which an oligo d(IT) library does not yield a full-length cDNA. Genomic libraries may be useful for extension of sequence into 5′ non-transcribed regulatory regions.

In one embodiment of the invention, the nucleic acid molecules of the present invention may be used for chromosome localisation. In this technique, a nucleic acid molecule is specifically targeted to, and can hybridise with, a particular location on an individual human chromosome. The mapping of relevant sequences to chromosomes according to the present invention is an important step in the confirmatory correlation of those sequences with the gene-associated disease. Once a sequence has been mapped to a precise chromosomal location, the physical position of the sequence on the chromosome can be correlated with genetic map data. Such data are found in, for example, V. McKusick, Mendelian Inheritance in Man (available on-line through Johns Hopkins University Welch Medical Library). The relationships between genes and diseases that have been mapped to the same chromosomal region are then identified through linkage analysis (coinheritance of physically adjacent genes). This provides valuable information to investigators searching for disease genes using positional cloning or other gene discovery techniques. Once the disease or syndrome has been crudely localised by genetic linkage to a particular genomic region, any sequences mapping to that area may represent associated or regulatory genes for further investigation. The nucleic acid molecule may also be used to detect differences in the chromosomal location due to translocation, inversion, etc. among normal, carrier, or affected individuals.

The nucleic acid molecules of the present invention are also valuable for tissue localisation. Such techniques allow the determination of expression patterns of the polypeptide in tissues by detection of the mRNAs that encode them. These techniques include in situ hybridisation techniques and nucleotide amplification techniques, such as PCR. Results from these studies provide an indication of the normal functions of the polypeptide in the organism. In addition, comparative studies of the normal expression pattern of mRNAs with that of mRNAs encoded by a mutant gene provide valuable insights into the role of mutant polypeptides in disease. Such inappropriate expression may be of a temporal, spatial or quantitative nature.

Gene silencing approaches may also be undertaken to down-regulate endogenous expression of a gene encoding a polypeptide of the invention. RNA interference (RNAi) (Elbashir, S M et al., Nature 2001, 411, 494498) is one method of sequence specific post-transcriptional gene silencing that may be employed. Short dsRNA oligonucleotides are synthesised in vitro and introduced into a cell. The sequence specific binding of these dsRNA oligonucleotides triggers the degradation of target mRNA, reducing or ablating target protein expression.

Efficacy of the gene silencing approaches assessed above may be assessed through the measurement of polypeptide expression (for example, by Western blotting), and at the RNA level using TaqMan-based methodologies.

The vectors of the present invention comprise nucleic acid molecules of the invention and may be cloning or expression vectors. The host cells of the invention, which may be transformed, transfected or transduced with the vectors of the invention may be prokaryotic or eukaryotic.

Preferred vectors of the invention include those set forth in the appended figures.

The polypeptides of the invention may be prepared in recombinant form by expression of their encoding nucleic acid molecules in vectors contained within a host cell. Such expression methods are well known to those of skill in the art and many are described in detail by Sambrook et al (supra) and Fernandez & Hoeffler (1998, eds. “Gene expression systems. Using nature for the art of expression”. Academic Press, San Diego, London, Boston, New York, Sydney, Tokyo, Toronto).

Generally, any system or vector that is suitable to maintain, propagate or express nucleic acid molecules to produce a polypeptide in the required host may be used. The appropriate nucleotide sequence may be inserted into an expression system by any of a variety of well-known and routine techniques, such as, for example, those described in Sambrook et al., (supra). Generally, the encoding gene can be placed under the control of a control element such as a promoter, ribosome binding site (for bacterial expression) and, optionally, an operator, so that the DNA sequence encoding the desired polypeptide is transcribed into RNA in the transformed host cell.

Examples of suitable expression systems include, for example, chromosomal, episomal and virus-derived systems, including, for example, vectors derived from: bacterial plasmids, bacteriophage, transposons, yeast episomes, insertion elements, yeast chromosomal elements, viruses such as baculoviruses, papova viruses such as SV40, vaccinia viruses, adenoviruses, fowl pox viruses, pseudorabies viruses and retroviruses, or combinations thereof, such as those derived from plasmid and bacteriophage genetic elements, including cosmids and phagemids. Human artificial chromosomes (HACs) may also be employed to deliver larger fragments of DNA than can be contained and expressed in a plasmid.

Particularly suitable expression systems include microorganisms such as bacteria transformed with recombinant bacteriophage, plasmid or cosmid DNA expression vectors; yeast transformed with yeast expression vectors; insect cell systems infected with virus expression vectors (for example, baculovirus); plant cell systems transformed with virus expression vectors (for example, cauliflower mosaic virus, CaMV; tobacco mosaic virus, TMV) or with bacterial expression vectors (for example, Ti or pBR322 plasmids); or animal cell systems. Cell-free translation systems can also be employed to produce the polypeptides of the invention.

Introduction of nucleic acid molecules encoding a polypeptide of the present invention into host cells can be effected by methods described in many standard laboratory manuals, such as Davis et al., Basic Methods in Molecular Biology (1986) and Sambrook et al.,(supra). Particularly suitable methods include calcium phosphate transfection, DEAE-dextran mediated transfection, transvection, microinjection, cationic lipid-mediated transfection, electroporation, transduction, scrape loading, ballistic introduction or infection (see Sambrook et al., 1989 [supra]; Ausubel et al., 1991 [supra]; Spector, Goldman & Leinwald, 1998). In eukaryotic cells, expression systems may either be transient (for example, episomal) or permanent (chromosomal integration) according to the needs of the system.

The encoding nucleic acid molecule may or may not include a sequence encoding a control sequence, such as a signal peptide or leader sequence, as desired, for example, for secretion of the translated polypeptide into the lumen of the endoplasmic reticulum, into the periplasmic space or into the extracellular environment. These signals may be endogenous to the polypeptide or they may be heterologous signals. Leader sequences can be removed by the bacterial host in post-translational processing.

In addition to control sequences, it may be desirable to add regulatory sequences that allow for regulation of the expression of the polypeptide relative to the growth of the host cell. Examples of regulatory sequences are those which cause the expression of a gene to be increased or decreased in response to a chemical or physical stimulus, including the presence of a regulatory compound or to various temperature or metabolic conditions. Regulatory sequences are those non-translated regions of the vector, such as enhancers, promoters and 5′ and 3′ untranslated regions. These interact with host cellular proteins to carry out transcription and translation. Such regulatory sequences may vary in their strength and specificity. Depending on the vector system and host utilised, any number of suitable transcription and translation elements, including constitutive and inducible promoters, may be used. For example, when cloning in bacterial systems, inducible promoters such as the hybrid lacZ promoter of the Bluescript phagemid (Stratagene, LaJolla, Calif.) or pSportl™ plasmid (Gibco BRL) and the like may be used. The baculovirus polyhedrin promoter may be used in insect cells. Promoters or enhancers derived from the genomes of plant cells (for example, heat shock, RUBISCO and storage protein genes) or from plant viruses (for example, viral promoters or leader sequences) may be cloned into the vector. In mammalian cell systems, promoters from mammalian genes or from mammalian viruses are preferable. If it is necessary to generate a cell line that contains multiple copies of the sequence, vectors based on SV40 or EBV may be used with an appropriate selectable marker.

An expression vector is constructed so that the particular nucleic acid coding sequence is located in the vector with the appropriate regulatory sequences, the positioning and orientation of the coding sequence with respect to the regulatory sequences being such that the coding sequence is transcribed under the “control” of the regulatory sequences, i.e., RNA polymerase which binds to the DNA molecule at the control sequences transcribes the coding sequence. In some cases it may be necessary to modify the sequence so that it may be attached to the control sequences with the appropriate orientation; i.e., to maintain the reading frame.

The control sequences and other regulatory sequences may be ligated to the nucleic acid coding sequence prior to insertion into a vector. Alternatively, the coding sequence can be cloned directly into an expression vector that already contains the control sequences and an appropriate restriction site.

For long-term, high-yield production of a recombinant polypeptide, stable expression is preferred. For example, cell lines which stably express the polypeptide of interest may be transformed using expression vectors which may contain viral origins of replication and/or endogenous expression elements and a selectable marker gene on the same or on a separate vector. Following the introduction of the vector, cells may be allowed to grow for 1-2 days in an enriched media before they are switched to selective media. The purpose of the selectable marker is to confer resistance to selection, and its presence allows growth and recovery of cells that successfully express the introduced sequences. Resistant clones of stably transformed cells may be proliferated using tissue culture techniques appropriate to the cell type.

Mammalian cell lines available as hosts for expression are known in the art and include many immortalised cell lines available from the American Type Culture Collection (ATCC) including, but not limited to, Chinese hamster ovary (CHO), HeLa, baby hamster kidney (BHK), monkey kidney (COS), C127, 3T3, BHK, HEK 293, Bowes melanoma and human hepatocellular carcinoma (for example Hep G2) cells and a number of other cell lines.

In the baculovirus system, the materials for baculovirus/insect cell expression systems are commercially available in kit form from, inter alia, Invitrogen, San Diego Calif. (the “MaxBac” kit). These techniques are generally known to those skilled in the art and are described fully in Summers and Smith, Texas Agricultural Experiment Station Bulletin No. 1555 (1987). Particularly suitable host cells for use in this system include insect cells such as Drosophila S2 and Spodoptera Sf9 cells.

There are many plant cell culture and whole plant genetic expression systems known in the art. Examples of suitable plant cellular genetic expression systems include those described in U.S. Pat. No. 5,693,506; U.S. Pat. No. 5,659,122; and U.S. Pat. No. 5,608,143. Additional examples of genetic expression in plant cell culture has been described by Zenk, Phytochemistry 30, 3861-3863 (1991).

In particular, all plants from which protoplasts can be isolated and cultured to give whole regenerated plants can be utilised, so that whole plants are recovered which contain the transferred gene. Practically all plants can be regenerated from cultured cells or tissues, including but not limited to all major species of sugar cane, sugar beet, cotton, fruit and other trees, legumes and vegetables.

Examples of particularly preferred bacterial host cells include streptococci, staphylococci, E. coli, Streptomyces and Bacillus subtilis cells.

Examples of particularly suitable host cells for fungal expression include yeast cells (for example, S. cerevisiae) and Aspergillus cells.

Any number of selection systems are known in the art that may be used to recover transformed cell lines. Examples include the herpes simplex virus thymidine kinase (Wigler, M. et al. (1977) Cell 11:223-32) and adenine phosphoribosyltransferase (Lowy, I. et al. (1980) Cell 22:817-23) genes that can be employed in tk⁻ or aprt^{±} cells, respectively.

Also, antimetabolite, antibiotic or herbicide resistance can be used as the basis for selection; for example, dihydrofolate reductase (DHFR) that confers resistance to methotrexate (Wigler, M. et al. (1980) Proc. Natl. Acad. Sci. 77:3567-70); npt, which confers resistance to the aminoglycosides neomycin and G418 (Colbere-Garapin, F. et al (1981) J. Mol. Biol. 150:1-14) and als or pat, which confer resistance to chlorsulfuron and phosphinotricin acetyltransferase, respectively. Additional selectable genes have been described, examples of which will be clear to those of skill in the art.

Although the presence or absence of marker gene expression suggests that the gene of interest is also present, its presence and expression may need to be confirmed. For example, if the relevant sequence is inserted within a marker gene sequence, transformed cells containing the appropriate sequences can be identified by the absence of marker gene function. Alternatively, a marker gene can be placed in tandem with a sequence encoding a polypeptide of the invention under the control of a single promoter. Expression of the marker gene in response to induction or selection usually indicates expression of the tandem gene as well.

Alternatively, host cells that contain a nucleic acid sequence encoding a polypeptide of the invention and which express said polypeptide may be identified by a variety of procedures known to those of skill in the art. These procedures include, but are not limited to, DNA-DNA or DNA-RNA hybridisations and protein bioassays, for example, fluorescence activated cell sorting (FACS) or immunoassay techniques (such as the enzyme-linked immunosorbent assay [ELISA] and radioimmunoassay [RIA]), that include membrane, solution, or chip based technologies for the detection and/or quantification of nucleic acid or protein (see Hampton, R. et al. (1990) Serological Methods, a Laboratory Manual, APS Press, St Paul, Minn.) and Maddox, D. E. et al. (1983) J. Exp. Med, 158, 1211-1216).

A wide variety of labels and conjugation techniques are known by those skilled in the art and may be used in various nucleic acid and amino acid assays. Means for producing labelled hybridisation or PCR probes for detecting sequences related to nucleic acid molecules encoding polypeptides of the present invention include oligolabelling, nick translation, end-labelling or PCR amplification using a labelled polynucleotide. Alternatively, the sequences encoding the polypeptide of the invention may be cloned into a vector for the production of an mRNA probe. Such vectors are known in the art, are commercially available, and may be used to synthesise RNA probes in vitro by addition of an appropriate RNA polymerase such as T7, T3 or SP6 and labelled nucleotides. These procedures may be conducted using a variety of commercially available kits (Pharmacia & Upjohn, (Kalamazoo, Mich.); Promega (Madison Wis.); and U.S. Biochemical Corp., Cleveland, Ohio)).

Suitable reporter molecules or labels, which may be used for ease of detection, include radionuclides, enzymes and fluorescent, chemiluminescent or chromogenic agents as well as substrates, cofactors, inhibitors, magnetic particles, and the like.

Nucleic acid molecules according to the present invention may also be used to create transgenic animals, particularly rodent animals. Such transgenic animals form a further aspect of the present invention. This may be done locally by modification of somatic cells, or by germ line therapy to incorporate heritable modifications. Such transgenic animals may be particularly useful in the generation of animal models for drug molecules effective as modulators of the polypeptides of the present invention.

The polypeptide can be recovered and purified from recombinant cell cultures by well-known methods including ammonium sulphate or ethanol precipitation, acid extraction, anion or cation exchange chromatography, phosphocellulose chromatography, hydrophobic interaction chromatography, affinity chromatography, hydroxylapatite chromatography and lectin chromatography. High performance liquid chromatography is particularly useful for purification. Well known techniques for refolding proteins may be employed to regenerate an active conformation when the polypeptide is denatured during isolation and or purification.

Specialised vector constructions may also be used to facilitate purification of proteins, as desired, by joining sequences encoding the polypeptides of the invention to a nucleotide sequence encoding a polypeptide domain that will facilitate purification of soluble proteins. Examples of such purification-facilitating domains include metal chelating peptides such as histidine-tryptophan modules that allow purification on immobilised metals, protein A domains that allow purification on immobilised immunoglobulin, and the domain utilised in the FLAGS extension/affinity purification system (Inmunex Corp., Seattle, Wash.). The inclusion of cleavable linker sequences such as those specific for Factor XA or enterokinase (Invitrogen, San Diego, Calif.) between the purification domain and the polypeptide of the invention may be used to facilitate purification. One such expression vector provides for expression of a fusion protein containing the polypeptide of the invention fused to several histidine residues preceding a thioredoxin or an enterolinase cleavage site. The histidine residues facilitate purification by IMAC (immobilised metal ion affinity chromatography as described in Porath, J. et al. (1992), Prot. Exp. Purif. 3: 263-281) while the thioredoxin or enterokinase cleavage site provides a means for purifying the polypeptide from the fusion protein. A discussion of vectors which contain fusion proteins is provided in Kroll, D. J. et al. (1993; DNA Cell Biol. 12:441-453).

If the polypeptide is to be expressed for use in screening assays, generally it is preferred that it be produced at the surface of the host cell in which it is expressed. In this event, the host cells may be harvested prior to use in the screening assay, for example using techniques such as fluorescence activated cell sorting (FACS) or immunoaffinity techniques. If the polypeptide is secreted into the medium, the medium can be recovered in order to recover and purify the expressed polypeptide. If polypeptide is produced intracellularly, the cells must first be lysed before the polypeptide is recovered.

The polypeptide of the invention can be used to screen libraries of compounds in any of a variety of drug screening techniques. Such compounds may activate (agonise) or inhibit (antagonise) the level of expression of the gene or the activity of the polypeptide of the invention and form a further aspect of the present invention. Preferred compounds are effective to alter the expression of a natural gene which encodes a polypeptide of the first aspect of the invention or to regulate the activity of a polypeptide of the first aspect of the invention.

Agonist or antagonist compounds may be isolated from, for example, cells, cell-free preparations, chemical libraries or natural product mixtures. These agonists or antagonists may be natural or modified substrates, ligands, enzymes, receptors or structural or functional mimetics. For a suitable review of such screening techniques, see Coligan et al., Current Protocols in Immunology 1(2):Chapter 5 (1991).

Compounds that are most likely to be good antagonists are molecules that bind to the polypeptide of the invention without inducing the biological effects of the polypeptide upon binding to it. Potential antagonists include small organic molecules, peptides, polypeptides and antibodies that bind to the polypeptide of the invention and thereby inhibit or extinguish its activity. In this fashion, binding of the polypeptide to normal cellular binding molecules may be inhibited, such that the normal biological activity of the polypeptide is prevented.

The polypeptide of the invention that is employed in such a screening technique may be free in solution, affixed to a solid support, borne on a cell surface or located intracellularly. In general, such screening procedures may involve using appropriate cells or cell membranes that express the polypeptide that are contacted with a test compound to observe binding, or stimulation or inhibition of a functional response. The functional response of the cells contacted with the test compound is then compared with control cells that were not contacted with the test compound. Such an assay may assess whether the test compound results in a signal generated by activation of the polypeptide, using an appropriate detection system. Inhibitors of activation are generally assayed in the presence of a known agonist and the effect on activation by the agonist in the presence of the test compound is observed.

A preferred method for identifying an agonist or antagonist compound of a polypeptide of the present invention comprises:

(a) contacting a cell expressing on the surface thereof the polypeptide according to the first aspect of the invention, the polypeptide being associated with a second component capable of providing a detectable signal in response to the binding of a compound to the polypeptide, with a compound to be screened under conditions to permit binding to the polypeptide; and

(b) determining whether the compound binds to and activates or inhibits the polypeptide by measuring the level of a signal generated from the interaction of the compound with the polypeptide.

Methods for generating detectable signals in the types of assays described herein will be known to those of skill in the art. A particular example is cotransfecting a construct expressing a polypeptide according to the invention, or a fragment such as the LBD, in fusion with the GAL4 DNA binding domain, into a cell together with a reporter plasmid, an example of which is pFR-Luc (Stratagene Europe, Amsterdam, The Netherlands). This particular plasmid contains a synthetic promoter with five tandem repeats of GAL4 binding sites that control the expression of the luciferase gene. When a potential ligand is added to the cells, it will bind the GAL4-polypeptide fusion and induce transcription of the luciferase gene. The level of the luciferase expression can be monitored by its activity using a luminescence reader (see, for example, Lehman et al JBC 270, 12953, 1995; Pawar et al JBC, 277, 39243, 2002).

A further preferred method for identifying an agonist or antagonist of a polypeptide of the invention comprises:

(a) contacting a cell expressing on the surface thereof the polypeptide, the polypeptide being associated with a second component capable of providing a detectable signal in response to the binding of a compound to the polypeptide, with a compound to be screened under conditions to permit binding to the polypeptide; and

(b) determining whether the compound binds to and activates or inhibits the polypeptide by comparing the level of a signal generated from the interaction of the compound with the polypeptide with the level of a signal in the absence of the compound.

In further preferred embodiments, the general methods that are described above may further comprise conducting the identification of agonist or antagonist in the presence of labelled or unlabelled ligand for the polypeptide.

In another embodiment of the method for identifying agonist or antagonist of a polypeptide of the present invention comprises:

determining the inhibition of binding of a ligand to cells which have a polypeptide of the invention on the surface thereof, or to cell membranes containing such a polypeptide, in the presence of a candidate compound under conditions to permit binding to the polypeptide, and determining the amount of ligand bound to the polypeptide. A compound capable of causing reduction of binding of a ligand is considered to be an agonist or antagonist. Preferably the ligand is labelled.

More particularly, a method of screening for a polypeptide antagonist or agonist compound comprises the steps of:

(a) incubating a labelled ligand with a whole cell expressing a polypeptide according to the invention on the cell surface, or a cell membrane containing a polypeptide of the invention,

(b) measuring the amount of labelled ligand bound to the whole cell or the cell membrane;

(c) adding a candidate compound to a mixture of labelled ligand and the whole cell or the cell membrane of step (a) and allowing the mixture to attain equilibrium;

(d) measuring the amount of labelled ligand bound to the whole cell or the cell membrane after step (c); and

(e) comparing the difference in the labelled ligand bound in step (b) and (d), such that the compound which causes the reduction in binding in step (d) is considered to be an agonist or antagonist.

The INSP106 polypeptides of the present invention may modulate a variety of physiological and pathological processes, including processes such as cellular proliferation and migration within the immune system. The polypeptides of the invention may be used for the modulation of (e.g. the antagonisation of) neurite growth, neuronal cell survival, plasminogen activation, heparin-binding, the maintenance and differentiation of embryonic nerve cells, the maintenance and differentiation of ES cell lines, to regulate embryonic development, dentition and tissue repair. Thus, the biological activity of the INSP106 polypeptides can be examined in systems that allow the study of such modulatory activities, using a variety of suitable assays. A suitable assay is described by Akhter, S., Tanaka, I. T., Kojima, S., Muramatsu, H., Inui, T., Kimura, T., Kaneda, N., Talukder, A. H., Muramatsu T (1998) Clusters of Basic Amino Acids in Midkine: Roles in Neurite-Promoting Activity and Plasminogen Activator-Enhancing Activity. J. Biochem. 123, 1127-1136, and references therein.

For example, for observing cell growth inhibition, one can use a solid or liquid medium. In a solid medium, cells undergoing growth inhibition can easily be selected from the subject cell group by comparing the sizes of colonies formed. In a liquid medium, growth inhibition can be screened by measuring culture medium turbity or incorporation of labelled thymidine in DNA. Typically, the incorporation of a nucleoside analog into newly synthesised DNA may be employed to measure proliferation (i. e., active cell growth) in a population of cells. For example, bromodeoxyuridine (BrdU) can be employed as a DNA labelling reagent and anti-BrdU mouse monoclonal antibodies can be employed as a detection reagent. This antibody binds only to cells containing DNA which has incorporated bromodeoxyuridine. A number of detection methods may be used in conjunction with this assay including immunofluorescence, immunohistochemical, ELISA, and colorimetric methods. Kits that include bromodeoxyuridine (BrdU) and anti-BrdU mouse monoclonal antibody are commercially available from Boehringer Mannheim (Indianapolis, Ind.).

The INSP106 polypeptides may be found to modulate a variety of physiological and pathological processes in a dose-dependent manner in the above-described assays. Thus, the “functional equivalents” of the INSP106 polypeptides include polypeptides that exhibit any of the same modulatory activities in the above-described assays in a dose-dependent manner. Although the degree of dose-dependent activity need not be identical to that of the INSP106 polypeptides, preferably the “functional equivalents” will exhibit substantially similar dose-dependence in a given activity assay compared to the INSP106 polypeptides.

In certain of the embodiments described above, simple binding assays may be used, in which the adherence of a test compound to a surface bearing the polypeptide is detected by means of a label directly or indirectly associated with the test compound or in an assay involving competition with a labelled competitor. In another embodiment, competitive drug screening assays may be used, in which neutralising antibodies that are capable of binding the polypeptide specifically compete with a test compound for binding. In this manner, the antibodies can be used to detect the presence of any test compound that possesses specific binding affinity for the polypeptide.

Assays may also be designed to detect the effect of added test compounds on the production of mRNA encoding the polypeptide in cells. For example, an ELISA may be constructed that measures secreted or cell-associated levels of polypeptide using monoclonal or polyclonal antibodies by standard methods known in the art, and this can be used to search for compounds that may inhibit or enhance the production of the polypeptide from suitably manipulated cells or tissues. The formation of binding complexes between the polypeptide and the compound being tested may then be measured.

Assay methods that are also included within the terms of the present invention are those that involve the use of the genes and polypeptides of the invention in overexpression or ablation assays. Such assays involve the manipulation of levels of these genes/polypeptides in cells and assessment of the impact of this manipulation event on the physiology of the manipulated cells. For example, such experiments reveal details of signalling and metabolic pathways in which the particular genes/polypeptides are implicated, generate information regarding the identities of polypeptides with which the studied polypeptides interact and provide clues as to methods by which related genes and proteins are regulated.

Another technique for drug screening which may be used provides for high throughput screening of compounds having suitable binding affinity to the polypeptide of interest (see International patent application WO84/03564). In this method, large numbers of different small test compounds are synthesised on a solid substrate, which may then be reacted with the polypeptide of the invention and washed. One way of immobilising the polypeptide is to use non-neutralising antibodies. Bound polypeptide may then be detected using methods that are well known in the art. Purified polypeptide can also be coated directly onto plates for use in the aforementioned drug screening techniques.

The polypeptide of the invention may be used to identify membrane-bound or soluble receptors, through standard receptor binding techniques that are known in the art, such as ligand binding and crosslinking assays in which the polypeptide is labelled with a radioactive isotope, is chemically modified, or is fused to a peptide sequence that facilitates its detection or purification, and incubated with a source of the putative receptor (for example, a composition of cells, cell membranes, cell supernatants, tissue extracts, or bodily fluids). The efficacy of binding may be measured using biophysical techniques such as surface plasmon resonance and spectroscopy. Binding assays may be used for the purification and cloning of the receptor, but may also identify agonists and antagonists of the polypeptide, that compete with the binding of the polypeptide to its receptor. Standard methods for conducting screening assays are well understood in the art.

The INSP106 polypeptides of the present invention may modulate a variety of physiological and pathological processes, including processes such as cellular growth and cellular metastasis (including cancer cell metastasis). Thus, the biological activity of the INSP106 polypeptides can be examined in systems that allow the study of such modulatory activities, using a variety of suitable assays.

For example, for observing the effect of the INSP106 polypeptides of the present invention on cellular metastasis, one can employ one or more of the methods described in Ohtaki et al., Nature. 2001 May 31;411(6837):613-7 or the publications referred to therein.

The INSP 106 polypeptides of the present invention may also be used for the identification and characterisation of receptors which interact with the INSP106 polypeptides of the present invention. Suitable methods of identification and characterisation include, but are not limited to, those described in Hinuma et al., Nat Cell Biol. 2000 October;2(10):703-8 and the International patent application published as WO01/17958 or the publications referred to therein.

The INSP106 polypeptides may be found to modulate a variety of physiological and pathological processes in a dose-dependent manner in the above-described assays. Thus, the “functional equivalents” of the INSP106 polypeptides include polypeptides that exhibit any of the same modulatory activities in the above-described assays in a dose-dependent manner. Although the degree of dose-dependent activity need not be identical to that of the INSP106 polypeptides, preferably the “functional equivalents” will exhibit substantially similar dose-dependence in a given activity assay compared to the INSP106 polypeptides.

The invention also includes a screening kit useful in the methods for identifying agonists, antagonists, ligands, receptors, substrates, enzymes, that are described above.

The invention includes the agonists, antagonists, ligands, receptors, substrates and enzymes, and other compounds which modulate the activity or antigenicity of the polypeptide of the invention discovered by the methods that are described above.

The invention also provides pharmaceutical compositions comprising a polypeptide, nucleic acid, ligand or compound of the invention in combination with a suitable pharmaceutical carrier. These compositions may be suitable as therapeutic or diagnostic reagents, as vaccines, or as other immunogenic compositions, as outlined in detail below.

According to the terminology used herein, a composition containing a polypeptide, nucleic acid, ligand or compound [X] is “substantially free of” impurities [herein, Y] when at least 85% by weight of the total X+Y in the composition is X. Preferably, X comprises at least about 90% by weight of the total of X+Y in the composition, more preferably at least about 95%, 98% or even 99% by weight.

The pharmaceutical compositions should preferably comprise a therapeutically effective amount of the polypeptide, nucleic acid molecule, ligand, or compound of the invention. The term “therapeutically effective amount” as used herein refers to an amount of a therapeutic agent needed to treat, ameliorate, or prevent a targeted disease or condition, or to exhibit a detectable therapeutic or preventative effect. For any compound, the therapeutically effective dose can be estimated initially either in cell culture assays, for example, of neoplastic cells, or in animal models, usually mice, rabbits, dogs, or pigs. The animal model may also be used to determine the appropriate concentration range and route of administration. Such information can then be used to determine useful doses and routes for administration in humans.

The precise effective amount for a human subject will depend upon the severity of the disease state, general health of the subject, age, weight, and gender of the subject, diet, time and frequency of administration, drug combination(s), reaction sensitivities, and tolerance/response to therapy. This amount can be determined by routine experimentation and is within the judgement of the clinician. Generally, an effective dose will be from 0.01 mg/kg to 50 mg/kg, preferably 0.05 mg/kg to 10 mg/kg. Compositions may be administered individually to a patient or may be administered in combination with other agents, drugs or hormones.

A pharmaceutical composition may also contain a pharmaceutically acceptable carrier, for administration of a therapeutic agent. Such carriers include antibodies and other polypeptides, genes and other therapeutic agents such as liposomes, provided that the carrier does not itself induce the production of antibodies harmful to the individual receiving the composition, and which may be administered without undue toxicity. Suitable carriers may be large, slowly metabolised macromolecules such as proteins, polysaccharides, polylactic acids, polyglycolic acids, polymeric amino acids, amino acid copolymers and inactive virus particles.

Pharmaceutically acceptable salts can be used therein, for example, mineral acid salts such as hydrochlorides, hydrobromides, phosphates, sulphates, and the like; and the salts of organic acids such as acetates, propionates, malonates, benzoates, and the like. A thorough discussion of pharmaceutically acceptable carriers is available in Remington's Pharmaceutical Sciences (Mack Pub. Co., N.J. 1991).

Pharmaceutically acceptable carriers in therapeutic compositions may additionally contain liquids such as water, saline, glycerol and ethanol. Additionally, auxiliary substances, such as wetting or emulsifying agents, pH buffering substances, and the like, may be present in such compositions. Such carriers enable the pharmaceutical compositions to be formulated as tablets, pills, dragees, capsules, liquids, gels, syrups, slurries, suspensions, and the like, for ingestion by the patient.

Once formulated, the compositions of the invention can be administered directly to the subject. The subjects to be treated can be animals; in particular, human subjects can be treated.

The pharmaceutical compositions utilised in this invention may be administered by any number of routes including, but not limited to, oral, intravenous, intramuscular, intra-arterial, intramedullary, intrathecal, intraventricular, transdermal or transcutaneous applications (for example, see WO98/20734), subcutaneous, intraperitoneal, intranasal, enteral, topical, sublingual, intravaginal or rectal means. Gene guns or hyposprays may also be used to administer the pharmaceutical compositions of the invention. Typically, the therapeutic compositions may be prepared as injectables, either as liquid solutions or suspensions; solid forms suitable for solution in, or suspension in, liquid vehicles prior to injection may also be prepared.

Direct delivery of the compositions will generally be accomplished by injection, subcutaneously, intraperitoneally, intravenously or intramuscularly, or delivered to the interstitial space of a tissue. The compositions can also be administered into a lesion. Dosage treatment may be a single dose schedule or a multiple dose schedule.

If the activity of the polypeptide of the invention is in excess in a particular disease state, several approaches are available. One approach comprises administering to a subject an inhibitor compound (antagonist) as described above, along with a pharmaceutically acceptable carrier in an amount effective to inhibit the function of the polypeptide, such as by blocking the binding of ligands, substrates, enzymes, receptors, or by inhibiting a second signal, and thereby alleviating the abnormal condition. Preferably, such antagonists are antibodies. Most preferably, such antibodies are chimeric and/or humanised to minimise their immunogenicity, as described previously.

In another approach, soluble forms of the polypeptide that retain binding affinity for the ligand, substrate, enzyme, receptor, in question, may be administered. Typically, the polypeptide may be administered in the form of fragments that retain the relevant portions.

In an alternative approach, expression of the gene encoding the polypeptide can be inhibited using expression blocking techniques, such as the use of antisense nucleic acid molecules (as described above), either internally generated or separately administered. Modifications of gene expression can be obtained by designing complementary sequences or antisense molecules (DNA, RNA, or PNA) to the control, 5′ or regulatory regions (signal sequence, promoters, enhancers and introns) of the gene encoding the polypeptide. Similarly, inhibition can be achieved using “triple helix” base-pairing methodology. Triple helix pairing is useful because it causes inhibition of the ability of the double helix to open sufficiently for the binding of polymerases, transcription factors, or regulatory molecules. Recent therapeutic advances using triplex DNA have been described in the literature (Gee, J. E. et al. (1994) In: Huber, B. E. and B. I. Carr, Molecular and Immunologic Approaches, Futura Publishing Co., Mt. Kisco, N.Y.). The complementary sequence or antisense molecule may also be designed to block translation of mRNA by preventing the transcript from binding to ribosomes. Such oligonucleotides may be administered or may be generated in situ from expression in vivo.

In addition, expression of the polypeptide of the invention may be prevented by using ribozymes specific to its encoding mRNA sequence. Ribozymes are catalytically active RNAs that can be natural or synthetic (see for example Usman, N, et al., Curr. Opin. Struct. Biol (1996) 6(4), 527-33). Synthetic ribozymes can be designed to specifically cleave mRNAs at selected positions thereby preventing translation of the mRNAs into functional polypeptide. Ribozymes may be synthesised with a natural ribose phosphate backbone and natural bases, as normally found in RNA molecules. Alternatively the ribozymes may be synthesised with non-natural backbones, for example, 2′-O-methyl RNA, to provide protection from ribonuclease degradation and may contain modified bases.

RNA molecules may be modified to increase intracellular stability and half-life. Possible modifications include, but are not limited to, the addition of flanking sequences at the 5′ and/or 3′ ends of the molecule or the use of phosphorothioate or 2′ O-methyl rather than phosphodiesterase linkages within the backbone of the molecule. This concept is inherent in the production of PNAs and can be extended in all of these molecules by the inclusion of non-traditional bases such as inosine, queosine and butosine, as well as acetyl-, methyl-, thio- and similarly modified forms of adenine, cytidine, guanine, thymine and uridine which are not as easily recognised by endogenous endonucleases.

For treating abnormal conditions related to an under-expression of the polypeptide of the invention and its activity, several approaches are also available. One approach comprises administering to a subject a therapeutically effective amount of a compound that activates the polypeptide, i.e., an agonist as described above, to alleviate the abnormal condition. Alternatively, a therapeutic amount of the polypeptide in combination with a suitable pharmaceutical carrier may be administered to restore the relevant physiological balance of polypeptide.

Gene therapy may be employed to effect the endogenous production of the polypeptide by the relevant cells in the subject. Gene therapy is used to treat permanently the inappropriate production of the polypeptide by replacing a defective gene with a corrected therapeutic gene.

Gene therapy of the present invention can occur in vivo or ex vivo. Ex vivo gene therapy requires the isolation and purification of patient cells, the introduction of a therapeutic gene and introduction of the genetically altered cells back into the patient. In contrast, in vivo gene therapy does not require isolation and purification of a patient's cells.

The therapeutic gene is typically “packaged” for administration to a patient. Gene delivery vehicles may be non-viral, such as liposomes, or replication-deficient viruses, such as adenovirus as described by Berkner, K. L., in Curr. Top. Microbiol. Immunol., 158, 39-66 (1992) or adeno-associated virus (AAV) vectors as described by Muzyczka, N., in Curr. Top. Microbiol. Immunol., 158, 97-129 (1992) and U.S. Pat. No. 5,252,479. For example, a nucleic acid molecule encoding a polypeptide of the invention may be engineered for expression in a replication-defective retroviral vector. This expression construct may then be isolated and introduced into a packaging cell transduced with a retroviral plasmid vector containing RNA encoding the polypeptide, such that the packaging cell now produces infectious viral particles containing the gene of interest. These producer cells may be administered to a subject for engineering cells in vivo and expression of the polypeptide in vivo (see Chapter 20, Gene Therapy and other Molecular Genetic-based Therapeutic Approaches, (and references cited therein) in Human Molecular Genetics (1996), T Strachan and A P Read, BIOS Scientific Publishers Ltd).

Another approach is the administration of “naked DNA” in which the therapeutic gene is directly injected into the bloodstream or muscle tissue.

In situations in which the polypeptides or nucleic acid molecules of the invention are disease-causing agents, the invention provides that they can be used in vaccines to raise antibodies against the disease causing agent.

Vaccines according to the invention may either be prophylactic (ie. to prevent infection) or therapeutic (ie. to treat disease after infection). Such vaccines comprise immunising antigen(s), immunogen(s), polypeptide(s), protein(s) or nucleic acid, usually in combination with pharmaceutically-acceptable carriers as described above, which include any carrier that does not itself induce the production of antibodies harmful to the individual receiving the composition. Additionally, these carriers may function as immunostimulating agents (“adjuvants”). Furthermore, the antigen or immunogen may be conjugated to a bacterial toxoid, such as a toxoid from diphtheria, tetanus, cholera, H. pylori, and other pathogens.

Since polypeptides may be broken down in the stomach, vaccines comprising polypeptides are preferably administered parenterally (for instance, subcutaneous, intramuscular, intravenous, or intradermal injection). Formulations suitable for parenteral administration include aqueous and non-aqueous sterile injection solutions which may contain anti-oxidants, buffers, bacteriostats and solutes which render the formulation isotonic with the blood of the recipient, and aqueous and non-aqueous sterile suspensions which may include suspending agents or thickening agents.

The vaccine formulations of the invention may be presented in unit-dose or multi-dose containers. For example, sealed ampoules and vials and may be stored in a freeze-dried condition requiring only the addition of the sterile liquid carrier immediately prior to use. The dosage will depend on the specific activity of the vaccine and can be readily determined by routine experimentation.

Genetic delivery of antibodies that bind to polypeptides according to the invention may also be effected, for example, as described in International patent application WO98/55607.

The technology referred to as jet injection (see, for example, www.powderject.com) may also be useful in the formulation of vaccine compositions.

A number of suitable methods for vaccination and vaccine delivery systems are described in International patent application WO00/29428.

This invention also relates to the use of nucleic acid molecules according to the present invention as diagnostic reagents. Detection of a mutated form of the gene characterised by the nucleic acid molecules of the invention which is associated with a dysfunction will provide a diagnostic tool that can add to, or define, a diagnosis of a disease, or susceptibility to a disease, which results from under-expression, over-expression or altered spatial or temporal expression of the gene. Individuals carrying mutations in the gene may be detected at the DNA level by a variety of techniques.

Nucleic acid molecules for diagnosis may be obtained from a subject's cells, such as from blood, urine, saliva, tissue biopsy or autopsy material. The genomic DNA may be used directly for detection or may be amplified enzymatically by using PCR, ligase chain reaction (LCR), strand displacement amplification (SDA), or other amplification techniques (see Saiki et al., Nature, 324, 163-166 (1986); Bej, et al., Crit. Rev. Biochem. Molec. Biol., 26, 301-334 (1991); Birkenmeyer et al., J. Virol. Meth., 35, 117-126 (1991); Van Brunt, J., Bio/Technology, 8, 291-294 (1990)) prior to analysis.

In one embodiment, this aspect of the invention provides a method of diagnosing a disease in a patient, comprising assessing the level of expression of a natural gene encoding a polypeptide according to the invention and comparing said level of expression to a control level, wherein a level that is different to said control level is indicative of disease. The method may comprise the steps of:
a)contacting a sample of tissue from the patient with a nucleic acid probe under stringent conditions that allow the formation of a hybrid complex between a nucleic acid molecule of the invention and the probe;
b)contacting a control sample with said probe under the same conditions used in step a);
c)and detecting the presence of hybrid complexes in said samples;

wherein detection of levels of the hybrid complex in the patient sample that differ from levels of the hybrid complex in the control sample is indicative of disease.

A further aspect of the invention comprises a diagnostic method comprising the steps of:
a)obtaining a tissue sample from a patient being tested for disease;
b)isolating a nucleic acid molecule according to the invention from said tissue sample; and
c)diagnosing the patient for disease by detecting the presence of a mutation in the nucleic acid molecule which is associated with disease.

To aid the detection of nucleic acid molecules in the above-described methods, an amplification step, for example using PCR, may be included.

Deletions and insertions can be detected by a change in the size of the amplified product in comparison to the normal genotype. Point mutations can be identified by hybridising amplified DNA to labelled RNA of the invention or alternatively, labelled antisense DNA sequences of the invention. Perfectly-matched sequences can be distinguished from mismatched duplexes by RNase digestion or by assessing differences in melting temperatures. The presence or absence of the mutation in the patient may be detected by contacting DNA with a nucleic acid probe that hybridises to the DNA under stringent conditions to form a hybrid double-stranded molecule, the hybrid double-stranded molecule having an unhybridised portion of the nucleic acid probe strand at any portion corresponding to a mutation associated with disease; and detecting the presence or absence of an unhybridised portion of the probe strand as an indication of the presence or absence of a disease-associated mutation in the corresponding portion of the DNA strand.

Such diagnostics are particularly useful for prenatal and even neonatal testing.

Point mutations and other sequence differences between the reference gene and “mutant” genes can be identified by other well-known techniques, such as direct DNA sequencing or single-strand conformational polymorphism, (see Orita et al., Genomics, 5, 874-879 (1989)). For example, a sequencing primer may be used with double-stranded PCR product or a single-stranded template molecule generated by a modified PCR. The sequence determination is performed by conventional procedures with radiolabelled nucleotides or by automatic sequencing procedures with fluorescent-tags. Cloned DNA segments may also be used as probes to detect specific DNA segments. The sensitivity of this method is greatly enhanced when combined with PCR. Further, point mutations and other sequence variations, such as polymorphisms, can be detected as described above, for example, through the use of allele-specific oligonucleotides for PCR amplification of sequences that differ by single nucleotides.

DNA sequence differences may also be detected by alterations in the electrophoretic mobility of DNA fragments in gels, with or without denaturing agents, or by direct DNA sequencing (for example, Myers et al., Science (1985) 230:1242). Sequence changes at specific locations may also be revealed by nuclease protection assays, such as RNase and Si protection or the chemical cleavage method (see Cotton et al., Proc. Natl. Acad. Sci. USA (1985) 85: 4397-4401).

In addition to conventional gel electrophoresis and DNA sequencing, mutations such as microdeletions, aneuploidies, translocations, inversions, can also be detected by in situi analysis (see, for example, Keller et al., DNA Probes, 2nd Ed., Stockton Press, New York, N.Y., USA (1993)), that is, DNA or RNA sequences in cells can be analysed for mutations without need for their isolation and/or immobilisation onto a membrane. Fluorescence in situ hybridisation (FISH) is presently the most commonly applied method and numerous reviews of FISH have appeared (see, for example, Trachuck et al., Science, 250, 559-562 (1990), and Trask et al., Trends, Genet., 7, 149-154 (1991)).

In another embodiment of the invention, an array of oligonucleotide probes comprising a nucleic acid molecule according to the invention can be constructed to conduct efficient screening of genetic variants, mutations and polymorphisms. Array technology methods are well known and have general applicability and can be used to address a variety of questions in molecular genetics including gene expression, genetic linkage, and genetic variability (see for example: M. Chee et al., Science (1996), Vol 274, pp 610-613).

In one embodiment, the array is prepared and used according to the methods described in PCT application WO95/11995 (Chee et al); Lockhart, D. J. et al. (1996) Nat. Biotech. 14: 1675-1680); and Schena, M. et al. (1996) Proc. Natl. Acad. Sci. 93: 10614-10619). Oligonucleotide pairs may range from two to over one million. The oligomers are synthesized at designated areas on a substrate using a light-directed chemical process. The substrate may be paper, nylon or other type of membrane, filter, chip, glass slide or any other suitable solid support. In another aspect, an oligonucleotide may be synthesized on the surface of the substrate by using a chemical coupling procedure and an ink jet application apparatus, as described in PCT application WO95/251116 (Baldeschweiler et al). In another aspect, a “gridded” array analogous to a dot (or slot) blot may be used to arrange and link cDNA fragments or oligonucleotides to the surface of a substrate using a vacuum system, thermal, V, mechanical or chemical bonding procedures. An array, such as those described above, may be produced by hand or by using available devices (slot blot or dot blot apparatus), materials (any suitable solid support), and machines (including robotic instruments), and may contain 8, 24, 96, 384, 1536 or 6144 oligonucleotides, or any other number between two and over one million which lends itself to the efficient use of commercially-available instrumentation.

In addition to the methods discussed above, diseases may be diagnosed by methods comprising determining, from a sample derived from a subject, an abnormally decreased or increased level of polypeptide or mRNA. Decreased or increased expression can be measured at the RNA level using any of the methods well known in the art for the quantitation of polynucleotides, such as, for example, nucleic acid amplification, for instance PCR, RT-PCR, RNase protection, Northern blotting and other hybridisation methods.

Assay techniques that can be used to determine levels of a polypeptide of the present invention in a sample derived from a host are well-known to those of skill in the art and are discussed in some detail above (including radioimmunoassays, competitive-binding assays, Western Blot analysis and ELISA assays). This aspect of the invention provides a diagnostic method which comprises the steps of: (a) contacting a ligand as described above with a biological sample under conditions suitable for the formation of a ligand-polypeptide complex; and (b) detecting said complex.

Protocols such as ELISA, RIA, and FACS for measuring polypeptide levels may additionally provide a basis for diagnosing altered or abnormal levels of polypeptide expression. Normal or standard values for polypeptide expression are established by combining body fluids or cell extracts taken from normal mammalian subjects, preferably humans, with antibody to the polypeptide under conditions suitable for complex formation The amount of standard complex formation may be quantified by various methods, such as by photometric means.

Antibodies which specifically bind to a polypeptide of the invention may be used for the diagnosis of conditions or diseases characterised by expression of the polypeptide, or in assays to monitor patients being treated with the polypeptides, nucleic acid molecules, ligands and other compounds of the invention. Antibodies useful for diagnostic purposes may be prepared in the same manner as those described above for therapeutics. Diagnostic assays for the polypeptide include methods that utilise the antibody and a label to detect the polypeptide in human body fluids or extracts of cells or tissues. The antibodies may be used with or without modification, and may be labelled by joining them, either covalently or non-covalently, with a reporter molecule. A wide variety of reporter molecules known in the art may be used, several of which are described above.

Quantities of polypeptide expressed in subject, control and disease samples from biopsied tissues are compared with the standard values. Deviation between standard and subject values establishes the parameters for diagnosing disease. Diagnostic assays may be used to distinguish between absence, presence, and excess expression of polypeptide and to monitor regulation of polypeptide levels during therapeutic intervention. Such assays may also be used to evaluate the efficacy of a particular therapeutic treatment regimen in animal studies, in clinical trials or in monitoring the treatment of an individual patient.

A diagnostic kit of the present invention may comprise:

(a) a nucleic acid molecule of the present invention;

(b) a polypeptide of the present invention; or

(c) a ligand of the present invention.

In one aspect of the invention, a diagnostic kit may comprise a first container containing a nucleic acid probe that hybridises under stringent conditions with a nucleic acid molecule according to the invention; a second container containing primers useful for amplifying the nucleic acid molecule; and instructions for using the probe and primers for facilitating the diagnosis of disease. The kit may further comprise a third container holding an agent for digesting unhybridised RNA.

In an alternative aspect of the invention, a diagnostic kit may comprise an array of nucleic acid molecules, at least one of which may be a nucleic acid molecule according to the invention.

To detect polypeptide according to the invention, a diagnostic kit may comprise one or more antibodies that bind to a polypeptide according to the invention; and a reagent useful for the detection of a binding reaction between the antibody and the polypeptide.

Such kits will be of use in diagnosing a disease or disorder or susceptibility to disease or disorder in which midkines are implicated. Such diseases and disorders may include reproductive disorders, cell proliferative disorders, including neoplasm, melanoma, lung, colorectal, breast, pancreas, head and neck and other solid tumours; stomach cancer, colon cancer, pancreatic cancer, lung cancer, thoracic cancer, and liver cancer; myeloproliferative disorders, such as leukemia, non-Hodgkin lymphoma, leukopenia, thrombocytopenia, angiogenesis disorder, Kaposis' sarcoma; autoimmune/inflammatory disorders, including allergy, inflammatory bowel disease, pancreatitis, arthritis, psoriasis, psoriasis vulgaris, respiratory tract inflammation, asthma, and organ transplant rejection; cardiovascular disorders, including hypertension, oedema, angina, atherosclerosis, thrombosis, sepsis, shock, reperfusion injury, and ischemia, particularly ischemic heart disease; neurological disorders including central nervous system disease, Alzheimer's disease, brain injury, Parkinson's disease, amyotrophic lateral sclerosis, and pain; developmental disorders; metabolic disorders including diabetes mellitus, osteoporosis, and obesity, AIDS, renal disease, particularly idiopathic nephrotic syndrome; disorders related to fibrinolysis; neutrophilic functional disorders (e.g. lazy-leukocyte (chemotaxis-deficient leukocyte) syndrome); inflammatory diseases; wound healing disorders; lung injury; infections including viral infection, bacterial infection, fimgal infection and parasitic infection and other pathological conditions. Preferably, the disease is one in which midkines are implicated.

Various aspects and embodiments of the present invention will now be described in more detail by way of example, with particular reference to the INSP106 polypeptide.

It will be appreciated that modification of detail may be made without departing from the scope of the invention.

### BRIEF DESCRIPTION OF THE FIGURES

FIG. 1 Organisation of the known human midkine gene (P21741) and the splice variant INSP106.

FIG. 2 Multiple alignments of midldne family members including P21741 (first line) and INSP106 (second line, named sv1_chr11 in alignment), indicated with arrows.

FIG. 3 Dominant negative function of INSP106.

FIG. 4 Pairwise alignment of known midkine (P21741, named swall in alignment) and INSP106 (named chr11 in alignment).

FIG. 5 Nucleotide sequence of INSP106 prediction with translation.

FIG. 6 Nucleotide sequence with translation of INSP106 PCR product cloned using primers INSP106-CP1 and INSP106-CP2.

FIG. 7 Map of pCR4-TOPO-INSP106.

FIG. 8 Map of pDONR 221.

FIG. 9 Map of expression vector pEAK12d.

FIG. 10 Map of Expression vector pDEST12.2.

FIG. 11 Map of pDONR221-INSP106-6HIS.

FIG. 12 Map of pEAK12d-INSP106-6HIS.

FIG. 13 Map of pDEST12.2-INSP106-6HIS.

### EXAMPLES

### Example 1

Alternative pre-mRNA splicing is a major cellular process by which functionally diverse proteins can be generated from the primary transcript of a single gene, often in tissue specific patterns.

Experimentally, splice variants are identified by the fortuitous isolation and subsequent sequencing of variant mRNAs. However, this experimental approach has not been exhaustively completed for the human transcriptome (since this would require systematic isolation and sequencing of all mRNAs from all human tissues under all possible environmental conditions) and due to this experimental limitation there remains a large number of splice variants which have yet to be identified.

We have used proprietary bioinformatic approaches to perform a purposeful, directed search for the existence of splice variants of the human midline gene. By this method the limited data set of experimentally lnown splice variants can be extended to a much larger set of predicted splice variants.

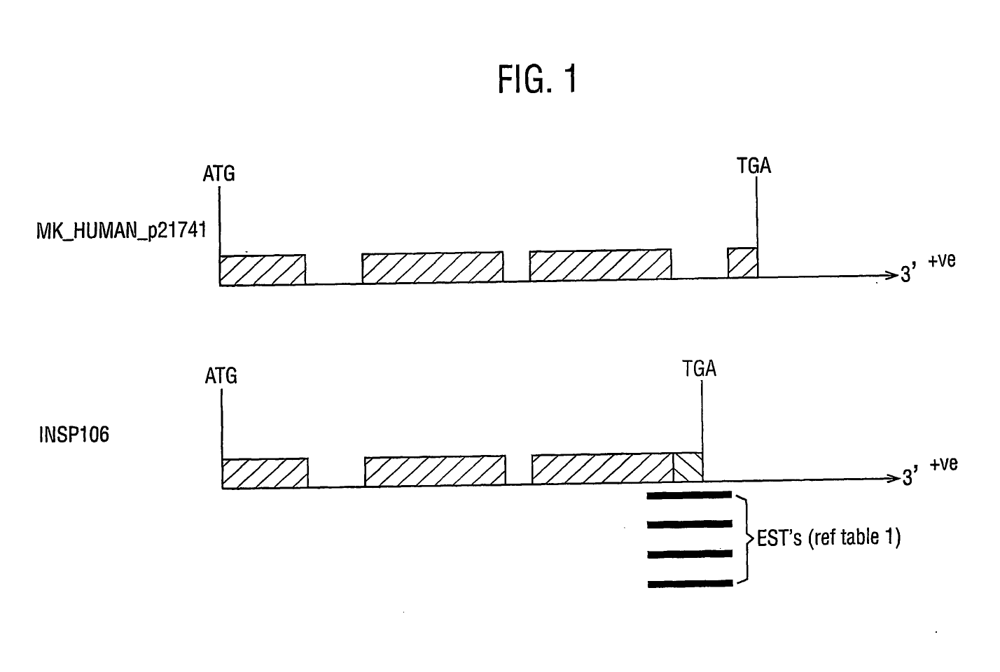

A gene model illustrating the variation within INSP106 in comparison to the known midkine shows that the 3^{rd} coding exon has been extended in the 3′ direction instead of having a separate 4^{th} coding exon present in P21741 (FIG. 1).

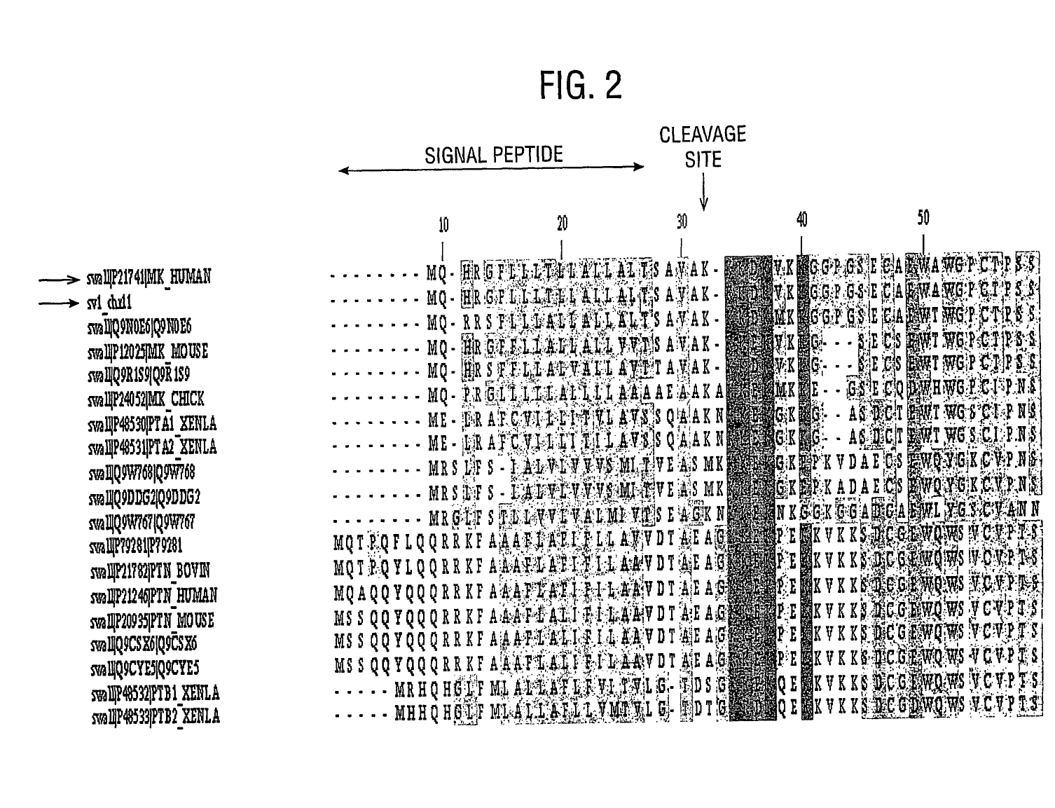

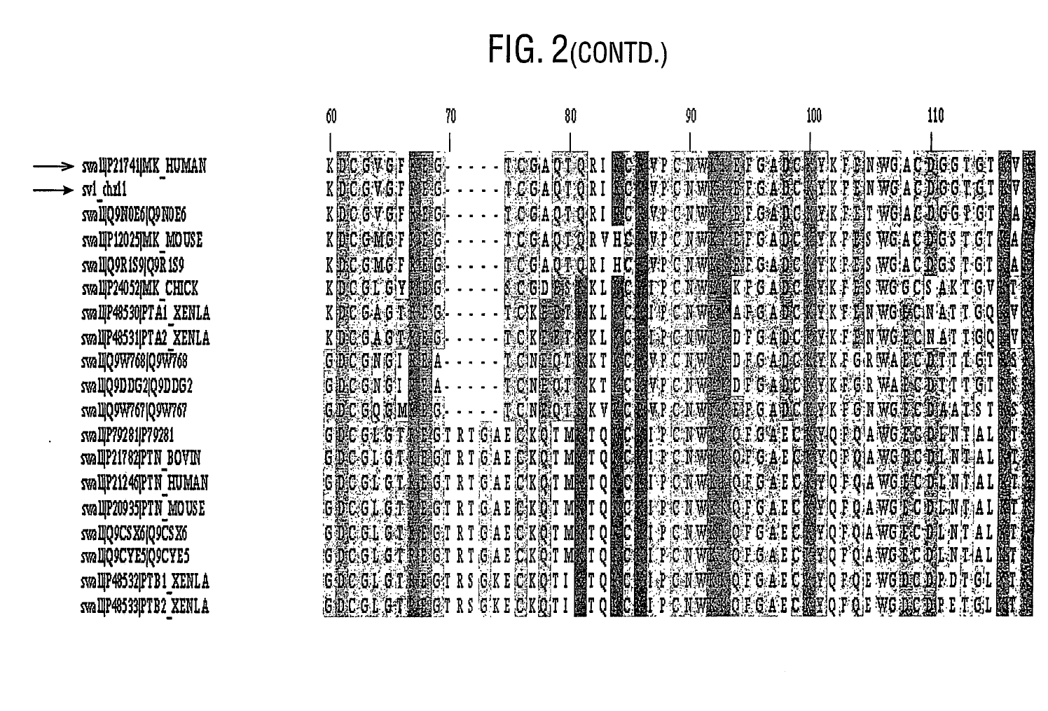

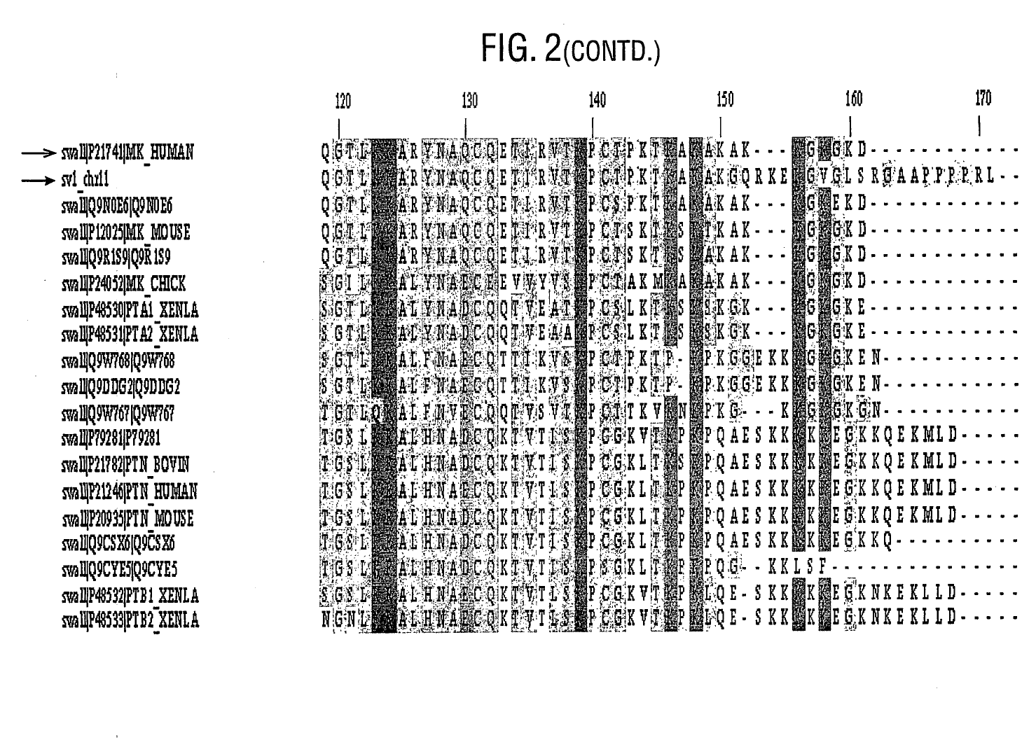

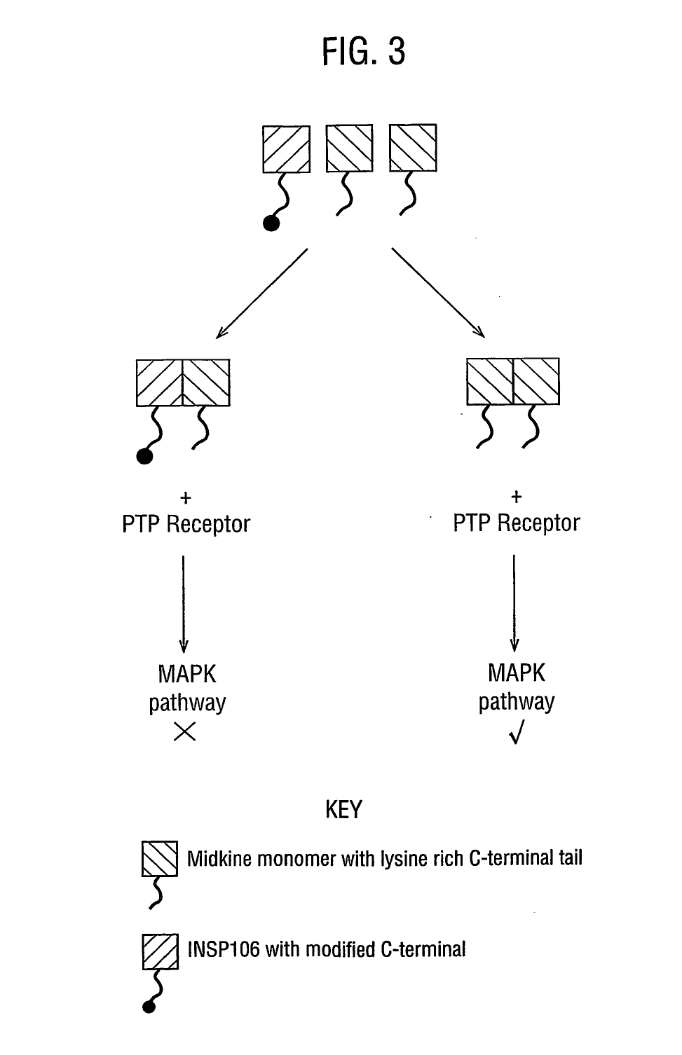

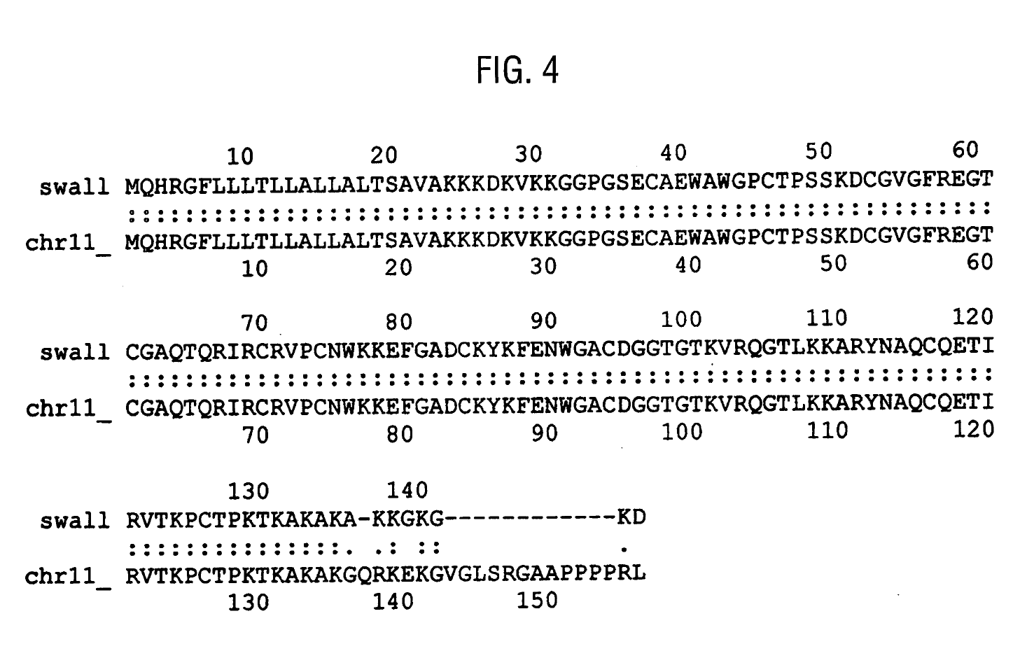

The multiple alignment demonstrates how a hydrophobic proline ‘tract’ extends the INSP106 prediction in the C terminal tail (FIG. 2).

### Example 2

### Neurobiology Assays Suitable for Exploration of the Biological Relevance of INSP106 Function

A number of neurobiology-related assays have been developed by the Applicant and are of use in the investigation of the biological relevance of INSP106 function and the identification of therapeutically useful moieties. Hence, in a preferred embodiment of the invention one or more of the following assays are used to identify a therapeutically useful moiety.

A. Oligodendrocyte Assays:

Oligodendrocytes are responsible for myelin formation in the CNS. In multiple sclerosis they are the first cells attacked and their loss leads to major behavioural impairment. In addition to curbing inflammation, enhancing the incomplete remyelination of lesions that occurs in MS has been proposed as a therapeutic strategy for MS. Like neurons, mature oligodendrocytes do not divide but the new oligodendrocytes can arise from progenitors. There are very few of these progenitor cells in adult brain and even in embryos the number of progenitor cells is inadequate for high-throughput screening. Therefore it is useful to look for oligodendrocyte cell lines that would fulfil the following criteria: high proliferative capacity, culture conditions compatible with high-throughput screening, and possibility to induce differentiation with proteins known to act in primary oligodendrocytes.

Oli-neu is a murine cell line obtained by an immortalization of an oligodendrocyte precursor by the t-neu oncogene. They are well studied and accepted as a representative cell line to study young oligodendrocyte biology (for example, see Schuster et al., J. Neurosci. Res. 2003 Aug. 1;73(3):324-33). Using this cell line two types of assays may be developed. The first type of assay can be used to identify factors that stimulate oligodendrocyte proliferation, and the other type can be used to identify factors promoting oligodendrocyte differentiation. Both events are key in the perspective of helping renewal and repairing demyelinating diseases.

The assays may also involve a human cell line, such as MO3-13. MO3-13 results from the fusion of rabdo-myosarcoma cells with adult human oligodendrocytes (see McLaurin et al., J Neurobiol. 1995 February;26(2):283-93). These cells have a reduced ability to differentiate into oligodendrocytes and their proliferating rate is not sufficient to allow a proliferation assay. Nevertheless, they express certain features of oligodendrocytes and their morphology is well adapted to nuclear translocation studies. The Applicant has developed assays based on nuclear translocation of three transcription factors, NF-KB, Stat-1 and Stat-2 in MO3-13 cells. The Jak/Stats transcription pathway is a complex pathway activated by many factors such as IFN α,β,?, cytokines (for example, IL-2, IL-6 and IL-5) or hormones (for example, GH, TPO, EPO). The specificity of the response depends on the combination of activated Stats. For example, it is noticeable that INF-β activates Statl, 2 and 3 nuclear translocations. In contrast, INF-? activates only Statl. In the same way, many cytokines and growth factors induce NF-kB translocation. In such assays the goal should be to get a picture of the pathways activated by the INSP106 protein. Thus, these assays provide a way of investigating whether the INSP106 polypeptide plays a role in the Jak/Stats transcription pathway. Complementary assays studying activation of other key pathways such as the PI3K, CREB and MEK pathways may be utilised to provide a full signalosome picture of INSP106.

B. Astrocyte Assays:

The biology of astrocytes is very complex but two general states are recognised. In the ‘quiescent’ state astrocytes regulate the metabolic and excitatory level of neurons by pumping glutamate and providing energetic substratum to neurons and oligodendrocytes. In the ‘activated’ state, astrocytes produce chemoliines and cytokines as well as nitric oxide. The first state can be considered as normal and healthy, while the second state is implicated in inflammation, stroke and neurodegenerative diseases. When this activated state persists it can be regarded as a pathological state.

Many factors and many pathways are known to modulate astrocyte activation. hn order to identify whether INSP106 modulates astrocyte activation, assays may employ U373 cells, a human cell line of astroglioma origin. NF-KB, c-Jun as well as Stats are signalling molecules known to play pivotal roles in astrocyte activation. The Applicant has therefore developed a series of screens based on the nuclear translocation of NF-kB, c-Jun and Stat1, 2 and 3. Prototypical activators of these pathways are IL-1b, IFN-beta or IFN-gamma. The goal in these assays is to identify whether the INSP106 proteins could be used as therapeutics themselves and to identify proteins and receptors that could be targeted for diagnostic or therapeutic applications.

C. Neuronal Assays:

Neurons are very complex and diverse cells but they have all in common two things. First they are post-mitotic cells, and secondly they are innervating other cells. Their survival is linked to the presence of trophic factors often produced by the irmervated target cells. In many neurodegenerative diseases, the loss of target innervation leads to cell body atrophy and apoptotic cell death. Therefore identification of trophic factors supplementing target deficiency is very important in treatment of neurodegenerative diseases. Accordingly, it is possible to set-up a survival assay using NS1 cells, a sub-clone of rat PC12 cells. These cells have been used for years and a lot of neurobiology knowledge has been first acquired on these cells before being confirmed on primary neurons including the pathways involved in neuron survival and differentiation (MEK, PI3K, CREB). In contrast the N2A cell line, a mouse neuroblastoma, does not respond to classical neurotrophic factors but Jun-kinase inhibitors prevent apoptosis induced by serum deprivation. Therefore, developing independent assays on these two cell lines will help to identify different types of survival-promoting proteins.

The above assays can be used to identify whether the INSP106 polypeptides promote both proliferation and differentiation or the relevant cell types. In order to identify whether the INSP106 polypeptides specifically promote neuronal differentiation, a NS1 differentiation assay based on neurite outgrowth has been developed. Promoting axonal or dendritic sprouting in neurodegenerative diseases could be advantageous for two reasons. It will first help the degenerating neurons to regrow and reestablish a contact with the target cells. Secondly, it will potentiate the so-called collateral sprouting from healthy fibers, a compensatory phenomenon that delays terminal phases of neurodegenerative diseases such as Parkinson or AD.

D. Endothelial Cell Assays:

The blood brain barrier (BBB) between brain and vessels is responsible for differences between cortical spinal fluid and serum compositions. The BBB results from a tight contact between endothelial cells and astrocytes. It maintains an immunotolerant status by preventing leukocytes penetration in brain, and allows the development of two parallel endocrine systems using the same intracellular signalling pathways. However, in many diseases or traumas, the BBB integrity is altered and leukocytes as well as serum proteins enter the brain inducing neuroinflammation. There is no simple in vitro model of BBB, but cultures of primary endothelial cells such as human embryonic umbilical vein endothelial cells (HUVEC) are considered to mimic some aspects of BBB biology. For example, BBB leakiness could be induced by proteins stimulating intracellular calcium release. In the perspective of identifying proteins that modulate BBB integrity, a calcium mobilization assay with or without thrombin has been developed by the Applicant using HUVEC.

### Example 3

### Cloning of INSP106

cDNA Libraries

Human cDNA libraries (in bacteriophage lambda (λ) vectors) were purchased from Stratagene or Clontech or prepared at the Serono Pharmaceutical Research Institute in λ ZAP, λ GT10, λ GT11, or TriplEx2 vectors according to the manufacturer's protocol (Stratagene and Clontech). Bacteriophage λ DNA was prepared from small scale cultures of infected E. coli host strain using the Wizard Lambda Preps DNA purification system according to the manufacturer's instructions (Promega, Corporation, Madison Wis.).

Preparation of Human cDNA Templates

First strand cDNA was prepared from a variety of normal human tissue total RNA samples (Clontech, Stratagene, Ambion, Biochain Institute and in-house preparations) using Superscript II RNase H⁻ Reverse Transcriptase (Invitrogen) according to the manufacturer's protocol. 1 μl Oligo (dT)₁₅ primer (500 μg/ml, Promega), 2 μg human total RNA, 1 μl 10 mM dNTP Mix (10 mM each dATP, dGTP, dCTP and dTTP at neutral pH) and sterile distilled water to a final volume of 12 μl were combined in a 1.5 ml Eppendorf tube, heated to 65° C. for 5 min and then chilled on ice. The contents were collected by brief centrifugation and 4 μl 5× First-Strand Buffer, 2 μl 0.1 M DTT, and 1 μl RnaseOUT Recombinant Ribonuclease Inhibitor (40 units/μl, Invitrogen) were added. The contents of the tube were mixed gently and incubated at 42° C. for 2 min, then 1 μl (200 units) of SuperScript II enzyme was added and mixed gently by pipeting. The mixture was incubated at 42° C. for 50 min and then inactivated by heating at 70° C. for 15 min. To remove RNA complementary to the cDNA, 1 μl (2 units) of E. coli RNase H (Invitrogen) was added and the reaction mixture incubated at 37° C. for 20 min. The final 21 μl reaction mix was diluted by adding 179 μl sterile water to give a total volume of 200 μl. First strand cDNA samples were combined into pools of 5 samples, each pool containing 1 μl of each cDNA template. 5 μl of each pool was used as the template in amplification reactions.

Gene Specific Cloning Primers for PCR

A pair of PCR primers having a length of between 18 and 25 bases were designed for amplifying the complete coding sequence of the virtual cDNA using Primer Designer Software (Scientific & Educational Software, PO Box 72045, Durham, N.C. 27722-2045, USA). PCR primers were optimized to have a Tm close to 55±10° C. and a GC content of 40-60%. Primers were selected which had high selectivity for the target sequence (INSP106) with little or no none specific priming.

PCR Amplification of INSP106 from a Variety of Human cDNA Templates

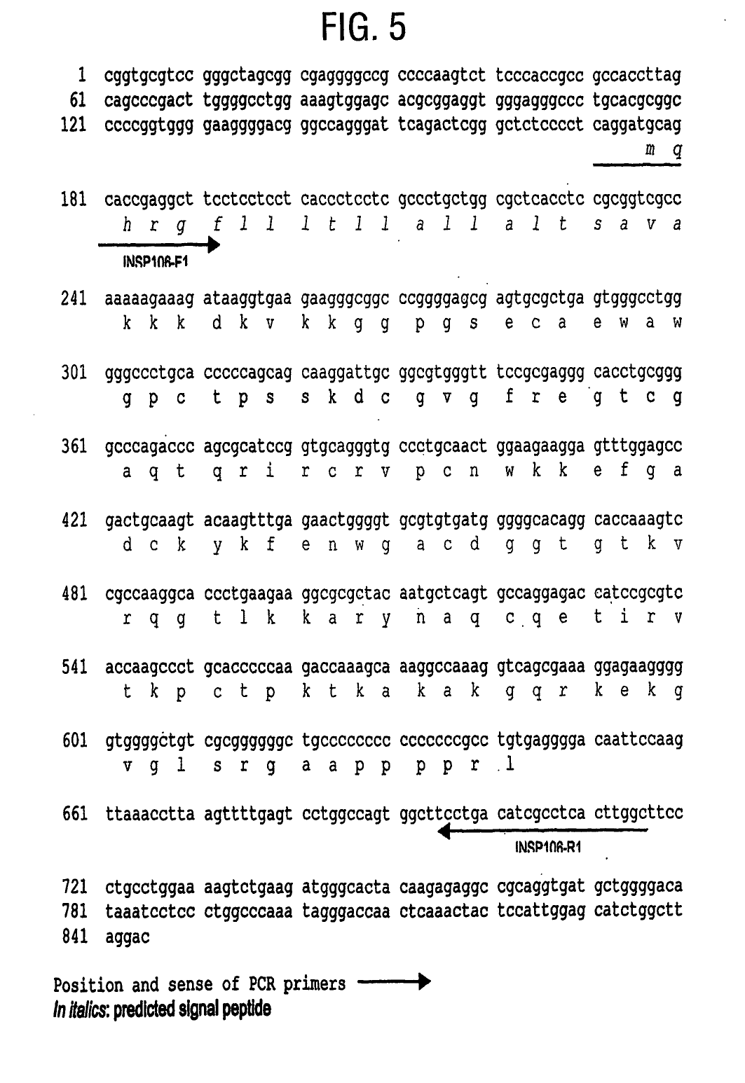

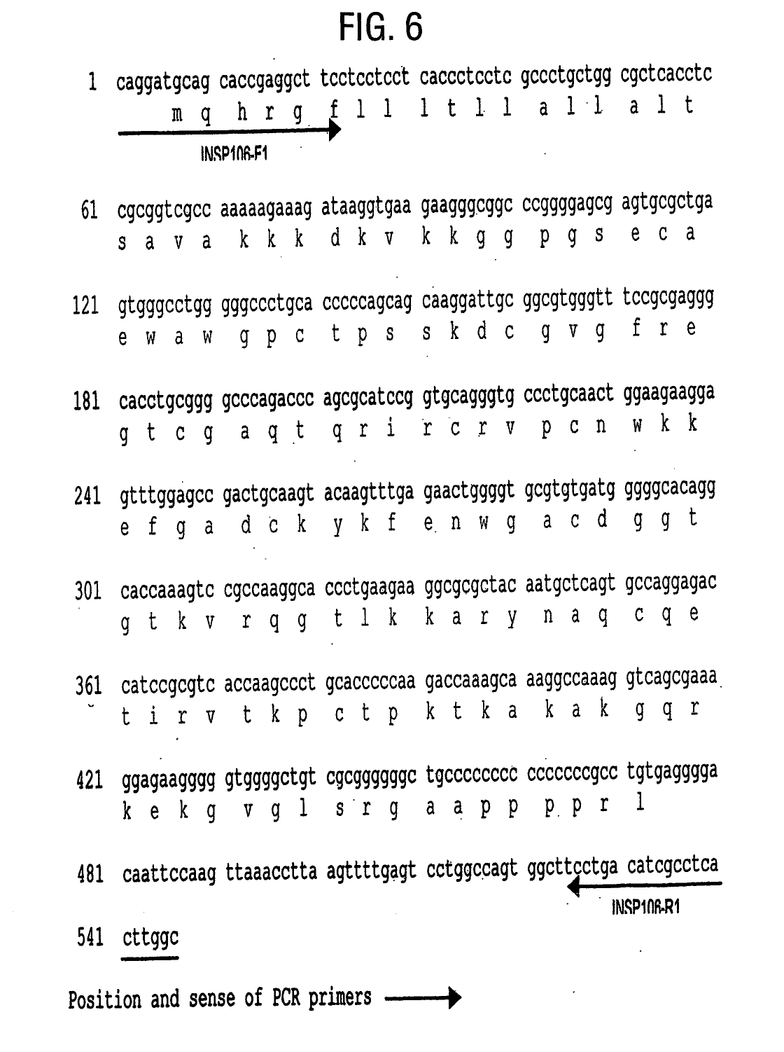

Gene-specific cloning primers (INSP106-CP1 and INSP106-CP2, FIG. 5, FIG. 6 and Table 2) were designed to amplify a cDNA fragment of 546 bp covering the entire 471 bp coding sequence of the INSP106 prediction. The primer pair was used with a range of λ cDNA library samples and pools of human cDNA samples as PCR templates. The PCR was performed in a final volume of 50 μl containing 1× AmpliTaq™ buffer, 200 μM dNTPs, 50 pmoles each of cloning primer, 2.5 units of AmpliTaq™ (Perlin Elmer) and 100 ng of each λ cDNA library template or 5 μl of each cDNA pool using an MJ Research DNA Engine, programmed as follows: 94° C., 2 min; 40 cycles of 94° C., 1 min, 65° C., 1 min, and 72° C., 1 min; followed by 1 cycle at 72° C. for 7 min and a holding cycle at 4° C. The amplification products were visualized on 0.8 % agarose gels in 1× TAE buffer (Invitrogen). PCR products migrating at the predicted molecular mass were purified from the gel using the Wizard PCR Preps DNA Purification System (Promega). The PCR product was eluted in 50 μl of sterile water and either subcloned directly or stored at −20° C.

Subcloning of PCR Products

The PCR products were subcloned into the topoisomerase I modified cloning vector (pCR4-TOPO) using the TA cloning kit purchased from the Invitrogen Corporation using the conditions specified by the manufacturer. Briefly, 4 μl of gel purified PCR product was incubated for 15 min at room temperature with 1 μl of TOPO vector and 1 μl salt solution. The reaction mixture was then transformed into E. coli strain TOP 10 (Invitrogen) as follows: a 50 μl aliquot of One Shot TOP10 cells was thawed on ice and 2 μl of TOPO reaction was added. The mixture was incubated for 15 min on ice and then heat shocked by incubation at 42° C. for exactly 30 s. Samples were returned to ice and 250 μl of warm (room temperature) SOC media was added. Samples were incubated with shaking (220 rpm) for 1 h at 37° C. The transformation mixture was then plated on L-broth (LB) plates containing ampicillin (100 μg/ml) and incubated overnight at 37° C.

Colony PCR

Colonies were inoculated into 50 μl sterile water using a sterile toothpick. A 10 μl aliquot of the inoculum was then subjected to PCR in a total reaction volume of 20 μl containing 1× AmpliTaq™ buffer, 200 μM dNTPs, 20 pmoles of T7 primer, 20 pmoles of T3 primer, 1 unit of AmpliTaq™ (Perkin Elmer) using an MT Research DNA Engine. The cycling conditions were as follows: 94° C., 2 min; 30 cycles of 94° C., 30 sec, 48° C., 30 sec and 72° C. for 1 min. Samples were maintained at 4° C. (holding cycle) before further analysis.

PCR reaction products were analyzed on 1 % agarose gels in 1× TAE buffer. Colonies which gave the expected PCR product size (546 bp cDNA+105 bp due to the multiple cloning site or MCS) were grown up overnight at 37° C. in 5 ml L-Broth (LB) containing ampicillin (100 μg/ml), with shaking at 220 rpm.

Plasmid DNA Preparation and Sequencing

Miniprep plasmid DNA was prepared from 5 ml cultures using a Qiaprep Turbo 9600 robotic system (Qiagen) or Wizard Plus SV Minipreps kit (Promega cat. no. 1460) according to the manufacturer's instructions. Plasmid DNA was eluted in 100 μl of sterile water. The DNA concentration was measured using an Eppendorf BO photometer. Plasmid DNA (200-500 ng) was subjected to DNA sequencing with the T7, T3, INSP106-SP1, and INSP106-SP2 primers using the BigDyeTerninator system (Applied Biosystems cat. no. 4390246) according to the manufacturer's instructions. The primer sequences are shown in Table 2. Sequencing reactions were purified using Dye-Ex columns (Qiagen) or Montage SEQ 96 cleanup plates (Millipore cat. no. LSKS09624) then analyzed on an Applied Biosystems 3700 sequencer.

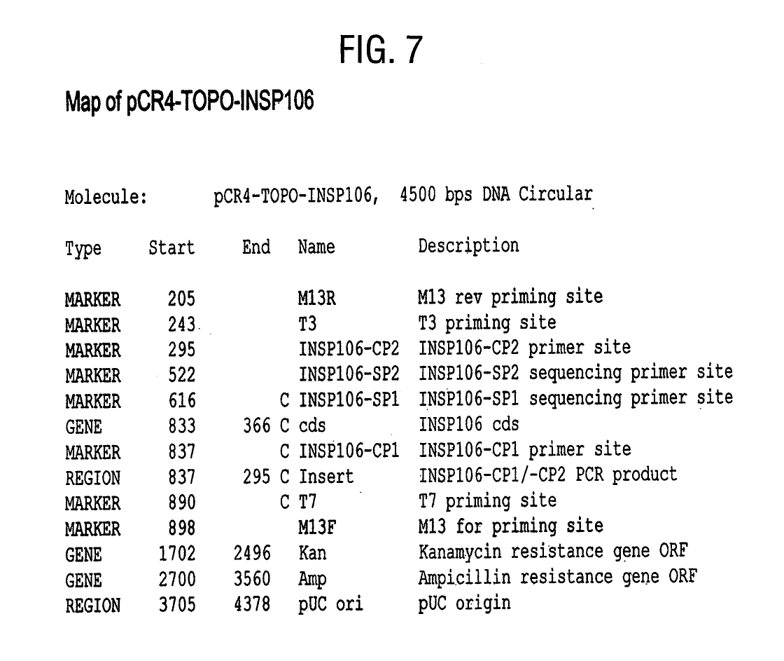

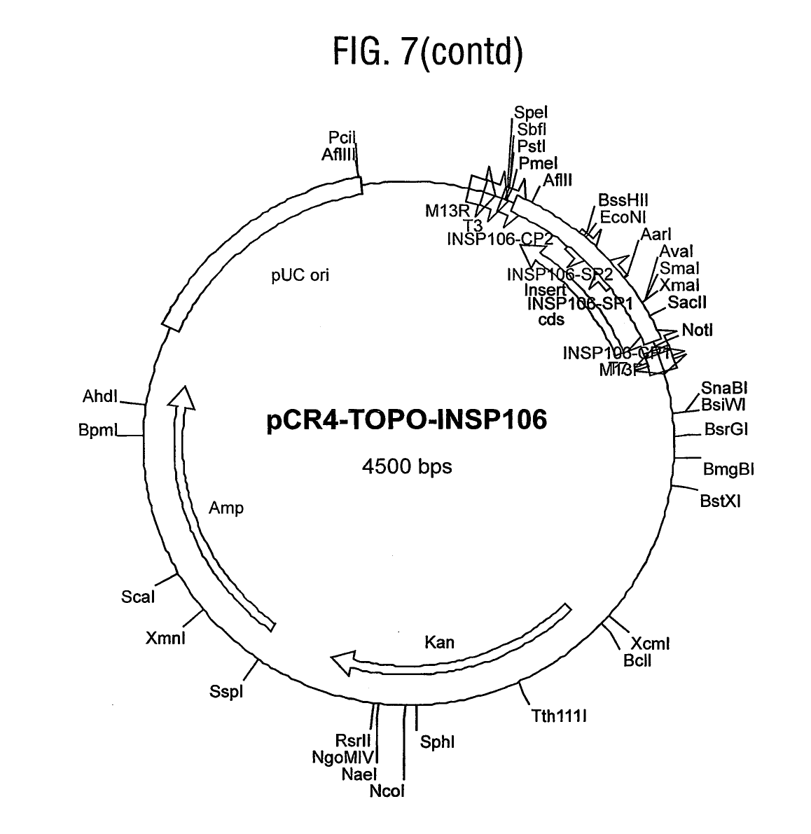

Sequence analysis identified a clone containing 100% match to the predicted INSP106 ORF sequence. This sequence had been amplified from a pool of cDNA templates derived from human SK-N-MC, SK-N-AS, TE671, KELLY and U-373 cell lines. The sequence of the cloned cDNA fragment is shown in FIG. 6. The plasmid map of the cloned PCR product (pCR4-TOPO-INSP106) (plasmid ID.13864) is shown in FIG. 7.

Construction of a Plasmid for the Expression of INSP106 in HEK293/EBNA Cells.

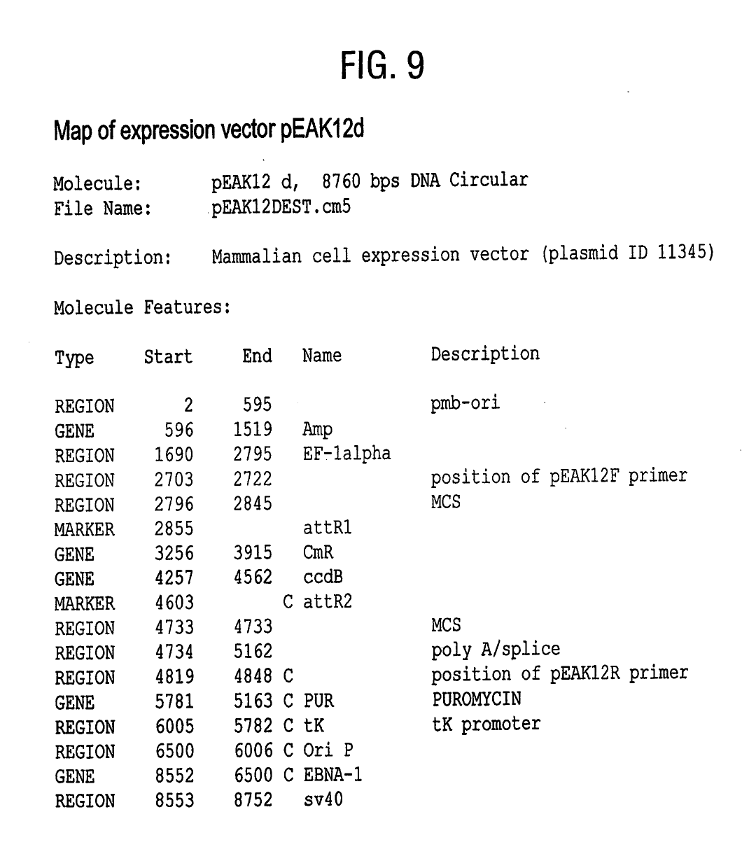

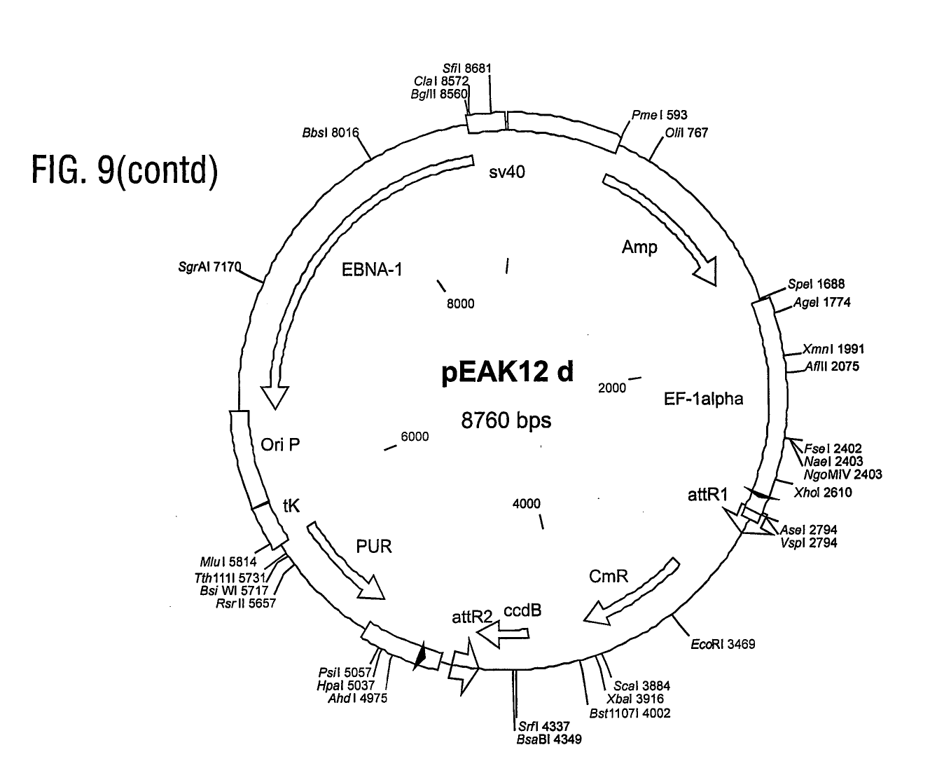

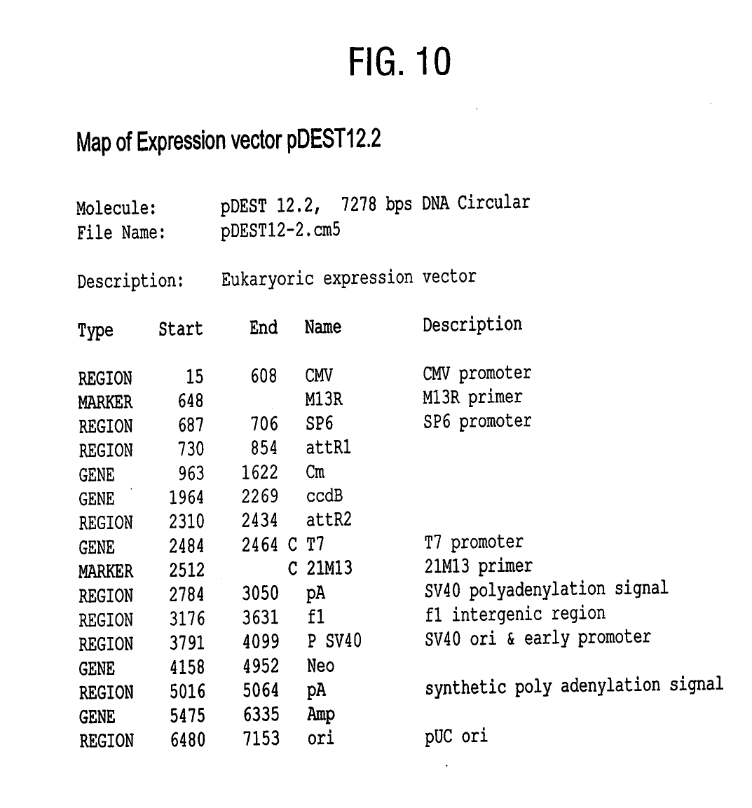

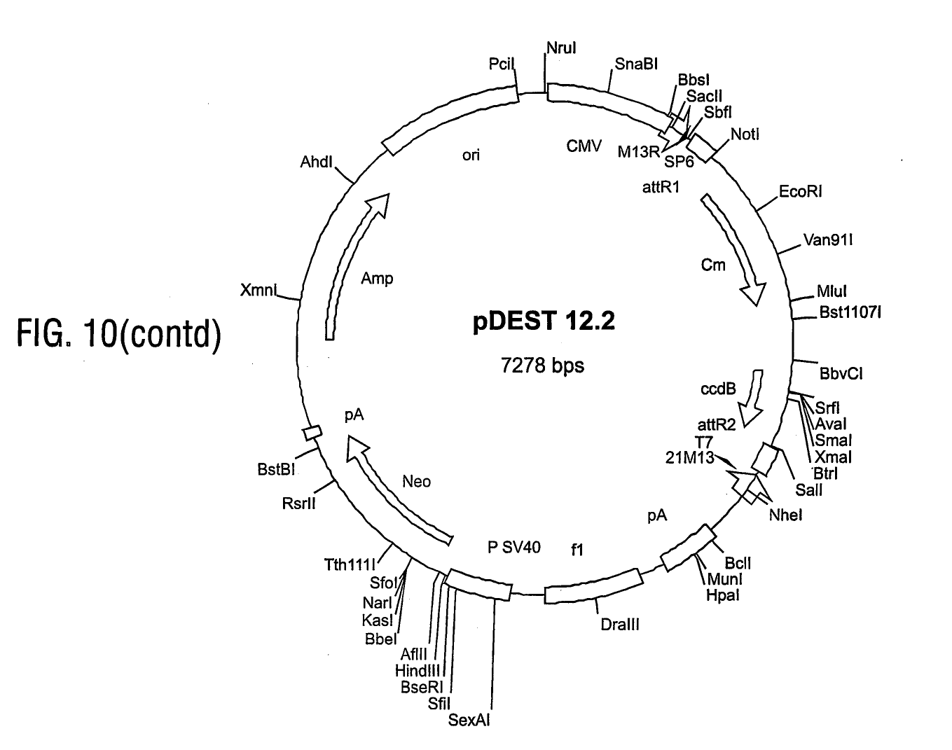

A pCR4-TOPO clone containing the full coding sequence (ORF) of INSP106 identified by DNA sequencing (pCR4-TOPO-INSP106, plasmid ID.13864) (FIG. 7) was then used to subclone the insert into the mammalian cell expression vectors pEAK12d (FIG. 9) and pDEST12.2 (FIG. 10) using the Gateway™ cloning methodology (Invitrogen).

Generation of Gateway Compatible INSP106 ORF Fused to an In Frame 6HIS Tag Sequence.

The first stage of the Gateway cloning process involves a two step PCR reaction which generates the ORF of INSP106 flanked at the 5′ end by an attBl recombination site and Kozak sequence, and flanked at the 3′ end by a sequence encoding an in frame 6 histidine (6HIS) tag, a stop codon and the attB2 recombination site (Gateway compatible cDNA). The predicted INSP106 sequence contained a run of 15 C bases at its 3′ end, predominantly encoding a run of 4 proline residues. This would make amplification and sequencing of this region very difficult, and so to remedy this problem the codon usage of this region was altered when the reverse amplification primer INSP106-EX2 was designed (Table 2).

The first PCR reaction (in a final volume of 50 μl) contains: 1.5 μl of pCR4-TOPO-INSP106 (plasmid ID 13864), 1.5 μl dNTPs (10 mM), 5 μl of 10× Pfx polymerase buffer, 1 μl MgSO₄ (50 mM), 0.5 μl each of gene specific primer (100 μM) (INSP106-EX1 and INSP106-EX2), 2.5 μl 10× Enhancers solution (Invitrogen) and 1 μl Platinum Pfx DNA polymerase (Invitrogen). The PCR reaction was performed using an initial denaturing step of 95° C. for 2 min, followed by 15 cycles of 94° C. for 15 s; 55° C. for 30 s and 68° C. for 2 min 30 sec; and a holding cycle of 4° C. Reaction products were analysed on a 1% agarose gel (1× TAE). PCR products of the correct size (468 bp) were gel purified using the Qiagen MinElute DNA purification system (Qiagen) according to the manufacturer's instructions, and eluted in 10 μl of EB buffer (10 mM Tris.Cl, pH 8.5).

The second PCR reaction (in a final volume of 50 μl) contained 8 μl purified PCR 1 product, 1.5 μl dNTPs (10 mM), 5 μl of 10× Pfx polymerase buffer, 1 μl MgSO₄ (50 mM), 0.5 μl of each Gateway conversion primer (100 μM) (GCP forward and GCP reverse) and 0.5 μl of Platinum Pfx DNA polymerase. The conditions for the 2nd PCR reaction were: 95° C. for 1 min; 4 cycles of 94° C., 15 sec; 50° C., 30 sec and 68° C. for 2 min 30 sec; 19 cycles of 94° C., 15 sec; 55° C., 30 sec and 68° C., 2 min 30 sec; followed by a holding cycle of 4° C. PCR products were gel purified using the Wizard PCR prep DNA purification system (Promega) according to the manufacturer's instructions.

Subcloning of Gateway Compatible INSP106 ORF Into Gateway Entry Vector pDONR221 and Expression Vectors pEAK12d and pDEST12.2

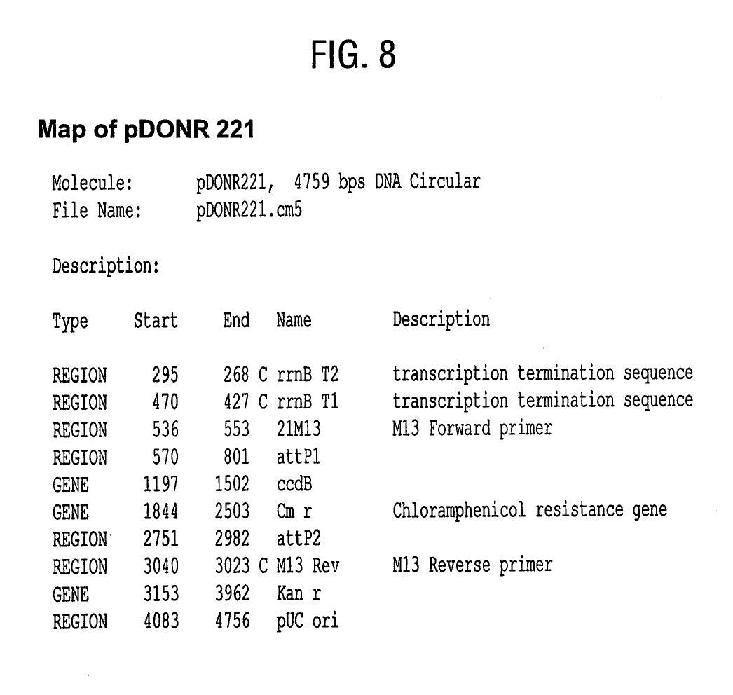

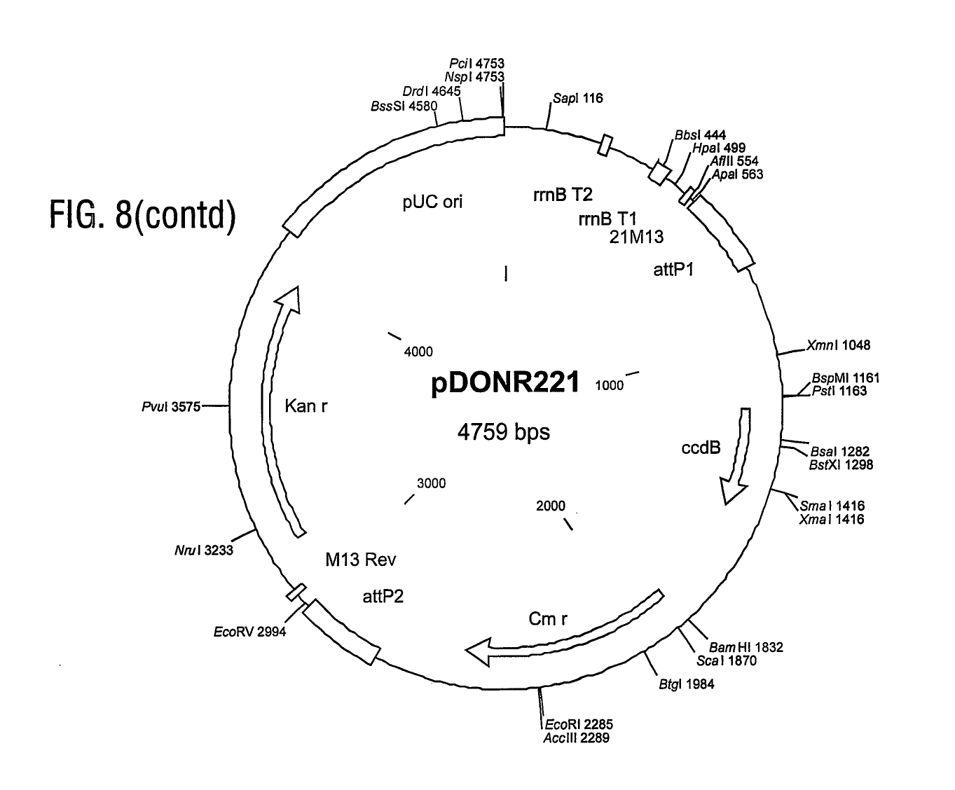

The second stage of the Gateway cloning process involves subcloning of the Gateway modified PCR product into the Gateway entry vector pDONR221 (Invitrogen, FIG. 8) as follows: 5 μl of purified product from PCR2 were incubated with 1 μl pDONR221 vector (0.15 μg/μl), 2 μl BP buffer and 1.5 μl of BP clonase enzyme mix (Invitrogen) in a final volume of 10 μl at RT for 1 h. The reaction was stopped by addition of proteinase K (2 pg) and incubated at 37° C. for a further 10 min. An aliquot of this reaction (2 μl) was used to transform E. coli DH10B cells by electroporation as follows: a 30 μl aliquot of DH10B electrocompetent cells (Invitrogen) was thawed on ice and 2 μl of the BP reaction mix was added. The mixture was transferred to a chilled 0.1 cm electroporation cuvette and the cells electroporated using a BioRad Gene-Pulser™ according to the manufacturer's recommended protocol. SOC media (0.5 ml) which had been pre-warmed to room temperature was added immediately after electroporation. The mixture was transferred to a 15 ml snap-cap tube and incubated, with shaking (220 rpm) for 1 h at 37° C. Aliquots of the transformation mixture (10 μl and 50 μl) were then plated on L-broth (LB) plates containing kanamycin (40 μg/ml) and incubated overnight at 37° C.

Plasmid mini-prep DNA was prepared from 5 ml cultures of a number of the resultant colonies using a Qiaprep Turbo 9600 robotic system (Qiagen). Plasmid DNA (200-500 ng) was subjected to DNA sequencing with 21M13, M13Rev, INSP106-SP1, and INSP106-SP2 primers using the BigDyeTerminator system (Applied Biosystems cat. no. 4390246) according to the manufacturer's instructions. The primer sequences are shown in Table 2. Sequencing reactions were purified using Dye-Ex columns (Qiagen) or Montage SEQ 96 cleanup plates (Millipore cat. no. LSKS09624) then analyzed on an Applied Biosystems 3700 sequencer.

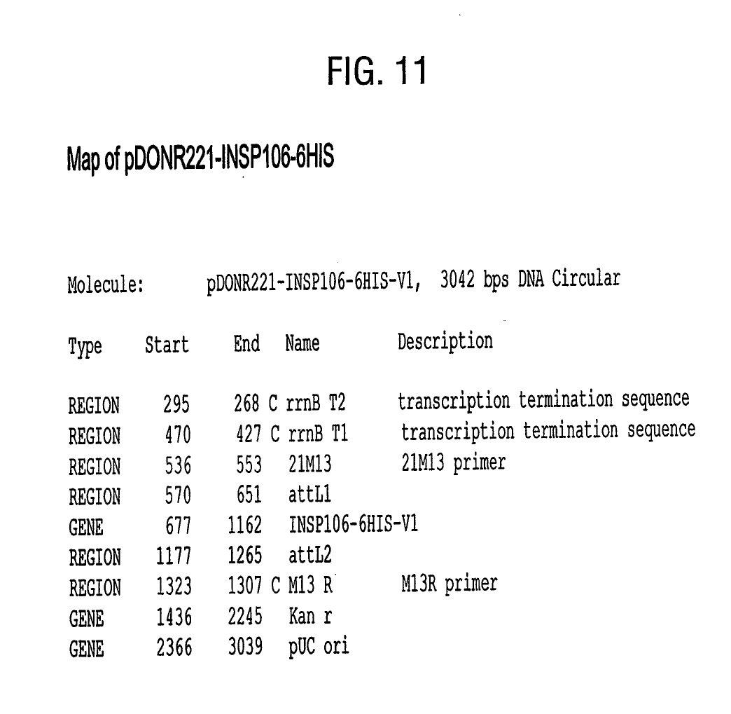

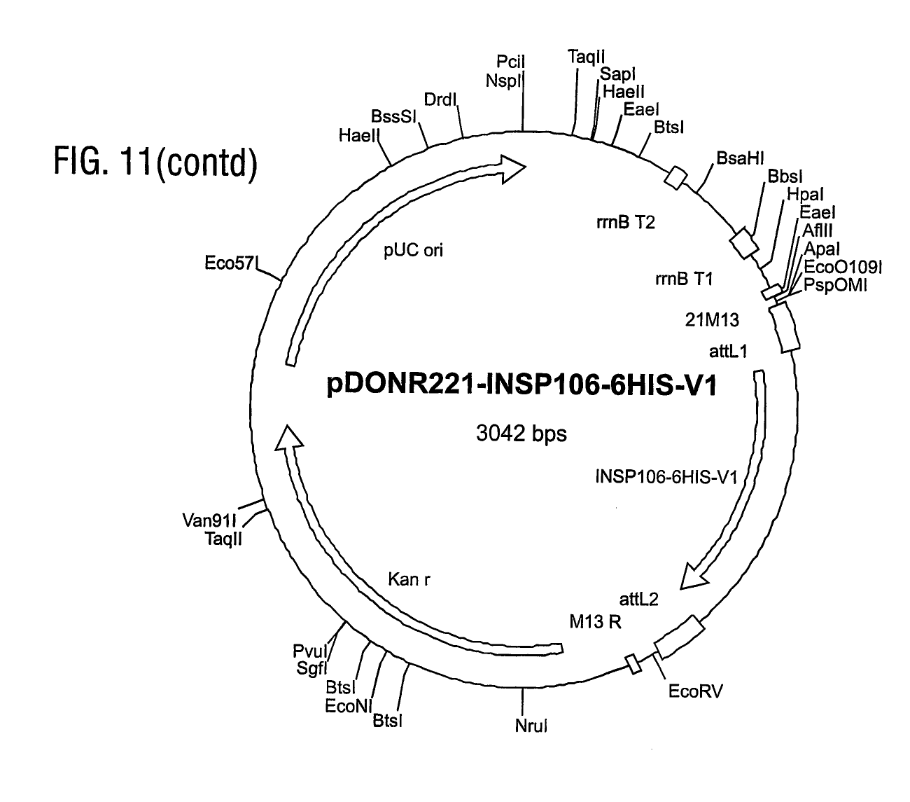

Plasmid eluate (2 μl) from one of the clones which contained the correct sequence (pENTR-INSP106-6HIS, plasmid ID 14342, FIG. 11) was then used in a recombination reaction containing 1.5 μl of either pEAK12d vector or pDEST12.2 vector (FIGS. 9 & 10) (0.1 μg/μl), 2 □l LR buffer and 1.5 μl of LR clonase (Invitrogen) in a final volume of 10 μl. The mixture was incubated at RT for 1 h, stopped by addition of proteinase K (2 μg) and incubated at 37° C. for a further 10 min. An aliquot of this reaction (1 ul) was used to transform E. coli DH10B cells by electroporation as follows: a 30 μl aliquot of DH10B electrocompetent cells (Invitrogen) was thawed on ice and 1 μl of the LR reaction mix was added. The mixture was transferred to a chilled 0.1 cm electroporation cuvette and the cells electroporated using a BioRad Gene-Pulser™ according to the manufacturer's recommended protocol. SOC media (0.5 ml) which had been pre-warmed to room temperature was added immediately after electroporation. The mixture was transferred to a 15 ml snap-cap tube and incubated, with shaking (220 rpm) for 1 h at 37° C. Aliquots of the transformation mixture (10 μl and 50 μl) were then plated on L-broth (LB) plates containing ampicillin (100 μg/ml) and incubated overnight at 37° C.

Plasmid mini-prep DNA was prepared from 5 ml cultures from a number of the resultant colonies subcloned in each vector using a Qiaprep Turbo 9600 robotic system (Qiagen). Plasmid DNA (200-500 ng) in the pEAK12d vector was subjected to DNA sequencing with pEAK12F, pEAK12R, INSP106-SP1, and INSP106-SP2 primers as described above. Plasmid DNA (200-500 ng) in the pDEST12.2 vector was subjected to DNA sequencing with 21M13, M13Rev, INSP106-SP1, and INSP106-SP2 primers as described above. Primers sequences are shown in Table 2.

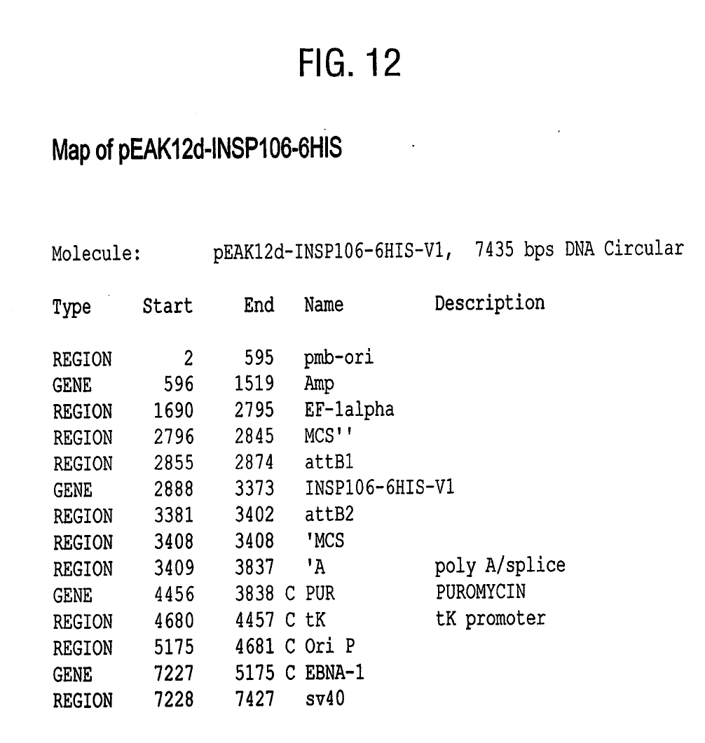

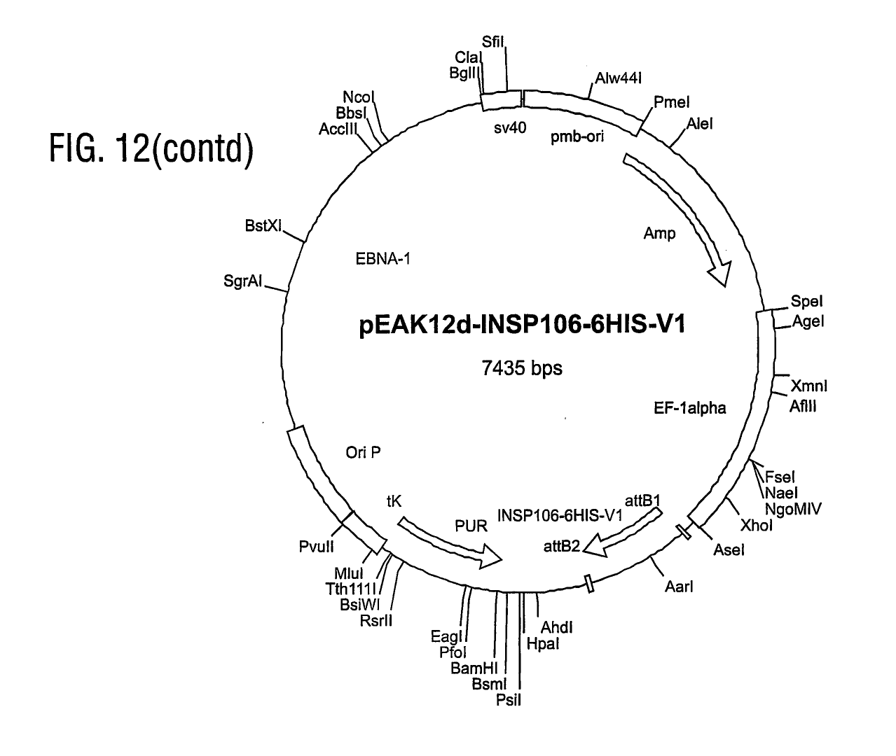

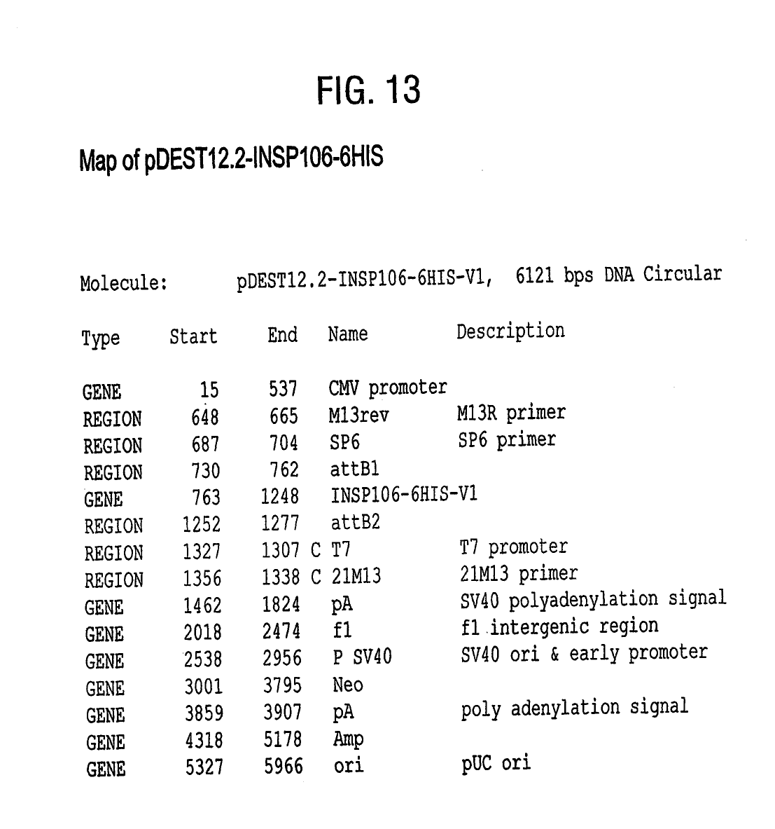

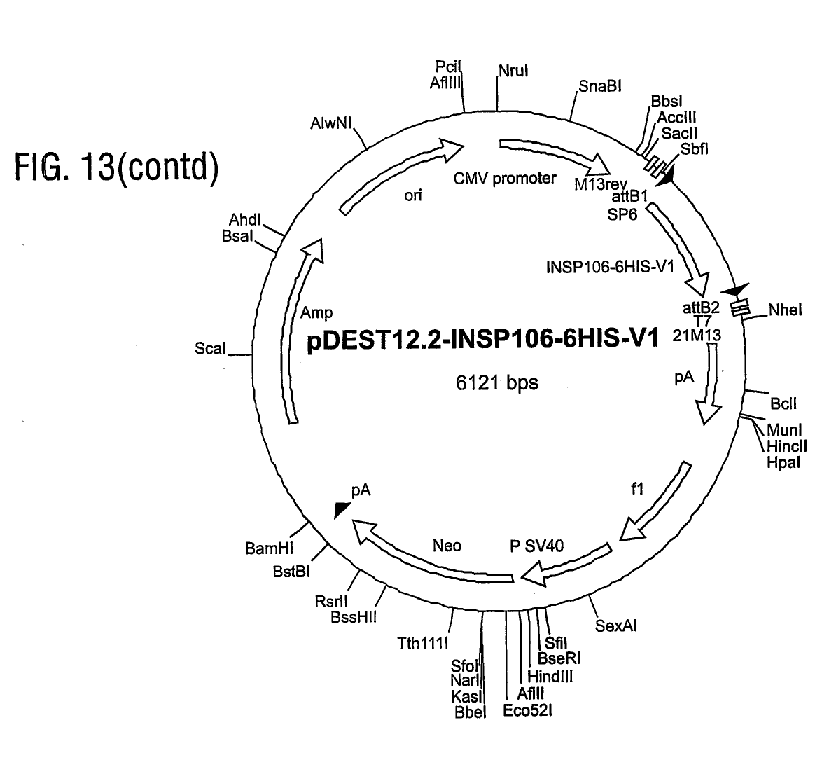

CsCl gradient purified maxi-prep DNA was prepared from a 500 ml culture of one of each of the sequence verified clones (pEAK12d-INSP106-6HIS, plasmid ID number 14344, FIG. 12, and pDEST12.2-INSP106-6HIS, plasmid ID 14421, FIG. 9) using the method described by Sambrook J. et al., 1989 (in Molecular Cloning, a Laboratory Manual, 2^{nd} edition, Cold Spring Harbor Laboratory Press). Plasmid DNA was resuspended at a concentration of 1 μg/μl in sterile water and stored at −20° C.

TABLE 2

Primers for INSP106 cloning and sequencing

| | |
|---|---|
| Primer | Sequence (5′-3′) |

| | |
|---|---|
| INSP106- | CAG GAT GCA GCA CCG AGG CTT C |
| CP1 | |
| INSP106- | GCC AAG TGA GGC GAT GTC AGG A |
| CP2 | |
| INSP106- | CCT GCA ACT GGA AGA AGG A |
| SP1 | |
| TNSP106- | TTG GCG GAC TTT GGT GCC TG |
| SP2 | |
| INSP106- | GCA GGC TTC GCC ACC ATG CAG CAC CGA GGC |
| EX1 | TTC CT |
| INSP106- | GTG ATG GTG ATG GTG CAG GCG TGG AGG TGG |
| EX2 | GGG GG |
| GCP | G GGG ACA AGT TTG TAC AAA AAA GCA GGC TTC |
| Forward | GCC ACC |
| GCP | GGG GAC CAC TTT GTA CAA GAA AGC TGG GTT |
| Reverse | TCA ATG GTG ATG GTG ATG GTG |
| pEAK12-F | GCC AGC TTG GCA CTT GAT GT |
| pEAK12-R | GAT GGA GGT GGA CGT GTC AG |
| pENTR-F | TCG CGT TAA CGC TAG CAT GGA TCT C |
| pENTR-R | GTA ACA TCA GAG ATT TTG AGA CAC |
| T7 | TAA TAC GAC TCA CTA TAG GG |
| T3 | CTC CCT TTA GTG AGG GTA ATT |

Underlined sequence = Kozak sequence
Bold = Stop codon
Italic sequence = His tag

### Example 4

### Expression and Purification of INSP106

Further experiments may now be performed to determine the tissue distribution and expression levels of the INSP106 polypeptides in vivo, on the basis of the nucleotide and amino acid sequence disclosed herein.

The presence of the transcripts for INSP106 may be investigated by PCR of cDNA from different human tissues. The INSP106 transcripts may be present at very low levels in the samples tested. Therefore, extreme care is needed in the design of experiments to establish the presence of a transcript in various human tissues as a small amount of genomic contamination in the RNA preparation will provide a false positive result. Thus, all RNA should be treated with DNAse prior to use for reverse transcription. In addition, for each tissue a control reaction may be set up in which reverse transcription was not undertaken (a −ve RT control).

For example, 1 μg of total RNA from each tissue may be used to generate cDNA using Multiscript reverse transcriptase (ABI) and random hexamer primers. For each tissue, a control reaction is set up in which all the constituents are added except the reverse transcriptase (−ve RT control). PCR reactions are set up for each tissue on the reverse transcribed RNA samples and the minus RT controls. INSP106-specific primers may readily be designed on the basis of the sequence information provided herein. The presence of a product of the correct molecular weight in the reverse transcribed sample together with the absence of a product in the minus RT control may be taken as evidence for the presence of a transcript in that tissue. Any suitable cDNA libraries may be used to screen for the INSP106 transcripts, not only those generated as described above.

The tissue distribution pattern of the INSP106 polypeptides will provide further useful information in relation to the function of those polypeptides.

In addition, further experiments may now be performed using the expression vectors disclosed herein. Transfection of mammalian cell lines with these vectors may enable the high level expression of the INSP106 polypeptides and thus enable the continued investigation of the functional characteristics of the INSP106 polypeptides. The following material and methods are an example of those suitable in such experiments:

Cell Culture

Human Embryonic Kidney 293 cells expressing the Epstein-Barr virus Nuclear Antigen (HEK293-EBNA, Invitrogen) are maintained in suspension in Ex-cell VPRO serum-free medium (seed stock, maintenance medium, JRH). 16 to 20 hours prior to transfection (Day-1), cells are seeded in 2× T225 flasks (50ml per flask in DMEM/F12 (1:1) containing 2% FBS seeding medium (JRH) at a density of 2×10⁵ cells/ml). The next day (transfection day 0) transfection takes place using the JetPEITM reagent (2 μl/μg of plasmid DNA, PolyPlus-transfection). For each flask, plasmid DNA is co-transfected with GFP (fluorescent reporter gene) DNA. The transfection mix is then added to the 2× T225 flasks and incubated at 37° C. (5% CO₂) for 6 days. Confirmation of positive transfection may be carried out by qualitative fluorescence examination at day 1 and day 6 (Axiovert 10 Zeiss).

On day 6 (harvest day), supernatants from the two flasks are pooled and centrifuged (e.g. 4° C., 400 g) and placed into a pot bearing a unique identifier. One aliquot (500 μl) is kept for QC of the 6His-tagged protein (internal bioprocessing QC).

Scale-up batches may be produced by following the protocol called “PEI transfection of suspension cells”, referenced BP/PEI/HH/02/04, with PolyEthylenelmine from Polysciences as transfection agent.

Purification Process

The culture medium sample containing the recombinant protein with a C-terminal 6His tag is diluted with cold buffer A (50 mM NaH₂PO₄; 600 mM NaCl; 8.7% (w/v) glycerol, pH 7.5). The sample is filtered then through a sterile filter (Millipore) and kept at 4° C. in a sterile square media bottle (Nalgene).

The purification is performed at 4° C. on the VISION workstation (Applied Biosystems) connected to an automatic sample loader (Labomatic). The purification procedure is composed of two sequential steps, metal affinity chromatography on a Poros 20 MC (Applied Biosystems) column charged with Ni ions (4.6×50 mm, 0.83 ml), followed by gel filtration on a Sephadex G-25 medium (Amersham Pharmacia) column (1.0×10 cm).

For the first chromatography step the metal affinity column is regenerated with 30 column volumes of EDTA solution (100 mM EDTA; 1M NaCl; pH 8.0), recharged with Ni ions through washing with 15 column volumes of a 100 mM NiSO₄ solution, washed with 10 column volumes of buffer A, followed by 7 column volumes of buffer B (50 mM NaH₂PO₄; 600 mM NaCl; 8.7% (w/v) glycerol, 400 mM; imidazole, pH 7.5), and finally equilibrated with 15 column volumes of buffer A containing 15 mM imidazole. The sample is transferred, by the Labomatic sample loader, into a 200 ml sample loop and subsequently charged onto the Ni metal affinity column at a flow rate of 10 ml/min. The column is washed with 12 column volumes of buffer A, followed by 28 column volumes of buffer A containing 20 mM imidazole. During the 20 mM imidazole wash loosely attached contaminating proteins are eluted from the column. The recombinant His-tagged protein is finally eluted with 10 column volumes of buffer B at a flow rate of 2 ml/min, and the eluted protein is collected.

For the second chromatography step, the Sephadex G-25 gel-filtration column is regenerated with 2 ml of buffer D (1.137M NaCl; 2.7 mM KCl; 1.5 mM KH₂PO₄; 8 mM Na₂HPO₄; pH 7.2), and subsequently equilibrated with 4 column volumes of buffer C (137 mM NaCl; 2.7 mM KCl; 1.5 mM KH₂PO₄; 8 mM Na₂HPO₄; 20% (w/v) glycerol; pH 7.4). The peak fraction eluted from the Ni-column is automatically loaded onto the Sephadex G-25 column through the integrated sample loader on the VISION and the protein is eluted with buffer C at a flow rate of 2 ml/min. The fraction was filtered through a sterile centrifugation filter (Millipore), frozen and stored at −80° C. An aliquot of the sample is analyzed on SDS-PAGE (4-12% NuPAGE gel; Novex) Western blot with anti-His antibodies. The NuPAGE gel may be stained in a 0.1% Coomassie blue R250 staining solution (30% methanol, 10% acetic acid) at room temperature for 1 h and subsequently destained in 20% methanol, 7.5% acetic acid until the background is clear and the protein bands clearly visible.

Following the electrophoresis the proteins are electrotransferred from the gel to a nitrocellulose membrane. The membrane is blocked with 5% milk powder in buffer E (137 mM NaCl; 2.7 mM KCl; 1.5 mM KH₂PO₄; 8 mM Na₂HPO₄; 0.1% Tween 20, pH 7.4) for 1 h at room temperature, and subsequently incubated with a mixture of 2 rabbit polyclonal anti-His antibodies (G-18 and H-15, 0.2 μg/ml each; Santa Cruz) in 2.5% milk powder in buffer E overnight at 4° C. After a further 1 hour incubation at room temperature, the membrane is washed with buffer E (3×10 min), and then incubated with a secondary HRP-conjugated anti-rabbit antibody (DAKO, HRP 0399) diluted 1/3000 in buffer E containing 2.5% milk powder for 2 hours at room temperature. After washing with buffer E (3×10 minutes), the membrane is developed with the ECL kit (Amersham Pharmacia) for 1 min. The membrane is subsequently exposed to a Hyperfilm (Amersham Pharmacia), the film developed and the western blot image visually analysed.

For samples that show detectable protein bands by Coomassie staining, the protein concentration may be determined using the BCA protein assay kit (Pierce) with bovine serum albumin as standard.

Furthermore, overexpression or knock-down of the expression of the polypeptides in cell lines may be used to determine the effect on transcriptional activation of the host cell genome. Dimerisation partners, co-activators and co-repressors of the INSP106 polypeptide may be identified by immunoprecipitation combined with Western blotting and immunoprecipitation combined with mass spectroscopy.

Sequence Information

SEQ ED: 1 INSP106 exon3nov nucleotide sequence

| | |
|---|---|
| 1 | CCGACTGCAA GTACAAGTTT GAGAACTGGG GTGCGTGTGA |
| | TGGGGGCACA |
| 51 | GGCACCAAAG TCCGCCAAGG CACCCTGAAG AAGGCGCGCT |
| | ACAATGCTCA |
| 101 | GTGCCAGGAG ACCATCCGCG TCACCAAGCC CTGCACCCCC |
| | AAGACCAAAG |
| 151 | CAAAGGCCAA AGGTCAGCGA AAGGAGAAGG GGGTGGGGCT |
| | GTCGCGGGGG |
| 201 | GCTGCCCCCC CCCCCCCCCG CCTGTGA |

SEQ ID: 2 the INSP106 exon 3nov polypeptide

| | |
|---|---|
| 1 | DCKYKFENWG ACDGGTGTKV RQGTLKKARY NAQCQETIRV |
| | TKPCTPKTKA |
| 51 | KAKGQRKEKG VGLSRGAAPP PPRL* |

SEQ ID: 3 INSP106 nucleotide sequence for the INSP106 full length polypeptide including signal peptide

| | |
|---|---|
| 1 | ATGCAGCACC GAGGCTTCCT CCTCCTCACC CTCCTCGCCC |
| | TGCTGGCGCT |
| 51 | CACCTCCGCG GTCGCCAAAA AGAAAGATAA GGTGAAGAAG |
| | GGCGGCCCGG |
| 101 | GGAGCGAGTG CGCTGAGTGG GCCTGGGGGC CCTGCACCCC |
| | CAGCAGCAAG |
| 151 | GATTGCGGCG TGGGTTTCCG CGAGGGCACC TGCGGGGCCC |
| | AGACCCAGCG |
| 201 | CATCCGGTGC AGGGTGCCCT GCAACTGGAA GAAGGAGTTT |
| | GGAGCCGACT |
| 251 | GCAAGTACAA GTTTGAGAAC TGGGGTGCGT GTGATGGGGG |
| | CACAGGCACC |
| 301 | AAAGTCCGCC AAGGCACCCT GAAGAAGGCG CGCTACAATG |
| | CTCAGTGCCA |
| 351 | GGAGACCATC CGCGTCACCA AGCCCTGCAC CCCCAAGACC |
| | AAAGCAAAGG |
| 401 | CCAAAGGTCA GCGAAAGGAG AAGGGGGTGG GGCTGTCGCG |
| | GGGGGCTGCC |
| 451 | CCCCCCCCCC CCCGCCTGTG A |

SEQ ID: 4 the INSP106 full length polypeptide including signal peptide

| | |
|---|---|
| 1 | MQHRGFLLLT LLALLALTSA VAKKKDKVKK GGPGSECAEW |
| | AWGPCTPSSK |
| 51 | DCGVGFREGT CGAQTQRIRC RVPCNWKKEF GADCKYKFEN |
| | WGACDGGTGT |
| 101 | KVRQGTLKKA RYNAQCQETI RVTKPCTPKT KAKAKGQRKE |
| | KGVGLSRGAA |
| 151 | PPPPRL* |

SEQ ID: 5 INSP106 nucleotide sequence for the INSP106 full length polypeptide excluding signal peptide

| | |
|---|---|
| 1 | AAAAAGAAAG ATAAGGTGAA GAAGGGCGGC CCGGGGAGCG |
| | AGTGCGCTGA |
| 51 | GTGGGCCTGG GGGCCCTGCA CCCCCAGCAG CAAGGATTGC |
| | GGCGTGGGTT |
| 101 | TCCGCGAGGG CACCTGCGGG GCCCAGACCC AGCGCATCCG |
| | GTGCAGGGTG |
| 151 | CCCTGCAACT GGAAGAAGGA GTTTGGAGCC GACTGCAAGT |
| | ACAAGTTTGA |
| 201 | GAACTGGGGT GCGTGTGATG GGGGCACAGG CACCAAAGTC |
| | CGCCAAGGCA |
| 251 | CCCTGAAGAA GGCGCGCTAC AATGCTCAGT GCCAGGAGAC |
| | CATCCGCGTC |
| 301 | ACCAAGCCCT GCACCCCCAA GACCAAAGCA AAGGCCAAAG |
| | GTCAGCGAAA |
| 351 | GGAGAAGGGG GTGGGGCTGT CGCGGGGGGC TGCCCCCCCC |
| | CCCCCCCGCC |
| 401 | TGTGA |

SEQ ID: 6 the INSP106 full length polypeptide excluding signal peptide

| | |
|---|---|
| 1 | KKKDKVKKGG PGSECAEWAW GPCTPSSKDC GVGFREGTCG |
| | AQTQRIRCRV |
| 51 | PCNWKKEFGA DCKYKFENWG ACDGGTGTKV RQGTLKKARY |
| | NAQCQETIRV |
| 101 | TKPCTPKTKA KAKGQRKEKG VGLSRGAAPP PPRL* |

SEQ ID: 7 nucleotide sequence for the INSP106 extended portion of exon 3 polypeptide

| | |
|---|---|
| 1 | GTCAGCGAAA GGAGAAGGGG GTGGGGCTGT CGCGGGGGGC |
| | TGCCCCCCCC |
| 51 | CCCCCCCGCC TGTGA |

SEQ ID: 8 the INSP106 extended portion of exon 3 polypeptide

| | |
|---|---|
| 1 | GQRKEKGVGL SRGAAPPPPR L |

SEQ ID: 9 nucleotide sequence of exon 4 of swall|P21741|MK_HUMAN

| | |
|---|---|
| 1 | CCAAGAAAGG GAAGGGAAAG GACTAG |

SEQ ID: 10 amino acid sequence encoded by exon 4 of swall|P21741|MK_HUMAN

| | |
|---|---|
| 1 | AKKGKGKD |

SEQ ID: 11 MDK_known:P21741 nucleotide sequence

| | |
|---|---|
| 1 | ATGCAGCACC GAGGCTTCCT CCTCCTCACC CTCCTCGCCC |
| | TGCTGGCGCT |
| 51 | CACCTCCGCG GTCGCCAAAA AGAAAGATAA GGTGAAGAAG |
| | GGCGGCCCGG |
| 101 | GGAGCGAGTG CGCTGAGTGG GCCTGGGGGC CCTGCACCCC |
| | CAGCAGCAAG |
| 151 | GATTGCGGCG TGGGTTTCCG CGAGGGCACC TGCGGGGCCC |
| | AGACCCAGCG |
| 201 | CATCCGGTGC AGGGTGCCCT GCAACTGGAA GAAGGAGTTT |
| | GGAGCCGACT |
| 251 | GCAAGTACAA GTTTGAGAAC TGGGGTGCGT GTGATGGGGG |
| | CACAGGCACC |
| 301 | AAAGTCCGCC AAGGCACCCT GAAGAAGGCG CGCTACAATG |
| | CTCAGTGCCA |
| 351 | GGAGACCATC CGCGTCACCA AGCCCTGCAC CCCCAAGACC |
| | AAAGCAAAGG |
| 401 | CCAAAGCCAA GAAAGGGAAG GGAAAGGACT AG |

SEQ ID: 12 MDK_known:P21741 amino acid sequence

| | |
|---|---|
| 1 | MQHRGFLLLT LLALLALTSA VAKKKDKVKK GGPGSECAEW |
| | AWGPCTPSSK |
| 51 | DCGVGFREGT CGAQTQRIRC RVPCNWKKEF GADCKYKFEN |
| | WGACDGGTGT |
| 101 | KVRQGTLKKA RYNAQCQETI RVTKPCTPKT KAKAKAKKGK |
| | GKD |

### REFERENCES

1. Akhter, S., Tanaka, I. T., Kojima, S., Muramatsu, H., Inui, T., Kimura, T., Kaneda, N., Talukder, A. H., Muramatsu T (1998) Clusters of Basic Amino Acids in Mididne: Roles in Neurite-Promoting Activity and Plasminogen Activator-Enhancing Activity. J. Biochem. 123, 1127-1136.

2. Iwasaki, W., Nagata, K., Hatanaka, H., Inui, T. Kimura, T., Muramatsu, T., Yoshida, K., Tasumi, M., and Inagaki, F. (1997) Solution structure of midkine, a new heparin binding growth factor. EMBO. 16, 6936-6946.

3. Muramatsu. T. (2002) Midkine and Pleiotrophin: Two related Proteins involved in development, Survival, Inflammation and Tumorigenesis. J. Biochem. 132, 359-371.

4. Pierrot, I. B., Delbe, J., Caruelle, D., Barritault, D., Courty, J., and Milhet P. E. (2001) The Lysine-rich C-terminal Tail of HARP is required fir Mitogenic and Tumor Formation Activites. J. Biol. Chem. 276, 12228-12234.

5. Kojima, S. , Muramatsu, H. , Amanuma, H. and Muramatsu, T. (1995) Midkine enhances fibrinolytic activity of bovine endothelial cells. J. Biol. Chem., 270, 9590-9596

```
# SEQUENCE LIS
#TING
<160> NUMBER OF SEQ ID NOS: 26
<210> SEQ ID NO 1
<211> LENGTH: 227
<212> TYPE: DNA
<213> ORGANISM: Homo Sapiens
<400> SEQUENCE: 1
ccgactgcaa gtacaagttt gagaactggg gtgcgtgtga tgggggcaca gg
#caccaaag 60
tccgccaagg caccctgaag aaggcgcgct acaatgctca gtgccaggag ac
#catccgcg 120
tcaccaagcc ctgcaccccc aagaccaaag caaaggccaa aggtcagcga aa
#ggagaagg 180
gggtggggct gtcgcggggg gctgcccccc cccccccccg cctgtga
# 227
<210> SEQ ID NO 2
<211> LENGTH: 74
<212> TYPE: PRT
<213> ORGANISM: Homo sapiens
<400> SEQUENCE: 2
Asp Cys Lys Tyr Lys Phe Glu Asn Trp Gly Al
#a Cys Asp Gly Gly Thr
1 5
# 10
# 15
Gly Thr Lys Val Arg Gln Gly Thr Leu Lys Ly
#s Ala Arg Tyr Asn Ala
                        20
# 25
# 30
Gln Cys Gln Glu Thr Ile Arg Val Thr Lys Pr
#o Cys Thr Pro Lys Thr
                35
# 40
# 45
Lys Ala Lys Ala Lys Gly Gln Arg Lys Glu Ly
#s Gly Val Gly Leu Ser
        50
# 55
# 60
Arg Gly Ala Ala Pro Pro Pro Pro Arg Leu
65
#70
<210> SEQ ID NO 3
<211> LENGTH: 471
<212> TYPE: DNA
<213> ORGANISM: Homo sapiens
<400> SEQUENCE: 3
atgcagcacc gaggcttcct cctcctcacc ctcctcgccc tgctggcgct ca
#cctccgcg 60
gtcgccaaaa agaaagataa ggtgaagaag ggcggcccgg ggagcgagtg cg
#ctgagtgg 120
gcctgggggc cctgcacccc cagcagcaag gattgcggcg tgggtttccg cg
#agggcacc 180
tgcggggccc agacccagcg catccggtgc agggtgccct gcaactggaa ga
#aggagttt 240
ggagccgact gcaagtacaa gtttgagaac tggggtgcgt gtgatggggg ca
#caggcacc 300
aaagtccgcc aaggcaccct gaagaaggcg cgctacaatg ctcagtgcca gg
#agaccatc 360
cgcgtcacca agccctgcac ccccaagacc aaagcaaagg ccaaaggtca gc
#gaaaggag 420
aagggggtgg ggctgtcgcg gggggctgcc cccccccccc cccgcctgtg a
# 471
<210> SEQ ID NO 4
<211> LENGTH: 156
<212> TYPE: PRT
<213> ORGANISM: Homo sapiens
<400> SEQUENCE: 4
Met Gln His Arg Gly Phe Leu Leu Leu Thr Le
#u Leu Ala Leu Leu Ala
1 5
# 10
# 15
Leu Thr Ser Ala Val Ala Lys Lys Lys Asp Ly
#s Val Lys Lys Gly Gly
                        20
# 25
# 30
Pro Gly Ser Glu Cys Ala Glu Trp Ala Trp Gl
#y Pro Cys Thr Pro Ser
                35
# 40
# 45
Ser Lys Asp Cys Gly Val Gly Phe Arg Glu Gl
#y Thr Cys Gly Ala Gln
        50
# 55
# 60
Thr Gln Arg Ile Arg Cys Arg Val Pro Cys As
#n Trp Lys Lys Glu Phe
65
#70
#75
#80
Gly Ala Asp Cys Lys Tyr Lys Phe Glu Asn Tr
#p Gly Ala Cys Asp Gly
                                85
# 90
# 95
Gly Thr Gly Thr Lys Val Arg Gln Gly Thr Le
#u Lys Lys Ala Arg Tyr
                        100
# 105
# 110
Asn Ala Gln Cys Gln Glu Thr Ile Arg Val Th
#r Lys Pro Cys Thr Pro
                115
# 120
# 125
Lys Thr Lys Ala Lys Ala Lys Gly Gln Arg Ly
#s Glu Lys Gly Val Gly
        130
# 135
# 140
Leu Ser Arg Gly Ala Ala Pro Pro Pro Pro Ar
#g Leu
145 1
#50 1
#55
<210> SEQ ID NO 5
<211> LENGTH: 405
<212> TYPE: DNA
<213> ORGANISM: Homo sapiens
<400> SEQUENCE: 5
aaaaagaaag ataaggtgaa gaagggcggc ccggggagcg agtgcgctga gt
#gggcctgg 60
gggccctgca cccccagcag caaggattgc ggcgtgggtt tccgcgaggg ca
#cctgcggg 120
gcccagaccc agcgcatccg gtgcagggtg ccctgcaact ggaagaagga gt
#ttggagcc 180
gactgcaagt acaagtttga gaactggggt gcgtgtgatg ggggcacagg ca
#ccaaagtc 240
cgccaaggca ccctgaagaa ggcgcgctac aatgctcagt gccaggagac ca
#tccgcgtc 300
accaagccct gcacccccaa gaccaaagca aaggccaaag gtcagcgaaa gg
#agaagggg 360
gtggggctgt cgcggggggc tgcccccccc cccccccgcc tgtga
# 405
<210> SEQ ID NO 6
<211> LENGTH: 134
<212> TYPE: PRT
<213> ORGANISM: Homo sapiens
<400> SEQUENCE: 6
Lys Lys Lys Asp Lys Val Lys Lys Gly Gly Pr
#o Gly Ser Glu Cys Ala
1 5
# 10
# 15
Glu Trp Ala Trp Gly Pro Cys Thr Pro Ser Se
#r Lys Asp Cys Gly Val
                        20
# 25
# 30
Gly Phe Arg Glu Gly Thr Cys Gly Ala Gln Th
#r Gln Arg Ile Arg Cys
                35
# 40
# 45
Arg Val Pro Cys Asn Trp Lys Lys Glu Phe Gl
#y Ala Asp Cys Lys Tyr
        50
# 55
# 60
Lys Phe Glu Asn Trp Gly Ala Cys Asp Gly Gl
#y Thr Gly Thr Lys Val
65
#70
#75
#80
Arg Gln Gly Thr Leu Lys Lys Ala Arg Tyr As
#n Ala Gln Cys Gln Glu
                                85
# 90
# 95
Thr Ile Arg Val Thr Lys Pro Cys Thr Pro Ly
#s Thr Lys Ala Lys Ala
                        100
# 105
# 110
Lys Gly Gln Arg Lys Glu Lys Gly Val Gly Le
#u Ser Arg Gly Ala Ala
                115
# 120
# 125
Pro Pro Pro Pro Arg Leu
        130
<210> SEQ ID NO 7
<211> LENGTH: 65
<212> TYPE: DNA
<213> ORGANISM: Homo sapiens
<400> SEQUENCE: 7
gtcagcgaaa ggagaagggg gtggggctgt cgcggggggc tgcccccccc cc
#cccccgcc 60
tgtga
#
#
# 65
<210> SEQ ID NO 8
<211> LENGTH: 21
<212> TYPE: PRT
<213> ORGANISM: Homo sapiens
<400> SEQUENCE: 8
Gly Gln Arg Lys Glu Lys Gly Val Gly Leu Se
#r Arg Gly Ala Ala Pro
1 5
# 10
# 15
Pro Pro Pro Arg Leu
                        20
<210> SEQ ID NO 9
<211> LENGTH: 26
<212> TYPE: DNA
<213> ORGANISM: Homo sapiens
<400> SEQUENCE: 9
ccaagaaagg gaagggaaag gactag
#
# 26
<210> SEQ ID NO 10
<211> LENGTH: 8
<212> TYPE: PRT
<213> ORGANISM: Homo sapiens
<400> SEQUENCE: 10
Ala Lys Lys Gly Lys Gly Lys Asp
1 5
<210> SEQ ID NO 11
<211> LENGTH: 432
<212> TYPE: DNA
<213> ORGANISM: Homo sapiens
<400> SEQUENCE: 11
atgcagcacc gaggcttcct cctcctcacc ctcctcgccc tgctggcgct ca
#cctccgcg 60
gtcgccaaaa agaaagataa ggtgaagaag ggcggcccgg ggagcgagtg cg
#ctgagtgg 120
gcctgggggc cctgcacccc cagcagcaag gattgcggcg tgggtttccg cg
#agggcacc 180
tgcggggccc agacccagcg catccggtgc agggtgccct gcaactggaa ga
#aggagttt 240
ggagccgact gcaagtacaa gtttgagaac tggggtgcgt gtgatggggg ca
#caggcacc 300
aaagtccgcc aaggcaccct gaagaaggcg cgctacaatg ctcagtgcca gg
#agaccatc 360
cgcgtcacca agccctgcac ccccaagacc aaagcaaagg ccaaagccaa ga
#aagggaag 420
ggaaaggact ag
#
#
# 432
<210> SEQ ID NO 12
<211> LENGTH: 143
<212> TYPE: PRT
<213> ORGANISM: Homo sapiens
<400> SEQUENCE: 12
Met Gln His Arg Gly Phe Leu Leu Leu Thr Le
#u Leu Ala Leu Leu Ala
1 5
# 10
# 15
Leu Thr Ser Ala Val Ala Lys Lys Lys Asp Ly
#s Val Lys Lys Gly Gly
                        20
# 25
# 30
Pro Gly Ser Glu Cys Ala Glu Trp Ala Trp Gl
#y Pro Cys Thr Pro Ser
                35
# 40
# 45
Ser Lys Asp Cys Gly Val Gly Phe Arg Glu Gl
#y Thr Cys Gly Ala Gln
        50
# 55
# 60
Thr Gln Arg Ile Arg Cys Arg Val Pro Cys As
#n Trp Lys Lys Glu Phe
65
#70
#75
#80
Gly Ala Asp Cys Lys Tyr Lys Phe Glu Asn Tr
#p Gly Ala Cys Asp Gly
                                85
# 90
# 95
Gly Thr Gly Thr Lys Val Arg Gln Gly Thr Le
#u Lys Lys Ala Arg Tyr
                        100
# 105
# 110
Asn Ala Gln Cys Gln Glu Thr Ile Arg Val Th
#r Lys Pro Cys Thr Pro
                115
# 120
# 125
Lys Thr Lys Ala Lys Ala Lys Ala Lys Lys Gl
#y Lys Gly Lys Asp
        130
# 135
# 140
<210> SEQ ID NO 13
<211> LENGTH: 22
<212> TYPE: DNA
<213> ORGANISM: Artificial Sequence
<220> FEATURE:
<223> OTHER INFORMATION: INSP106-CP1 Primer
<400> SEQUENCE: 13
caggatgcag caccgaggct tc
#
# 22
<210> SEQ ID NO 14
<211> LENGTH: 22
<212> TYPE: DNA
<213> ORGANISM: Artificial Sequence
<220> FEATURE:
<223> OTHER INFORMATION: INSP106-CP2 Primer
<400> SEQUENCE: 14
gccaagtgag gcgatgtcag ga
#
# 22
<210> SEQ ID NO 15
<211> LENGTH: 19
<212> TYPE: DNA
<213> ORGANISM: Artificial Sequence
<220> FEATURE:
<223> OTHER INFORMATION: INSP106-SP1 Primer
<400> SEQUENCE: 15
cctgcaactg gaagaagga
#
#
# 19
<210> SEQ ID NO 16
<211> LENGTH: 20
<212> TYPE: DNA
<213> ORGANISM: Artificial Sequence
<220> FEATURE:
<223> OTHER INFORMATION: INSP106-SP2 Primer
<400> SEQUENCE: 16
ttggcggact ttggtgcctg
#
#
# 20
<210> SEQ ID NO 17
<211> LENGTH: 35
<212> TYPE: DNA
<213> ORGANISM: Artificial Sequence
<220> FEATURE:
<223> OTHER INFORMATION: INSP106-EX1 Primer
<400> SEQUENCE: 17
gcaggcttcg ccaccatgca gcaccgaggc ttcct
#
# 35
<210> SEQ ID NO 18
<211> LENGTH: 35
<212> TYPE: DNA
<213> ORGANISM: Artificial Sequence
<220> FEATURE:
<223> OTHER INFORMATION: INSP106-EX2 Primer
<400> SEQUENCE: 18
gtgatggtga tggtgcaggc gtggaggtgg ggggg
#
# 35
<210> SEQ ID NO 19
<211> LENGTH: 37
<212> TYPE: DNA
<213> ORGANISM: Artificial Sequence
<220> FEATURE:
<223> OTHER INFORMATION: GCP Forward Primer
<400> SEQUENCE: 19
ggggacaagt ttgtacaaaa aagcaggctt cgccacc
#
# 37
<210> SEQ ID NO 20
<211> LENGTH: 51
<212> TYPE: DNA
<213> ORGANISM: Artificial Sequence
<220> FEATURE:
<223> OTHER INFORMATION: GCP Reverse Primer
<400> SEQUENCE: 20
ggggaccact ttgtacaaga aagctgggtt tcaatggtga tggtgatggt g
# 51
<210> SEQ ID NO 21
<211> LENGTH: 20
<212> TYPE: DNA
<213> ORGANISM: Artificial Sequence
<220> FEATURE:
<223> OTHER INFORMATION: pEAK12-F Primer
<400> SEQUENCE: 21
gccagcttgg cacttgatgt
#
#
# 20
<210> SEQ ID NO 22
<211> LENGTH: 20
<212> TYPE: DNA
<213> ORGANISM: Artificial Sequence
<220> FEATURE:
<223> OTHER INFORMATION: pEAK12-R Primer
<400> SEQUENCE: 22
gatggaggtg gacgtgtcag
#
#
# 20
<210> SEQ ID NO 23
<211> LENGTH: 25
<212> TYPE: DNA
<213> ORGANISM: Artificial Sequence
<220> FEATURE:
<223> OTHER INFORMATION: pENTR-F Primer
<400> SEQUENCE: 23
tcgcgttaac gctagcatgg atctc
#
# 25
<210> SEQ ID NO 24
<211> LENGTH: 24
<212> TYPE: DNA
<213> ORGANISM: Artificial Sequence
<220> FEATURE:
<223> OTHER INFORMATION: pENTR-R Primer
<400> SEQUENCE: 24
gtaacatcag agattttgag acac
#
# 24
<210> SEQ ID NO 25
<211> LENGTH: 20
<212> TYPE: DNA
<213> ORGANISM: Artificial Sequence
<220> FEATURE:
<223> OTHER INFORMATION: T7 Primer
<400> SEQUENCE: 25
taatacgact cactataggg
#
#
# 20
<210> SEQ ID NO 26
<211> LENGTH: 21
<212> TYPE: DNA
<213> ORGANISM: Artificial Sequence
<220> FEATURE:
<223> OTHER INFORMATION: T3 Primer
<400> SEQUENCE: 26
ctccctttag tgagggtaat t
#
#
#21
```

We claim:

1-45. (canceled)

46. A composition of matter comprising:
a) an isolated polypeptide that does not comprise the amino acid sequence recited in SEQ ID NO:10, wherein said isolated polypeptide is selected from the group consisting of:
1) an amino acid sequence comprising that recited in SEQ ID NO:2;
2) a fragment of SEQ ID NO:2, wherein said fragment comprises at least a fragment of SEQ ID NO:8, and wherein said isolated polypeptide has the activity of SEQ ID NO:4 or SEQ ID NO:6 or has an antigenic determinant that is specific to a polypeptide comprising SEQ ID NO:2;
3) a functional equivalent of 1) or 2), wherein the functional equivalent has the activity of SEQ ID NO:4 or SEQ ID NO:6 or has an antigenic determinant that is specific to a polypeptide comprising SEQ ID NO:2;
4) an amino acid sequence comprising that recited in SEQ ID NO:4 or SEQ ID NO:6;
5) an amino acid sequence consisting of that recited in SEQ ID NO:4 or SEQ ID NO:6;
6) a fragment of SEQ ID NO:4 or SEQ ID NO:6, wherein the fragment comprises SEQ ID NO:8 or SEQ ID NO:2, and wherein the isolated polypeptide has the activity of SEQ ID NO:4 or SEQ ID NO:6 or has an antigenic determinant that is specific to a polypeptide comprising SEQ ID NO:2;
7) a functional equivalent of 4), 5), or 6), wherein the functional equivalent has the activity of SEQ ID NO:4 or SEQ ID NO:6 or has an antigenic determinant that is specific to a polypeptide comprising SEQ ID NO:2, and wherein the functional equivalent comprises SEQ ID NO:8 or SEQ ID NO:2;
8) the functional equivalent of 3) or 7), characterized in that it has at least 70% sequence identity to the amino acid sequence recited in SEQ ID NO:2, SEQ ID NO:4, SEQ ID NO:6, or SEQ ID NO:8;
9) the functional equivalent of 3) or 7), characterized in that it has at least 90% sequence identity to the amino acid sequence recited in SEQ ID NO:2, SEQ ID NO:4, SEQ ID NO:6, or SEQ ID NO:8;
10) the fragment or functional equivalent of 2), 3), 6), or 7), which further comprises a sequence having a degree of sequence identity with the amino acid sequence recited in SEQ ID NO:8 of greater than 60%;
11) the fragment or functional equivalent of 2), 3), 6), or 7), wherein the fragment or functional equivalent comprises a sequence having a degree of sequence identity with the amino acid sequence recited in SEQ ID NO:8 of greater than 90%;
12) the functional equivalent of 3), 7), 10), or 11), wherein the functional equivalent comprises the amino acid sequence recited in SEQ ID NO:8; and
13) the fragment of 2), 6), 10), or 11), wherein the fragment has an antigenic determinant that is specific to a polypeptide comprising SEQ ID NO:2 which consists of 7 or more amino acid residues from the amino acid sequence recited in SEQ ID NO:2 or SEQ ID NO:8; or
b) a purified mRNA or cDNA nucleic acid molecule, or a nucleic acid molecule complementary thereto:
1) encoding, or whose complement encodes, a polypeptide of any of a1) to a13); or
2) comprising the nucleic acid sequence recited in SEQ ID NO: 1, SEQ ID NO:3, SEQ ID NO:5, or SEQ ID NO:7, or is a redundant equivalent or fragment of any of the foregoing; or
3) consisting of the nucleic acid sequence recited in SEQ ID NO:1, SEQ ID NO:3, SEQ ID NO:5, or SEQ ID NO:7, or is a redundant equivalent or fragment of any of the foregoing; or
4) that hybridizes under high stringency conditions with SEQ ID NO:3 or SEQ ID NO:5, but which does not hybridize under high stringency conditions to SEQ ID NO: 11; or
c) a vector comprising a nucleic acid molecule according to any one of b1) to b4); or
d) a host cell transformed with a vector or a nucleic acid molecule according to any one of b) or c); or
e) a ligand:
1) that binds specifically to the polypeptide of any of a1) to a13); or
2) which is an antibody that binds specifically to the polypeptide of any of a1) to a13); or
f) a compound:
1) that increases the level of expression or activity of a polypeptide according to any of a1) to a13); or
2) that decreases the level of expression or activity of a polypeptide according to any of a1) to a13); or
g) a compound that binds to a polypeptide according to any of a1) to a13) without inducing any of the biological effects of the polypeptide; or
h) a compound that binds to a polypeptide according to any of a1) to a13) without inducing any of the biological effects of the polypeptide, wherein the compound is a natural or modified substrate, ligand, enzyme, receptor or structural or functional mimetic; or
i) a pharmaceutical composition comprising any one of a) to h), and a pharmaceutically acceptable carrier; or
j) a vaccine composition comprising any one of a1) to a13) or b1) to b4); or
k) a kit for diagnosing disease, comprising a first container containing a nucleic acid probe that hybridizes under stringent conditions with a nucleic acid molecule of any one of b1) to b4), a second container containing primers useful for amplifying the nucleic acid molecule, and instructions for using the probe and primers for facilitating the diagnosis of disease; or
l) a kit for diagnosing disease, comprising a first container containing a nucleic acid probe that hybridizes under stringent conditions with a nucleic acid molecule of any one of b1) to b4); a second container containing primers useful for amplifying the nucleic acid molecule; a third container holding an agent for digesting unhybridized RNA; and instructions for using the probe and primers for facilitating the diagnosis of disease; or
m) a kit comprising an array of nucleic acid molecules, at least one of which is a nucleic acid molecule according to any one of b1) to b4); or
n) a kit comprising one or more antibodies that bind to a polypeptide as recited in any one of a1) to a13); and areagent useful for the detection of a binding reaction between the one or more antibodies and the polypeptide; or
o) a transgenic or knockout non-human animal that has been transformed to express higher, lower, or absent levels of a polypeptide according to any one of a1) to a13).

47. A method of using a composition of matter, comprising obtaining a composition of matter according to claim 46 and using said composition of matter in a method selected from: diagnosing a disease in a patient; treatment of a disease in a patient; monitoring the therapeutic treatment of a disease; identification of a compound that is effective in the treatment and/or diagnosis of a disease; and screening candidate compounds.

48. The method of claim 47, wherein said method of using a composition of matter comprises the method for treatment of a disease, comprising administering to the patient:
a) an isolated polypeptide that does not comprise the amino acid sequence recited in SEQ ID NO: 10, wherein said isolated polypeptide is selected from the group consisting of:
1) an amino acid sequence comprising that recited in SEQ ID NO:2;
2) a fragment of SEQ ID NO:2, wherein said fragment comprises at least a fragment of SEQ ID NO:8, and wherein said isolated polypeptide has the activity of SEQ ID NO:4 or SEQ ID NO:6 or has an antigenic determinant that is specific to a polypeptide comprising SEQ ID NO:2;
3) a functional equivalent of 1) or 2), wherein the functional equivalent has the activity of SEQ ID NO:4 or SEQ ID NO:6 or has an antigenic determinant that is specific to a polypeptide comprising SEQ ID NO:2;
4) an amino acid sequence comprising that recited in SEQ ID NO:4 or SEQ ID NO:6;
5) an amino acid sequence consisting of that recited in SEQ ID NO:4 or SEQ ID NO:6;
6) a fragment of SEQ ID NO:4 or SEQ ID NO:6, wherein the fragment comprises SEQ ID NO:8 or SEQ ID NO:2, and wherein the isolated polypeptide has the activity of SEQ ID NO:4 or SEQ ID NO:6 or has an antigenic determinant that is specific to a polypeptide comprising SEQ ID NO:2;
7) a functional equivalent of 4), 5), or 6), wherein the functional equivalent has the activity of SEQ ID NO:4 or SEQ ID NO:6 or has an antigenic determinant that is specific to a polypeptide comprising SEQ ID NO:2, and wherein the functional equivalent comprises SEQ ID NO:8 or SEQ ID NO:2;
8) the functional equivalent of 3) or 7), characterized in that it has at least 70% sequence identity to the amino acid sequence recited in SEQ ID NO:2, SEQ ID NO:4, SEQ ID NO:6, or SEQ ID NO:8;
9) the functional equivalent of 3) or 7), characterized in that it has at least 90% sequence identity to the amino acid sequence recited in SEQ ID NO:2, SEQ ID NO:4, SEQ ID NO:6, or SEQ ID NO:8;
10) the fragment or functional equivalent of 2), 3), 6), 7), which further comprises a sequence having a degree of sequence identity with the amino acid sequence recited in SEQ ID NO:8 of greater than 60%;
11) the fragment or functional equivalent of 2), 3), 6), 7), wherein the fragment or functional equivalent comprises a sequence having a degree of sequence identity with the amino acid sequence recited in SEQ ID NO:8 of greater than 90%;
12) the functional equivalent of 3), 7), 10), or 11), wherein the functional equivalent comprises the amino acid sequence recited in SEQ ID NO:8; and
13) the fragment of 2), 6), 10), or 11), wherein the fragment has an antigenic determninant that is specific to a polypeptide comprising SEQ ID NO:2 which consists of 7 or more amino acid residues from the amino acid sequence recited in SEQ ID NO:2 or SEQ ID NO:8; or
b) a purified mRNA or cDNA nucleic acid molecule, or a nucleic acid molecule complementary thereto:
1) encoding, or whose complement encodes, a polypeptide of any of a1) to a13); or
2) comprising the nucleic acid sequence recited in SEQ ID NO: 1, SEQ ID NO:3, SEQ ID NO:5, or SEQ ID NO:7, or is a redundant equivalent or fragment of any of the foregoing; or
3) consisting of the nucleic acid sequence recited in SEQ ID NO: 1, SEQ ID NO:3, SEQ ID NO:5, or SEQ ID NO:7, or is a redundant equivalent or fragment of any of the foregoing; or
4) that hybridizes under high stringency conditions with SEQ ID NO:3 or SEQ ID NO:5, but which does not hybridize under high stringency conditions to SEQ ID NO: 11; or
c) a vector comprising a nucleic acid molecule according to any one of b1) to b4); or
d) a host cell transformed with a vector or a nucleic acid molecule according to any one of b) or c); or
e) a ligand:
1) that binds specifically to the polypeptide of any of a1) to a13); or
2) which is an antibody that binds specifically to the polypeptide of any of a1) to a13); or
f) a compound:
1) that increases the level of expression or activity of a polypeptide according to any of a1) to a13); or
2) that decreases the level of expression or activity of a polypeptide according to any of a1) to a13); or
g) a compound that binds to a polypeptide according to any of a1) to a13) without inducing any of the biological effects of the polypeptide; or
h) a compound that binds to a polypeptide according to any of a1) to a13) without inducing any of the biological effects of the polypeptide, wherein the compound is a natural or modified substrate, ligand, enzyme, receptor or structural or functional mimetic; or
i) a pharmaceutical composition comprising any one of a) to h), and a pharmaceutically acceptable carrier.

49. The method of claim 48, wherein said method of using a composition of matter comprises the method for treatment of a disease, and wherein the disease includes reproductive disorders, cell proliferative disorders, including neoplasm, melanoma, lung, colorectal, breast, pancreas, head and neck and other solid tumours; stomach cancer, colon cancer, pancreatic cancer, lung cancer, thoracic cancer, and liver cancer; myeloproliferative disorders, such as leukemia, non-Hodgkin lymphoma, leukopenia, thrombocytopenia, angiogenesis disorder, Kaposis' sarcoma; autoimmune/inflammatory disorders, including allergy, inflammatory bowel disease, pancreatitis, arthritis, psoriasis, psoriasis vulgaris, respiratory tract inflammation, asthma, and organ transplant rejection; cardiovascular disorders, including hypertension, oedema, angina, atherosclerosis, thrombosis, sepsis, shock, reperfusion injury, and ischemia, particularly ischemic heart disease; neurological disorders including central nervous system disease, Alzheimer's disease, brain injury, Parkinson's disease, amyotrophic lateral sclerosis, and pain; developmental disorders; metabolic disorders including diabetes mellitus, osteoporosis, and obesity, AIDS, renal disease, particularly idiopathic nephrotic syndrome; disorders related to fibrinolysis; neutrophilic functional disorders (such as lazy-leukocyte (chemotaxis-deficient leukocyte) syndrome); inflammatory diseases; wound healing disorders; lung injury; infections including viral infection, bacterial infection, fungal infection and parasitic infection and other pathological conditions.

50. The method of claim 48, wherein said method of using a composition of matter comprises the method for treatment of a disease, and wherein the disease is one for which the expression of the natural gene or the activity of the polypeptide is lower in a diseased patient when compared to the level of expression or activity in a healthy patient, the polypeptide, nucleic acid molecule, vector, ligand, compound or composition administered to the patient is an agonist.

51. The method of claim 48, wherein said method of using a composition of matter comprises the method for treatment of a disease, and wherein the disease is one for which expression of the natural gene or activity of the polypeptide is higher in a diseased patient when compared to the level of expression or activity in a healthy patient, the polypeptide, nucleic acid molecule, vector, ligand, compound or composition administered to the patient is an antagonist.

52. The method of claim 47, wherein said method of using a composition of matter comprises the method for diagnosing a disease in a patient, comprising assessing the level of expression of a natural gene encoding a polypeptide of claim 46, or assessing the activity of a polypeptide of claim 46, in tissue from said patient; and comparing said level of expression or activity to a control level, wherein a level that is different to said control level is indicative of disease.

53. The method of claim 52, which is carried out in vitro.

54. The method of claim 52, comprising the steps of:
a) contacting a ligand of claim 46 with a biological sample under conditions suitable for the formation of a ligand-polypeptide complex; and
b) detecting said complex.

55. The method of claim 52, comprising the steps of:
a) contacting a sample oftissue from the patient with a nucleic acid probe under stringent conditions that allow the formation of a hybrid complex between a nucleic acid molecule of claim 46 and the probe;
b) contacting a control sample with said probe under the same conditions used in step a); and
c) detecting the presence of hybrid complexes in said samples; wherein detection of levels of the hybrid complex in the patient sample that differ from levels of the hybrid complex in the control sample is indicative of disease.

56. The method of claim 52, comprising the steps of:
a) contacting a sample of nucleic acid from tissue of the patient with a nucleic acid primer under stringent conditions that allow the formation of a hybrid complex between a nucleic acid molecule of claim 46 and the primer;
b) contacting a control sample with said primer under the same conditions used in step a); and
c) amplifying the sampled nucleic acid; and
d) detecting the level of amplified nucleic acid from both patient and control samples; wherein detection of levels of the amplified nucleic acid in the patient sample that differ significantly from levels of the amplified nucleic acid in the control sample is indicative of disease.

57. The method of claim 52, comprising:
a) obtaining a tissue sample from a patient being tested for disease;
b) isolating a nucleic acid molecule of claim 46 from said tissue sample; and
c) diagnosing the patient for disease by detecting the presence of a mutation which is associated with disease in the nucleic acid molecule as an indication of the disease.

58. The method of claim 57, further comprising amplifying the nucleic acid molecule to form an amplified product and detecting the presence or absence of a mutation in the amplified product.

59. The method of claim 57, wherein the presence or absence of the mutation in the patient is detected by contacting said nucleic acid molecule with a nucleic acid probe that hybridizes to said nucleic acid molecule under stringent conditions to form a hybrid double-stranded molecule, the hybrid double-stranded molecule having an unhybridized portion of the nucleic acid probe strand at any portion corresponding to a mutation associated with disease; and detecting the presence or absence of an unhybridized portion of the probe strand as an indication of the presence or absence of a disease-associated mutation.

60. The method of claim 52, wherein said disease includes reproductive disorders, cell proliferative disorders, including neoplasm, melanoma, lung, colorectal, breast, pancreas, head and neck and other solid tumours; stomach cancer, colon cancer, pancreatic cancer, lung cancer, thoracic cancer, and liver cancer; myeloproliferative disorders, such as leukemia, non-Hodgkin lymphoma, leukopenia, thrombocytopenia, angiogenesis disorder, Kaposis' sarcoma; autoimmune/inflammatory disorders, including allergy, inflammatory bowel disease, pancreatitis, arthritis, psoriasis, psoriasis vulgaris, respiratory tract inflammation, asthma, and organ transplant rejection; cardiovascular disorders, including hypertension, oedema, angina, atherosclerosis, thrombosis, sepsis, shock, reperfusion injury, and ischemia, particularly ischemic heart disease; neurological disorders including central nervous system disease, Alzheimer's disease, brain injury, Parkinson's disease, amyotrophic lateral sclerosis, and pain; developmental disorders; metabolic disorders including diabetes mellitus, osteoporosis, and obesity, AIDS, renal disease, particularly idiopathic nephrotic syndrome; disorders related to fibrinolysis; neutrophilic functional disorders (such as lazy-leukocyte (chemotaxis-deficient leukocyte) syndrome); inflammatory diseases; wound healing disorders; lung injury; infections including viral infection, bacterial infection, fungal infection and parasitic infection and other pathological conditions.

61. The method of claim 52, wherein said disease is a disease in which midkines are implicated.

62. The method of claim 47, wherein said method of using a composition of matter comprises the method of monitoring the therapeutic treatment of a disease, comprising monitoring over a period of time the level of expression or activity of a polypeptide of claim 46, or the level of expression of a nucleic acid molecule of claim 46 in tissue from said patient, wherein altering said level of expression or activity over the period of time towards a control level is indicative of regression of said disease.

63. The method of claim 47, wherein said method of using a composition of matter comprises the method for identification of a compound that is effective in the treatment and/or diagnosis of a disease, comprising contacting a polypeptide of claim 46 or a nucleic acid molecule of claim 46 with one or more compounds suspected of possessing binding affinity for said polypeptide or nucleic acid molecule, and selecting a compound that binds specifically to said nucleic acid molecule or polypeptide.

64. The method of claim 47, wherein said method of using a composition of matter comprises the method for screening candidate compounds, comprising contacting a non-human transgenic animal of claim 46 with a candidate compound and determining the effect of the compound on the disease of the animal.

65. An isolated polypeptide comprising the amino acid sequence recited in SEQ ID NO:2, wherein said polypeptide does not comprise the amino acid sequence recited in SEQ ID NO: 10.

66. An isolated polypeptide consisting of the amino acid sequence recited in SEQ ID NO:4 or SEQ ID NO:6, wherein said polypeptide does not comprise the amino acid sequence recited in SEQ ID NO: 10.
